(19) United States
(12) Patent Application Publication
(10) Pub. No.: US 2025/0049512 A1
(43) Pub. Date: Feb. 13, 2025

(54) Dynamic Visualization For Device Delivery

(71) Applicant: LightLab Imaging, Inc., Westford, MA (US)

(72) Inventors: Humphrey Chen, Acton, MA (US); Ajay Gopinath, Bedford, MA (US); Gregory Patrick Amis, Westford, MA (US); Avi Motova, Haifa (IL); Songyuan Tang, Westford, MA (US); Chiedza Chauruka, Burlington, MA (US); Chih-Hao Liu, Chelmsford, MA (US); Karl W. Engel, Redwood City, CA (US)

(73) Assignee: LightLab Imaging, Inc., Westford, MA (US)

(21) Appl. No.: 18/798,322

(22) Filed: Aug. 8, 2024

Related U.S. Application Data
(63) Continuation of application No. PCT/US2024/039350, filed on Jul. 24, 2024.
(60) Provisional application No. 63/659,536, filed on Jun. 13, 2024.
(60) Provisional application No. 63/603,931, filed on Nov. 29, 2023.
(60) Provisional application No. 63/531,345, filed on Aug. 8, 2023.

(51) Int. Cl. A61B 34/20; G06T 7/00; G06T 7/246; G06T 7/73; G16H 30/40

(52) U.S. Cl. CPC A61B 34/20; G06T 7/0012; G06T 7/248; G06T 7/74; G16H 30/40; A61B 2034/2065; G06T 2207/10121; G06T 2207/20032; G06T 2207/20104; G06T 2207/30048; G06T 2207/30096; G06T 2207/30101; G06T 2207/30204; G06T 2207/30241

(57) ABSTRACT

The present disclosure provides systems and methods for dynamically visualizing the delivery of a device within a vessel by correlating at least one first extraluminal image with second extraluminal images. The extraluminal images may be correlated based on motion features, without the use of other sensors or timestamps. The first extraluminal image may be a high dose contrast x-ray angiogram (“XA”) and the second extraluminal images may be low dose contrast XAs. The high dose contrast XA may be used to generate a vessel map. The low dose contrast XAs may be taken during the delivery of a device, such as a balloon, stent, probe, or the like. Correlating the high dose XA and low dose XA based on motion features allows for the vessel map to be overlaid on the low dose XA to provide the physician visualization of where the device is within the vessel tree in real time.

### CROSS-REFERENCE TO RELATED APPLICATIONS

The present application is a continuation of International Application No. PCT/US2024/039350, filed Jul. 24, 2024, which claims the benefit of the filing date of U.S. Provisional Patent Application No. 63/531,345, filed Aug. 8, 2023, U.S. Provisional Patent Application No. 63/603,931, filed Nov. 29, 2023, and U.S. Provisional Patent Application No. 63/659,536, filed Jun. 13, 2024, the disclosures of which are incorporated herein by reference.

### BACKGROUND

Blood vessels are typically difficult to see on an x-ray angiogram without the use of a radiocontrast agent (which may be referred to more simply as “contrast agent” or just as “contrast”) within the vessels, such as an iodinated contrast. When delivering an intravascular device, such as a balloon, stent or probe, within a blood vessel, physicians typically refer to a vessel tree generated from an x-ray angiogram (XA) captured with a high level of radiocontrast agent present to better define the vessel tree in the XA. However, the intravascular device itself is typically not visible in an x-ray angiogram captured using such a high level of contrast agent, and repeated or continuous use of contrast agent during a procedure is undesirable at least for medical reasons. Typically, when delivering an intravascular device, x-ray angiograms may be captured with low levels of, or no, contrast agent present in the blood vessels of interest, so that the position of the device can be readily seen using the x-ray angiograms. Then from time to time, to better see where the device is located within the vessel tree, the physician may inject more contrast agent into the blood vessel(s) of interest. The heart function typically clears the contrast agent after less than a second. To provide for continued observation of the vessel tree during the delivery of the device, the physician can either repeatedly inject more contrast agent, which can lead to unwanted side-effects, or be left unable to see the vessel tree when delivering the device.

### BRIEF SUMMARY

The disclosure is generally directed to dynamically visualizing the delivery of a device within a blood vessel, for example within the context of a blood vessel tree or other region of interest, by correlating at least one first extraluminal image with second extraluminal images. The extraluminal images may be correlated based on motion features, visible within or derivable from the extraluminal images without the use of other sensors, such as readings from an electrocardiogram, or time stamps. The at least one first extraluminal image may be a high dose contrast x-ray angiogram (“XA”) captured using a high level of contrast agent present so as to make the blood vessel or blood vessel tree clearly visible in this XA (referred to herein as a high dose contrast XA or a high dose XA). The second extraluminal images may then be low dose XAs taken using little or no contrast agent present in the blood vessel or blood vessel tree so that the intravascular device is more clearly visible (referred to herein as low dose contrast XAs or low dose XAs). Typically for example, the amount or concentration of contrast agent present in the blood vessels or vessels of interest when the at least one high dose XA is taken may be at least five times or at least ten times the amount or concentration present when the low dose XAs are taken. The second extraluminal images may be, in some examples, one or more subsequent images captured after the first extraluminal image is captured. In some examples, the second extraluminal images may take the form of a low dose contrast XA video or video stream which may be displayed in real time, as a live low dose XA. In some examples, the live low dose XA may be low dose XAs that are captured during an intravascular procedure, such as intravascular imaging, stent placement, etc. In some examples, live low dose XAs may be referred to as fluoroscopy images.

The at least one high dose contrast XA may be used to generate a vessel map. The low dose contrast XAs may be taken during the delivery of an intravascular device, such as a balloon, stent, probe, or the like. Correlating, or maintaining a mapping between, the at least one high dose XA and the low dose XAs can be implemented using motion features, which are typically features which are visible or detectable within, or derivable from, both the high and low dose XAs. For example, the motion features can include at least one of a guide catheter tip, a distal endpoint of a working vessel, an optical flow at the guide catheter tip, or an optical flow at the distal endpoint of the working vessel.

The correlation or mapping then allows for the vessel map to be overlaid on the low dose XAs, or vice versa, to provide a physician with visualization of where the intravascular device is within the vessel or vessel tree in real time. For example, this may allow for the intravascular device to be tracked on the live low dose XA with reference to the vessel map created using the at least one high dose XA. In some examples, a position of the intravascular device may be determined based on the live low dose XAs and an indication of the position may be provided on both the low dose XAs and the vessel map determined using the at least one high dose XA. According to some examples, correlating or mapping between the at least one high dose XA and the low dose XAs may allow for a treatment zone, such as a stent or balloon landing zone, identified on the high dose XA to be transferred, or superimposed, on the low dose XAs.

According to some examples, during a percutaneous coronary intervention (PCI) workflow, the at least one high dose contrast XAs may form at least part of a high dose XA video which may contain both pre-contrast, low dose XAs captured before contrast agent has been released into the blood vessel, and post-contrast, high dose XAs captured once contrast agent has been released. The post-contrast, high dose XAs may provide a clear view of the vessel tree, including side branches that are not typically visible in the pre-contrast, low dose XAs. The post-contrast, high dose XAs may be used to generate a vessel map. A vessel of interest, such as a working vessel, may be identified based on an intravascular device such as a guide catheter and guide wire in the pre-contrast, low dose, XAs. The identified working vessel may then be used to correlate, or map between, low dose and high dose XAs captured during the delivery of a device within the vessel.

Such a correlation or mapping provides for particular physical positions, such as a position within the working vessel, as captured in a low dose XA to be mapped to the same physical positions, such as the same position within the working vessel, in a high dose XA or vessel map. In this way features such as position of an intravascular device as seen or detected in the low dose XAs can be stably depicted within the context of one or more high dose XAs or a corresponding vessel map, or features such as features of a vessel map determined using the one or more high dose XAs can be stably depicted within the context of one or more low dose XAs.

The working vessel may be identified using one or more artificial intelligence (AI) models, such as machine learning (ML) models, that use the shape prior, e.g., the prediction of the location of the guide wire and/or guide catheter in the previous frame(s), to predict the location of the guide wire and/or guide catheter in the current frame. The AI model may be a spatial temporal model. The AI model may be trained based on annotated XAs. The annotations may be, in some examples, a set of line strips that follow the trajectory of the guide catheter and guide wire in pre-contrast XAs.

Motion features, which are typically features which are visible or detectable within, or derivable from, both the high and low dose XAs, may be used to correlate, or determine a mapping between, the high and low dose XAs to provide for dynamic visualization during the delivery of a device within a vessel. Because of ongoing movement of the blood vessels during capture of the low dose XAs, for example due to heart movement if the blood vessels are within cardiac tissue, this correlation or mapping may continuously or rapidly change, albeit on a repeated cycle such as that caused by a heartbeat, and the motion features of the low dose XAs may then be used to continuously update the correlation or mapping. The motion features may be identified in the high and low dose XAs. The motion features may be determined on a vessel level, vessel region of interest level, and a vessel pixel level to characterize the motion phase of the high and low dose XAs. The motion features may be used to perform an initial search to correlate high and low dose XAs having substantially the same temporal phase. A normalized cross correlation may be performed on pairs of motion features in the high and low dose XAs. For a given motion feature pair, samples of the low dose XA feature that are within a predetermined time window may be identified. The time window may be compared with all windows of substantially the same time length from the corresponding high dose XA feature. A maximum correlation coefficient may be determined based on the more correlated pair of high and low dose motion features. In some examples, an initial time shift may be determined. The initial time shift may be filtered using an online update. The online update may use a filter, such as a linear Kalman filter, to predict a time shift that would most likely match the most recently obtained low dose XA with the next available high dose XA.

The high and low dose XAs may be correlated with, or mapped between each other, based on the temporal correlation of motion features, without using any external sensor information. In some examples, in addition to or as an alternative of, the high and low dose XAs may be correlated using spatial landmarks.

According to some examples, the intravascular device may include one more markers, such as radiopaque markers, that are visible on low dose XAs. The markers may be detected and used to track the position of the intravascular device with respect to the vessel map generated based on the high dose XA. Tracking the device markers may allow a physician to determine whether the device is nearing and/or reached a treatment zone that was identified on the vessel map. In some examples, the treatment zone identified on the vessel may be reflected on the live low dose XA based on the correlation of the low and high dose XAs. By transferring annotations generated on the vessel map, e.g., the high dose XA, to the live low dose XAs, a physician may be able to dynamically visualize and track the position of the device with respect to the vessel morphology during the delivery of the device.

One aspect of the technology is generally directed to a computer implemented method (for example implemented using one or more suitable computer processors), comprising: receiving at least one first extraluminal image; receiving second extraluminal images captured during delivery of an intravascular device; detecting motion features in the at least one first extraluminal image and the second extraluminal images; correlating, based the detected motion features, the at least one first extraluminal image and the second extraluminal images; and providing for output, real-time visualization of a position of the intravascular device on the at least one first extraluminal image or one of the second extraluminal images including the intravascular device. The method may also comprise displaying, on a suitable display device such as a computer monitor, the output real-time visualization.

The at least one first extraluminal image may be a high dose contrast x-ray angiogram. The second extraluminal images may be low dose contrast x-ray angiograms. The intravascular device may be at least one of a stent delivery device, a balloon device, an intravascular imaging probe, a vessel prep device, or a pressure wire. The method may further comprise generating, by the one or more processors based on the at least one first extraluminal image, a vessel map.

The method may further comprise automatically detecting, by the one or more processors using a AI model, a working vessel. Training the AI model to automatically detect the working vessel may comprise: annotating pre-contrast extraluminal images as line strips following a trajectory of at least one of a guide wire or a guide catheter, labeling a path of the working vessel in post-contrast extraluminal images, providing the annotated pre-contrast extraluminal images and labeled post-contrast extraluminal images as training data to the AI model, and training the AI model to predict a working vessel trajectory. The method may further comprise receiving, by the one or more processors as input into the AI model at least one pre-contrast extraluminal image, training, by the one or more processors augmenting high-dose extraluminal images into low-dose extraluminal images as input, the AI model to segment at least one of a guide catheter, guide wire, stent marker, or balloon marker on the low-dose extraluminal images, detecting, by the one or more processors executing the AI model and based on the at least one pre-contrast extraluminal image, a guide wire of the intravascular device, propagating, by the one or more processors on a frame by frame basis executing the AI model and based on the detected guide wire, wire information, and automatically predicting, by the one or more processors executing the AI model based on the propagated wire information, a working vessel trajectory.

The motion features may include at least one of a guide catheter tip, a distal endpoint of a working vessel, an optical flow at the guide catheter tip, or an optical flow at the distal endpoint of the working vessel. Detecting the motion features may further comprise automatically detecting, by the one or more processors executing a AI model, the working vessel. Correlating the at least one first extraluminal image and the second extraluminal images may comprise: determining, by the one or more processors, vessel level motion, determining, by the one or more processors, wire tip level motion, and determining, by the one or more processors, vessel pixel level motion. Determining the vessel level motion may further comprise determining, by the one or more processors, a two-dimensional translation vector. The two-dimensional translation vector may correspond to two-dimensional translation at an n-th frame with respect to a first frame. Determining the wire tip level motion may comprise determining, by the one or more processors, absolute spatial information and relative spatial information. The absolute spatial information may correspond to coordinates and the relative spatial information corresponds to optical flow between adjacent image frames. Determining vessel pixel level motion may comprise determining, by the one or more processors, absolute spatial information and relative spatial information for a plurality of points on a working vessel. The absolute spatial information may correspond to coordinates and the relative spatial information corresponds to optical flow between adjacent image frames. The method may further comprise determining, based on the detected motion features, a heartbeat period of a patient.

The method may further comprise determining, by the one or more processors based on the detected motion features, a spatial-temporal phase match between the at least one first extraluminal image and at least one of the second extraluminal images. The method may further comprise resampling, by the one or more processors, the detected motion features at a common frame rate, determining, by the one or more processors, a maximum correlation coefficient for pairs of motion features in the at least one first extraluminal image and the second extraluminal images, and determining, by the one or more processors based on the maximum correlation coefficient, a time shift. The method may further comprise determining, by the one or more processors, a drift between detected motion features in the at least one first extraluminal image, and adjusting, by the one or more processors based on the determined drift, the time shift. The method may further comprise iteratively predicting, by the one or more processors, the time shift; and updating, by the one or more processors based on the iteratively predicted time shift, the time shift to a corrected time shift.

The method may further comprise tuning, by the one or more processors, the spatial-temporal phase match. Tuning the spatial-temporal phase match may comprise identifying, by the one or more processors, another first extraluminal image different than the at least one of the first extraluminal images and tuning, by the one or more processors based on the other first extraluminal image, the spatial-temporal phase match. The method may further comprise updating, by the one or more processors based on the tuning, the real-time visualization to include the other first extraluminal image.

The method may further comprise detecting, by the one or more processors, the intravascular device. Detecting the intravascular device in the second extraluminal images may further include executing a AI model. The method may further comprise training the AI model, wherein training the AI model comprises providing as input to the AI model a co-registration dataset comprising a plurality of intraluminal images and extraluminal images, wherein the plurality of intraluminal and extraluminal images are annotated images; and training the AI model to predict a position of the intravascular device. The annotated images include annotations identifying one or more intravascular device markers. The method may further comprise detecting, by the one or more processors, an optical flow of the intravascular device, determining, by the one or more processors based on the detected optical flow, a position of the intravascular device in a first frame of the second extraluminal images, and predicting, by the one or more processors based on the detected optical flow, the position of the intravascular device in a subsequent frame of the second extraluminal images.

The method may further comprise providing for output, by the one or more processors, a treatment zone on at least one of the second extraluminal images or the at least one first extraluminal image. The treatment zone is at least one of a treatment device landing zone, a stent landing zone, a balloon device zone, a vessel prep device zone, or a lesion related zone. The lesion related zone may be at least one of calcification frames, lipid frames, or dissected frames, and the lesion related zone may be identified from another imaging modality and co-registered, by the one or processors, to the first extraluminal image. The other imaging modality may be an intravascular imaging modality.

The method may further comprise receiving, by the one or more processors, annotations of the at least one first extraluminal image or the second extraluminal images. When receiving the annotations the method may further comprises receiving, by the one or more processors, one or more inputs from a user corresponding to the annotations, or automatically determining, by the one or more processors based on vessel data, the annotations. The annotations may include one or more of a plaque burden, fractional flow reserve (“FFR”) measurements at one or more locations along the vessel, calcium angles, EEL detections, calcium detections, proximal frames, distal frames, EEL-based metrics, stent decisions, scores, recommendations for debulking, recommendations for subsequent procedures, stent placement zone, treatment device landing zone, balloon device zone, vessel prep device zone, or lesion related zone. The method may further comprise updating, by the one or more processors based on the received annotations, a second one of the at least one first extraluminal image or the second extraluminal images.

The method may further comprise automatically capturing, by the one or more processors, a screen capture of the real-time visualization of the position of the intravascular device. The screen capture may be automatically captured when the intravascular device is within a threshold distance of a region of interest. The region of interest may be a treatment zone. The treatment zone may be at least one of a treatment device landing zone, a balloon device zone, a vessel prep device zone, or a lesion related zone. Determining the threshold distance may comprise at least one of determining, by the one or more processors, a number of pixels between an outer boundary of the region of interest and at least one detected marker on the intravascular device, or determining, by the one or more processors, a spatial distance between the at least one detected marker on the intravascular device and the region of interest. The spatial distance may be a Euclidean distance, a geodesic distance, etc.

The method may further comprise automatically zooming, by the one or more processors, a portion of the real-time visualization. The automatic zooming may focus on the portion of the real-time visualization when the intravascular device is within a threshold distance of a region of interest. The portion of the real-time visualization may correspond to the region of interest. The region of interest may be a treatment zone. The treatment zone may be at least one of a treatment device landing zone, a balloon device zone, a vessel prep device zone, or a lesion related zone. Determining the threshold distance may comprise at least one of determining, by the one or more processors, a number of pixels between an outer boundary of the region of interest and at least one detected marker on the intravascular device, or determining, by the one or more processors, a spatial distance between the at least one detected marker on the intravascular device and the region of interest. The spatial distance may be, for example, a Euclidean distance or a geodesic distance. In some examples, the automatic zooming may further comprise sorting, by the one or more processors, pixel values of the real-time visualization by their intensity, normalizing pixel intensity values lower than a predetermined threshold, applying a median filter to the normalized pixel intensity values of the real-time visualization.

Another aspect of the disclosure is directed to a system comprising one or more processors. The one or more processors may be configured to receive at least one first extraluminal image, receive second extraluminal images captured during delivery of an intravascular device, detect motion features in the at least one first extraluminal image and the second extraluminal images, correlate, based the detected motion features, the at least one first extraluminal image and the second extraluminal images, and provide for output real-time visualization of a position of the intravascular device on the at least one first extraluminal image or one of the second extraluminal images including the intravascular device.

Yet another aspect of the disclosure is directed to a non-transitory computer readable medium, which when executed by one or more processors cause the one or more processors to receive at least one first extraluminal image, receive second extraluminal images captured during delivery of an intravascular device, detect motion features in the at least one first extraluminal image and the second extraluminal images, correlate, based the detected motion features, the at least one first extraluminal image and the second extraluminal images, and provide for output real-time visualization of a position of the intravascular device on the at least one first extraluminal image or one of the second extraluminal images including the intravascular device.

One aspects of the technology relates to a method comprising receiving extraluminal images captured during the delivery of an intravascular device, wherein the intravascular device has a radio-opaque marker, detecting a plurality of device marker candidates corresponds to the radio-opaque marker of the intravascular device, automatically detecting, using an artificial intelligence (AI) model, a working vessel using a plurality of virtual boxes and at least one of the plurality of device marker candidates. The at least one of the plurality of virtual boxes contains the at least one of the plurality of device marker candidates. The method further comprises selecting, by the one or more processors, at least one of the plurality of virtual boxes that includes a region of interest of the working vessel that contains the at least one of the plurality of device markers.

In some examples, the method may further comprise predicting center points of each of the plurality of virtual boxes, pairing the detected device markers with the nearest of the plurality of virtual boxes, filtering the device marker candidates that are beyond the boundaries of the plurality of virtual boxes, updating center points of each of the plurality of virtual boxes using the filtered device marker candidate points, determining the displacement of the predicted center points and updated center points and repositioning, based on the determined displacement, the plurality of virtual boxes. The updating the center point may include approximating the center point of the box using the device marker candidate. The one or more device marker candidate may be within the at least one of the plurality of boxes.

In some examples, the AI model may be trained using annotations on high dose angiographs. In some examples, the extraluminal images may be live x-ray angiographs or fluoroscopy images.

In some examples, the method may further comprise tracking the region of interest during a percutaneous coronary intervention procedure. The method may further include enhancing the region of interest using local contrast stretching by selectively enhancing the local contrast between the detected device marker candidate and surrounding regions. In some examples, the method may further comprise automatically zooming, by the one or more processors, the region of interest. The automatic zooming may comprise sorting, by the one ore more processors, pixel values of the real-time visualization by their intensity, normalizing pixel intensity values lower than a predetermined threshold, and applying a median filter to the normalized pixel intensity values of the real time visualization.

In another aspect of the disclosure is directed to a system comprising one or more processors configured to receive extraluminal images captured during the delivery of an intravascular device, wherein the intravascular device has a radio-opaque marker, detect a plurality of device marker candidates, wherein at least one of the device marker candidates corresponds to the radio-opaque marker of the intravascular device, automatically detect, using an artificial intelligence model, a working vessel using a plurality of virtual boxes and at least one of the plurality of device marker candidates, wherein at least one of the plurality of virtual boxes contains the at least one of the plurality of device marker candidates, and select at least one of the plurality of virtual boxes that includes a region of interest of the working vessel that contains the at least one of the plurality of device markers.

In yet another aspect of the disclosure is directed to a non-transitory computer-readable medium storing instructions, which when executed by one or more processors causes the one or more processors receive extraluminal images captured during the delivery of an intravascular device, wherein the intravascular device has a radio-opaque marker, detect a plurality of device marker candidates, wherein at least one of the device marker candidates corresponds to the radio-opaque marker of the intravascular device, automatically detect, using an artificial intelligence model, a working vessel using a plurality of virtual boxes and at least one of the plurality of device marker candidates, wherein at least one of the plurality of virtual boxes contains the at least one of the plurality of device marker candidates, and select at least one of the plurality of virtual boxes that includes a region of interest of the working vessel that contains the at least one of the plurality of device markers.

### BRIEF DESCRIPTION OF THE DRAWINGS

FIG. 1 is an example data collection system for collecting intravascular and extravascular data, according to aspects of the disclosure.

FIG. 2 is a flow diagram illustrating a method of dynamic visualization of an intravascular device for example within the system of FIG. 1, according to aspects of the disclosure.

FIG. 3A is a flow diagram illustrating a method of identifying a working vessel and device markers, according to aspects of the disclosure, which may be implemented within the data collection system of FIG. 1, for implementing the method of dynamic visualization of FIG. 2.

FIG. 3B shows example skeleton frames used in the method of FIG. 3A, according to aspects of the disclosure.

FIG. 3C is a flow chart illustrating a method of alignment for marker display used in the method of FIG. 3A, according to aspects of the disclosure.

FIG. 4 is a flow chart illustrating a method of generating the shape prior according to aspects of the disclosure, which may be implemented within the data collection system of FIG. 1, for implementing the method of dynamic visualization of FIG. 2.

FIG. 5A is a spatial temporal encoder decoder segmentation network according to aspects of the disclosure, which may be implemented within the data collection system of FIG. 1, for implementing the method of dynamic visualization of FIG. 2.

FIG. 5B shows annotations for training a AI model used in the network of FIG. 5A, according to aspects of the disclosure.

FIG. 6 is a flow chart illustrating a method of motion phase matching, which may be implemented within the data collection system of FIG. 1, for implementing the method of dynamic visualization of FIG. 2 according to aspects of the disclosure.

FIG. 7A is a diagram for determining motion features, which may be implemented within the data collection system of FIG. 1, for implementing the method of dynamic visualization of FIG. 2 according to aspects of the disclosure.

FIG. 7B shows a graphical representation of period patterns of motion features as described in the diagram of FIG. 7A, according to aspects of the disclosure.

FIG. 8 is a flow diagram illustrating a method of temporal phase matching, which may be implemented within the data collection system of FIG. 1, for implementing the method of dynamic visualization of FIG. 2 according to aspects of the disclosure.

FIG. 9 is a flow diagram illustrating a method of the initial search for the temporal phase match, which may be implemented within the data collection system of FIG. 1, for implementing the method of dynamic visualization of FIG. 2 according to aspects of the disclosure.

FIG. 10 is a flow diagram illustrating a method of updating the initial search for the temporal phase match, which may be implemented within the data collection system of FIG. 1, for implementing the method of dynamic visualization of FIG. 2 according to aspects of the disclosure.

FIG. 11 is a flow diagram illustrating a method of determining a heartbeat period, which may be implemented within the data collection system of FIG. 1, for implementing the method of dynamic visualization of FIG. 2 according to aspects of the disclosure.

FIG. 12 is a flow diagram illustrating a method of detecting and tracking device markers, which may be implemented within the data collection system of FIG. 1, for implementing the method of dynamic visualization of FIG. 2 according to aspects of the disclosure.

FIG. 13 is a flow diagram illustrating a method of determining marker positions, which may be implemented within the data collection system of FIG. 1, for implementing the method of dynamic visualization of FIG. 2 according to aspects of the disclosure.

FIG. 14 is an example of marker detection and tracking, which may be implemented within the data collection system of FIG. 1, for implementing the method of dynamic visualization of FIG. 2 according to aspects of the disclosure.

FIG. 15 is an example of identifying a landing zone, which may be implemented within the data collection system of FIG. 1, for implementing the method of dynamic visualization of FIG. 2 according to aspects of the disclosure.

FIG. 16 is a flow diagram illustrating a method of dynamically visualizing the delivery of a device, which may be implemented within the data collection system of FIG. 1, for implementing the method of dynamic visualization of FIG. 2 according to aspects of the disclosure.

FIG. 17 is a flow diagram illustrating a method of dynamically visualizing the delivery of a device, which may be implemented within the data collection system of FIG. 1, for implementing the method of dynamic visualization of FIG. 2 according to aspects of the disclosure.

FIG. 18 is an example of tuning the temporal synchronization of extraluminal images, which may be implemented within the data collection system of FIG. 1, for implementing the method of dynamic visualization of FIG. 2 according to aspects of the disclosure.

FIG. 19 is another example of tuning the temporal synchronization of extraluminal images, which may be implemented within the data collection system of FIG. 1, for implementing the method of dynamic visualization of FIG. 2 according to aspects of the disclosure.

FIG. 20 is another example of tuning the temporal synchronization of extraluminal images, which may be implemented within the data collection system of FIG. 1, for implementing the method of dynamic visualization of FIG. 2 according to aspects of the disclosure.

FIG. 21 is an example of a real-time state transition for dynamically visualizing the delivery of a device, which may be implemented within the data collection system of FIG. 1, for implementing the method of dynamic visualization of FIG. 2 according to aspects of the disclosure.

FIG. 22 illustrates determining device marker candidates from the extraluminal image and the shape prior, which may be implemented within the data collection system of FIG. 1, for implementing the method of dynamic visualization of FIG. 2.

FIG. 23 are example annotations for training an artificial intelligence model, which may be implemented within the data collection system of FIG. 1, for implementing the method of dynamic visualization of FIG. 2 according to aspects of the disclosure.

FIG. 24 is an illustration of annotations overlaid on XA images, which may be implemented within the data collection system of FIG. 1, for implementing the method of dynamic visualization of FIG. 2 according to aspects of the disclosure.

FIG. 25 is an illustration of a method of defining regions of interest and propagating the regions of interest, which may be implemented within the data collection system of FIG. 1, for implementing the method of dynamic visualization of FIG. 2 according to aspects of the disclosure.

FIG. 26 is a flow diagram illustrating a method of determining device marker regions of interest, which may be implemented within the data collection system of FIG. 1, for implementing the method of dynamic visualization of FIG. 2 according to aspects of the disclosure.

FIG. 27A is a real-time local contrast stretching to enhance device marker appearance in extraluminal images, which may be implemented within the data collection system of FIG. 1, for implementing the method of dynamic visualization of FIG. 2 according to aspects of the disclosure.

FIG. 27B is another example real-time local contrast stretching to enhance device marker appearance in extraluminal images, which may be implemented within the data collection system of FIG. 1, for implementing the method of dynamic visualization of FIG. 2 according to aspects of the disclosure.

FIG. 27C is another example real-time local contrast stretching to enhance device marker appearance in extraluminal images, which may be implemented within the data collection system of FIG. 1, for implementing the method of dynamic visualization of FIG. 2 according to aspects of the disclosure.

### DETAILED DESCRIPTION

The technology is generally directed to dynamically visualizing an intravascular device within a blood vessel by correlating or mapping between extraluminal images. The extraluminal images may be correlated based on motion features visible or detectable within the images themselves, without the use of other sensors, such as readings from an electrocardiogram, and without the use of time stamps. The extraluminal images may include pre-contrast, high dose post-contrast, and subsequent low dose x-ray angiograms (“XA”). For example, a high dose XA may be used to generate a blood vessel map, pre-contrast XAs may be used to identify a vessel of interest or working vessel, and low dose XAs may be taken during the delivery of the device and used to track the position of the intravascular device.

High dose contrast XA images use more contrast agent during imaging as compared to low dose contrast XA images. According to some examples, a high dose XA may use 8-10 cc of a contrast agent, where a low dose XA may use 2-3 cc of the contrast agent. In some examples, a high dose XA may use at least three to five times more contrast than a low dose XA. In yet another example, a high dose XA may use at least five or more time more contrast than a low dose XA. The high dose XA images may cause the patient to have a higher radiation dose than low dose contrast XA images. High dose XA images may have longer imaging times as compared to low dose contrast XA images. The high dose XA images may have better diagnostic quality due to the increased contrast and/or higher radiation dose as compared to the low dose XA images having a lower contrast and/or lower radiation dose. As used herein, high dose contrast XA may be interchangeable with high dose XA and low dose contrast XAs may be interchangeable with low dose XA.

According to some examples, a guide wire and/or guide catheter used for delivering the device may be used to identify the working vessel. The guide wire and guide catheter may be visible in pre-contrast XAs. For example, the system can integrate artificial intelligence (“AI”) to identify the working vessel. As an example, the AI may be a AI (“ML”) model trained to segment XAs to predict output classes, including guide catheter, guide wire, catheter marker, and background. The multi-label segmentation output by the ML model may be converted into a binary mask that illustrates the working vessel region as a shape prior to the subsequent XAs. According to some examples, the initial shape prior may be obtained from a pre-contrast XA. The initial shape prior determined based on the pre-contrast XA may be used to segment the working vessel in subsequent high dose XAs. According to some examples, the AI may include a second ML model that is trained to identify the working vessel based on the shape prior determined by the first ML model. The second ML model may be a spatial temporal model. The second ML model may receive, as input, the current XA frame plus prediction information of the previous XA frames. The prediction information may be, for example, the prediction of the location of the working vessel in the given XA frame. In some examples, the second ML model may receive, as an additional input, the shape prior of the previous frame(s). The second ML model may provide, as output, the identified working vessel. According to some examples, AI may be used to segment the guide catheter and wire tip such that the locations of ending points of guide catheter and guide wire tip may be automatically detected based on the segmentation results. The AI may include other ML models similar to the first and/or second ML models.

A motion feature bank may be generated based on motion features detected in the high dose and low dose XAs. The feature bank may provide motion information from various perspectives and may be used for phase matching the low dose and high dose XAs for a given patient. The motion features may be used to perform an initial phase matching of the low dose and high dose XAs. The motion features may include a guide catheter tip, a distal endpoint of a working vessel, an optical flow at the guide catheter tip, and/or an optical flow at the distal endpoint of the working vessel. The initial phase matching may be an initial temporal phase matching. The high and low dose XAs may be temporally matched without the use of EKG or other physiological signals that measure heartbeat or breath patterns of a patient or time stamps. Rather, the temporal phase matching may be based on image motion features.

According to some examples, an update may be performed to iteratively filter the initial time shift estimate. The initial time shift estimate may be updated during an online update. For example, a filter, such as a linear Kalman filter, may be used to predict a time shift based on the more current low dose XA and the next available high dose XA. The predicted time shift may be used to cross-correlate the low and high dose XAs to determine whether the filter should be updated. The time shift determined by the online update may be used to correlate the low and high dose XAs. In some examples, a spatial fitness between spatial landmarks, such as the guide catheter, wire tip, device, or the like, identified in the high and low dose XAs may be used to evaluate and/or refine the phase matching performed using the motion features.

According to some examples, the motion features may be used to derive the heartbeat period of the patient. For example, an auto-correlation function may be applied to the motion features. The heartbeat period may be detected based on voting and non-maximum suppression.

To provide a visualization of the device during the delivery and/or treatment stage, the markers on the device may be detected and tracked. The device may include one or more radiopaque markers that are able to be detected using the high and/or low dose XA. The device may be detected and tracked by iteratively estimating the position of the marker in the XAs. In some examples, the motion of the marker within the sampling period is estimated by an optical flow method. The motion estimates may be used to predict the position of the marker in the most recent XA.

Correlating live low dose XAs with high dose XAs may reduce the amount of contrast required during PCI. For example, by identifying motion features in both the low dose and high dose XAs, the low dose XAs may be temporally and spatially correlated such that the position of the device can be tracked real time in low dose XAs relative to a vessel map generated from a high dose XA.

In some examples, correlative live low dose XAs with high dose XAs using motion features may reduce the number of sensors and/or the amount of equipment required to co-register low dose and high dose XAs. For example, by using motion features that can be detected in the low and high dose XAs, there is no longer a need to synchronize the collection of images, time stamps, or the like. Further, the use of an EKG to co-register the images based on a heartbeat cycle is negated. According to some examples, by correlating the low and high dose XAs using motion features, the amount of contrast needed during a procedure may be reduced. For example, a physician will be able to dynamically visualize the location of the device within the vessel based on the correlated low and high dose XA without having to inject additional contrast to determine where the device is within the vessel. Rather, the dynamic visualization allows for a physician to see, in real time, the location of the device on the vessel map without additional contrast.

According to some examples, by using AI to correlate the low and high dose XAs to provide dynamic visualization of the delivery of a device within a vessel, the amount of computational resources required to produce the dynamic visualization is reduced. For example, the AI may include one or more AI models. The use of one or more AI models to correlate the high and low dose XAs may reduce the amount of memory and processing power required to dynamically visualize the device as it is being delivered within the vessel.

### Example System

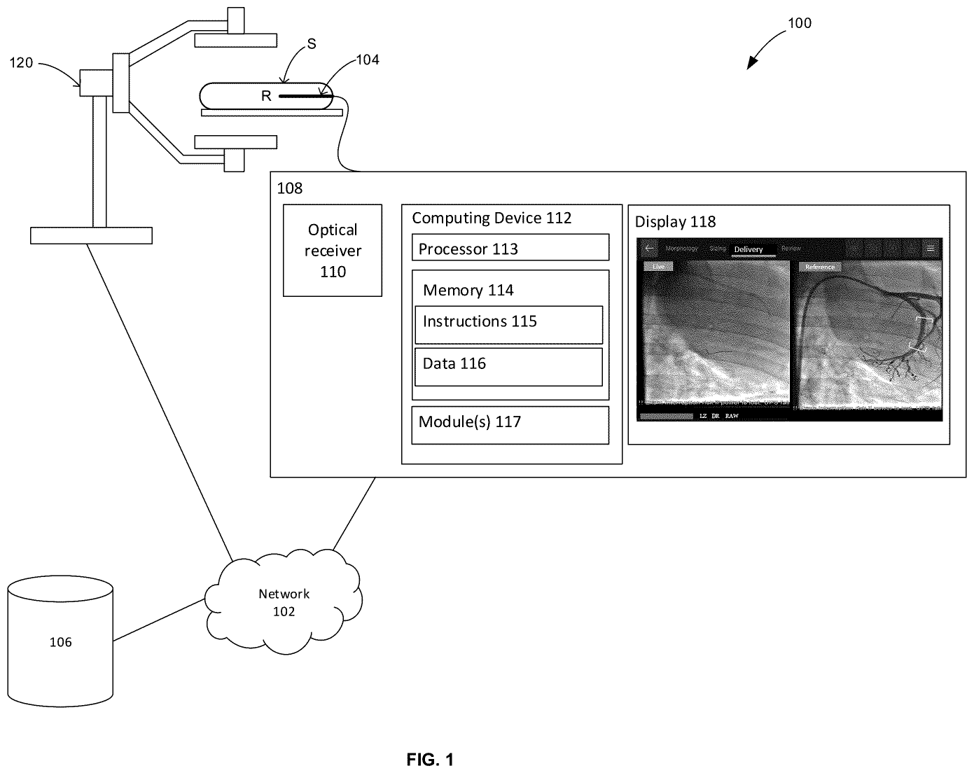

FIG. 1 illustrates a data collection system 100 for use in collecting intravascular and extravascular data. The system 100 may include a non-invasive imaging system 120, an intravascular device 104, a storage device 106, subsystem 108, and network 102. The subsystem 108 may include an optical receiver 110, computing device 112, and display 118.

The non-invasive imaging system 120 may be, for example, a nuclear magnetic resonance, x-ray, computer aided tomography, or other suitable non-invasive imaging technology. In particular, the non-invasive imaging system may be an angiography system configured to generate pre-contrast X-ray angiograms (XAs) before a radio opaque contrast agent has been used, high dose contrast XAs taken while a radio-opaque contrast agent is present in one or more blood vessels of interest, and low dose contrast XAs taken when little or no contrast agent is present. The high dose contrast XAs may, in some examples, be cine images, and the low dose contrast XAs may be fluoroscopic images. For example, the angiography system 120 may include a fluoroscopy system. The angiography system 120 may be configured to noninvasively image the subject S such that frames of angiography data, typically in the form of frames of image data, are generated. According to some examples, the x-ray imaging may occur while a device 104 is being delivered via the vessel such that a blood vessel in region R of subject S is imaged using angiography. In some examples, high dose XAs may be generated before the intravascular device 104 is delivered via the vessel, while the device is being delivered, and/or after the device has been delivered. The imaging results of a non-invasive scan may be provided for output on display 118. The high dose and low dose XAs may be correlated such that the device and/or treatment zone may be dynamically visualized during a procedure. As discussed above and herein, the non-invasive imaging system may be an angiography system. However, the non-invasive imaging system 120 may use various other imaging technologies and, therefore, references to the non-invasive imaging system 120 being an angiography system are not intended to be limiting.

The non-invasive imaging system 120 may be in communication with a storage device 106 via network 102. In some examples, the imaging system 120 may be in direct communication with storage device 106, e.g., without having to be connected via network 102. The storage device 106 may be a workstation or server. The data collected and/or generated by imaging system 120 may be storage and managed by the storage system 106. In some examples, a subsystem, a server or workstation handle the functions of storage device 106. According to some examples, the imaging system 120 may generate electromagnetic radiation, such as x-rays. The imaging system 120 may, in some examples, receive such radiation after passing through the subject S. In turn, data storage 106 may use the signals from the imaging system 120 to image one or more regions of the subject S including region R. In some examples, storage device 106 may be integrated with imaging device 120.

The region of interest R may be a subset of the vascular or peripherally vascular system, such as a particular blood vessel. The region R may be a region for the delivery of a device 104. The device may be, for example, a stent, balloon, or the like. In some examples, the device 104 may be an intravascular device, such as an optical coherence tomography (“OCT”) probe, an intravascular ultrasound (“IVUS”) catheter, micro-OCT probe, near infrared spectroscopy (NIRS) sensor, optical frequency domain imaging (“OFDI”), or any other device that can be used to image a blood vessel. In some examples, the device 104 may be a pressure wire, a flow meter, etc. The device 104 may include a device tip, one or more radiopaque markers, an optical fiber, a torque wire, or the like. Additionally, the device tip may include one or more data collecting subsystems such as an optical beam director, an acoustic beam director, a pressure detector sensor, other transducers or detectors, and combinations of the foregoing.

In examples where the device 104 includes an optical beam director, the optical fiber may be in optical communication with the device 104 and/or with the beam director. The torque wire may define a bore in which an optical fiber is disposed. According to some examples, the device 104 may include the sheath such as a polymer sheath (not shown) which forms part of a catheter. The optical fiber, which in the context of an intravascular system is a portion of the sample arm of an interferometer, may be optically coupled to subsystem 108 and/or a patient interface unit (PIU).

A guide wire, not shown, may be used to introduce the device 104 into the blood vessel. In examples where the device 104 is an intravascular data collection device, the device 104 may be introduced and pulled back along a length of a blood vessel while collecting data. In examples where the device 104 is a treatment device, such as a device to deliver a balloon or stent, the device 104 may be introduced to the vessel and guided to a treatment zone.

The device 104 may be connected to a subsystem 108. According to some examples, the device 104 may be connected to subsystem 108 via an optical fiber. The subsystem 108 may include a light source, such as a laser, an interferometer having a sample arm and a reference arm, various optical paths, a clock generator, photodiodes, and other OCT, IVUS, micro-OCT, NIRS, and/or pressure wire components. The device 104 may be connected to an optical receiver 110. According to some examples, the optical receiver 110 may be a balanced photodiode based system. The optical receiver 110 may be configured to receive light collected by the probe 104. The device 104 may be coupled to the optical receiver 110 via a wired or wireless connection.

The system 100 may further include, or be configured to receive data from, an non-invasive imaging system 120. The non-invasive imaging system 120 may be, for example, an imaging system based on angiography, fluoroscopy, x-ray, nuclear magnetic resonance, computer aided tomography, etc. non-invasive imaging system 120 may be configured to noninvasively image the blood vessel 102. According to some examples, the non-invasive imaging system 120 may obtain one or more images before, during, and/or after a pullback of the data collection probe 104. Non-invasive imaging system 120 may be used to image a patient such that diagnostic decisions can be made and various possible treatment options such as stent placement can be carried out. These and other imaging systems can be used to image a patient externally or internally to obtain raw data, which can include various types of image data.

The non-invasive imaging system 120 may be in communication with subsystem 108. According to some examples, the non-invasive imaging system 120 may be wirelessly coupled to subsystem 108 via network 102. For example, the non-invasive imaging system 120 may be wirelessly coupled to subsystem 108 via a communications interface, such as Wi-Fi or Bluetooth. In some examples, the non-invasive imaging system 120 may be in communication with subsystem 108 via a wire, such as an optical fiber. In yet another example, external imaging device 120 may be indirectly communicatively coupled to subsystem 108 or computing device 112. For example, the non-invasive imaging device 120 may be coupled to a separate computing device (not shown) that is in communication with computing device 112. As another example, data from the imaging system 120 may be transferred to the computing device 112 using a computer-readable storage medium, from storage device 106 via network 102, or the like.

The subsystem 108 may include a computing device 112. One or more steps may be performed automatically or without user input to navigate images, input information, select and/or interact with an input, etc. In some examples, one or more steps may be performed based on receiving a user input by mouse clicks, a keyboard, touch screen, verbal commands, etc. The computing device 112 may include one or more processors 113, memory 114, instructions 115, data 116, and one or more modules 117.

The one or more processors 113 may be any conventional processors, such as commercially available microprocessors. Alternatively, the one or more processors may be a dedicated device such as an application specific integrated circuit (ASIC) or other hardware-based processor. Although FIG. 1 functionally illustrates the processor, memory, and other elements of device 112 as being within the same block, it will be understood by those of ordinary skill in the art that the processor, computing device, or memory may actually include multiple processors, computing devices, or memories that may or may not be stored within the same physical housing. Similarly, the memory may be a hard drive or other storage media located in a housing different from that of device 112. Accordingly, references to a processor or computing device will be understood to include references to a collection of processors or computing devices or memories that may or may not operate in parallel.

Memory 114 may store information that is accessible by the processors, including instructions 115 that may be executed by the processors 113, and data 116. The memory 114 may be a type of memory operative to store information accessible by the processors 113, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions 115 and data 116 are stored on different types of media.

Memory 114 may be retrieved, stored or modified by processors 113 in accordance with the instructions 115. For instance, although the present disclosure is not limited by a particular data structure, the data 116 may be stored in computer registers, in a relational database as a table having a plurality of different fields and records, XML documents, or flat files. The data 116 may also be formatted in a computer-readable format such as, but not limited to, binary values, ASCII or Unicode. By further way of example only, the data 116 may be stored as bitmaps comprised of pixels that are stored in compressed or uncompressed, or various image formats (e.g., JPEG), vector-based formats (e.g., SVG) or computer instructions for drawing graphics. Moreover, the data 116 may comprise information sufficient to identify the relevant information, such as numbers, descriptive text, proprietary codes, pointers, references to data stored in other memories (including other network locations) or information that is used by a function to calculate the relevant data.

The instructions 115 can be any set of instructions to be executed directly, such as machine code, or indirectly, such as scripts, by the processor 113. In that regard, the terms “instructions,” “application,” “steps,” and “programs” can be used interchangeably herein. The instructions can be stored in object code format for direct processing by the processor, or in any other computing device language including scripts or collections of independent source code modules that are interpreted on demand or compiled in advance. Functions, methods and routines of the instructions are explained in more detail below.

According to some examples, the computing device 112 may include receive, either by a wired connection or via a wireless connection, data from the device 104. The data may include, for example, intravascular data including intravascular imaging data, pressure data, temperature data, flow data, or the like. The data received by the computing device 112 from imaging system 120, storage device 106, and/or device 104 may be used to dynamically visualize the device 104 on one or more extraluminal images. In some examples, where the device is an intravascular data collection device, the data received from the device may be used to determine plaque burden, fractional flow reserve (“FFR”) measurements at one or more locations along the vessel, calcium angles, EEL detections, calcium detections, proximal frames, distal frames, EEL-based metrics, stent/no stent decisions, scores, recommendations for debulking and other procedures, evidence based recommendations informed by automatic detection of regions/features of interest, stent planning, etc.

In examples, the device 104 is an intravascular data collection device, the data obtained by device 104 and/or imaging system 120 may processed by one or more modules to provide information about the blood vessel including lumen contours, vessel diameters, vessel cross-sectional areas, lumen area, EEL values, EEL diameters, EEL arcs, lesion locations, lesion size, plaque burdens, VFR, FFR, landing zones, treatment zones, virtual stents bounded by the landing zones or the like. According to some examples, the modules 117 may include a dynamic visualization module, an EEL detection module, a lumen detection module, a lesion detection module, and a co-registration module.

The dynamic visualization module may automatically correlate high dose and low dose XA such that a vessel map may be overlaid on the live low dose XA, a treatment zone identified on the high dose XA and/or vessel map may be overlaid on the live low dose XA, and/or the device 104 may be tracked on the live low dose XA with reference to the vessel map. The dynamic visualization module may correlate the high and low dose XA based on motion features detected in both the high and low dose XA. In some examples, the dynamic visualization module may usc AI, such as one or more AI (“ML”) models, to identify the working vessel, detect markers on the device, and track the motion of the device.

The EEL detection module may automatically detect and measure the EEL diameter of a given intravascular image frame taken during a pullback of a vessel. The lumen detection module may automatically detect and measure the lumen diameter of a given intravascular image frame taken during a pullback of the vessel. The lesion detection module may automatically detect lesions within the vessel based on vessel data obtained from the device 104 and/or imaging system 120. The side branch detection module may process the vessel data obtained from the device 104 and/or imaging system 120 to detect one or more side branches of the blood vessel.

The computing device 112 may be adapted to co-register vessel data obtained during a pullback of the device 104 with intravascular image and/or an extraluminal image. For example, the computing device 112 may be configured to receive and store extraluminal image data, such as image data generated by imaging system 120 and obtained by a frame grabber. The computing device 112 may be configured to receive and store intravascular image data, such as image data generated by device 104 and obtained by the frame grabber. In some examples, computing device 112 may access co-registration module to co-register the vessel data with the luminal image. The luminal image may be an extraluminal image, such as an angiograph, x-ray, or the like. The co-registration module may co-register intravascular data, such as an intravascular image, plaque burden, EEL measurement, lumen diameter measurements, pressure readings, virtual flow reserve (“VFR”), fractional flow reserve (“FFR”), resting full-cycle ration (“RFR”), flow rates, etc. with the extraluminal image. In some examples, the co-registration module may co-register intravascular data with an intraluminal image, such as an intraluminal image captured by an OCT probe, IVUS probe, micro-OCT probe, or the like.

In one example, the co-registration module may co-register intraluminal data captured during a pullback with one or more extraluminal images. For example, the extraluminal image frames may be pre-processed. Various matrices such as convolution matrices, Hessians, and others can be applied on a per pixel basis to change the intensity, remove, or otherwise modify a given angiography image frame. As discussed herein, the preprocessing stage may enhance, modify, and/or remove features of the extraluminal images to increase the accuracy, processing speed, success rate, and other properties of subsequent processing stages. A vessel centerline may be determined and/or calculated. In some examples, the vessel centerline may be superimposed or otherwise displayed relative to the pre-processed extraluminal image. According to some examples, the vessel centerline may represent a trajectory of the device 104, such as an intravascular device, through the blood vessel during a pullback. In some examples, the centerline may be referred to as a trace. Additionally or alternatively, marker bands or radiopaque markers may be detected in the extraluminal image frames. According to some examples, the extraluminal image frames and the data received by device 104 may be co-registered based on the determined location of the marker bands.

According to some examples, the modules may additionally or alternatively include a video processing software module, a preprocessing software module, an image file size reduction software module, a catheter removal software module, a shadow removal software module, a vessel enhancement software module, a blob enhancement software module, a Laplacian of Gaussian filter or transform software module, a guide wire detection software module, an anatomic feature detection software module, stationary marker detection software module, a background subtraction module, a Frangi vesselness software module, an image intensity sampling module, a moving marker software detection module, iterative centerline testing software module, a background subtraction software module, a morphological close operation software module, a feature tracking software module, a catheter detection software module, a bottom hat filter software module, a path detection software module, a Dijkstra software module, a Viterbi software module, fast marching method based software modules, a vessel centerline generation software module, a vessel centerline tracking module software module, a Hessian software module, an intensity sampling software module, a superposition of image intensity software module and other suitable software modules as described herein. According to some examples, the modules may include software such as preprocessing software, transforms, matrices, and other software-based components that are used to process image data or respond to patient triggers to facilitate co-registration of different types of image data by other software-based components or to otherwise perform annotation of image data to generate ground truths and other software, modules, and functions suitable for implementing various features of the disclosure. The modules can include lumen detection using a scan line based or image based approach, stent detection using a scan line based or image based approach, indicator generation, apposition bar generation for stent planning, guide wire shadow indicator to prevent confusion with dissention, side branches and missing data, and others.

In some examples, the modules may be configured to process the vessel data obtained by the device 104 and/or imaging system 120 using AI algorithms, artificial intelligence, or the like.

The subsystem 108 may include a display 118 for outputting content to a user. The display 118 may be integrated with the computing device 112, or it may be a standalone unit electronically coupled to the computing device 112. The display 118 may output intravascular data relating to one or more features detected in the blood vessel and/or obtained during a pullback. For example, the output may include, without limitation, cross-sectional scan data, longitudinal scans, three-dimensional representations generated based on intraluminal and/or extraluminal images, diameter graphs, image masks, lumen border, plaque sizes, plaque circumference, visual indicia of plaque location, visual indicia of risk posed to stent expansion, flow rate, suggested treatment zones, or the like. The display 118 may identify features with text, arrows, color coding, highlighting, contour lines, or other suitable human or machine-readable indicia.

According to some examples the display 118 may include a graphic user interface (“GUI”). The display 118 may be a touchscreen display in which a user can provide an input to navigate images, input information, select and/or interact with an input, etc. In some examples, the display 118 and/or computing device 112 may include an input device, such as a trackpad, mouse, keyboard, etc. that allows a user to navigate images, input information, select and/or interact with an input, etc.

In some examples, the information input by the user may be annotations. For example, the system may be configured to receive annotations to the one or more representations of the vessel. The annotations may be, in some examples, an indication of plaque burden, fractional flow reserve (“FFR”) measurements at one or more locations along the vessel, calcium angles, EEL detections, calcium detections, proximal frames, distal frames, EEL-based metrics, stent/no stent decisions, scores, recommendations for debulking and other procedures, evidence based recommendations informed by automatic detection of regions/features of interest, stent planning, etc. In some examples, the annotations maybe a treatment device landing zone, balloon device zone, vessel prep device zone, or lesion related zone. For example, the system may receive an input corresponding to a proximal and distal location along the vessel corresponding to a proximal and distal location of a treatment device landing zone, balloon device zone, vessel prep device zone, or lesion related zone.

According to some examples, the annotations may be automatically determined by the system. For example, the system may, based on vessel data, determine one or more of plaque burden, FFR measurements at one or more locations along the vessel, calcium angles, EEL detections, calcium detections, proximal frames, distal frames, EEL-based metrics, stent/no stent decisions, scores, recommendations for debulking and other procedures, evidence based recommendations informed by automatic detection of regions/features of interest, stent planning, a treatment device landing zone, balloon device landing zone, vessel prep device zone, lesion related zone, etc. The system may automatically provide the plaque burden, FFR measurements at one or more locations along the vessel, calcium angles, EEL detections, calcium detections, proximal frames, distal frames, EEL-based metrics, stent/no stent decisions, scores, recommendations for debulking and other procedures, evidence based recommendations informed by automatic detection of regions/features of interest, stent planning, a treatment device landing zone, balloon device landing zone, vessel prep device zone, lesion related zone, etc. for output as one or more annotations on at least one of the vessel representations.

In some examples, inputs received by the system corresponding to input information and/or annotations (collectively “annotations”) may be provided for output on display 118. Annotations received with respect to one vessel representation may be provided for display on the vessel representation in which the annotations were received. In some examples, the annotations may be provided for display on a plurality of vessel representations. For example, if the system receives annotations with respect to the high dose XA, the annotations may be provided for output on one or both the high dose XA and the low dose XA. In examples where there are additional representations, such as longitudinal representations, three-dimensional representations, or the like, the annotations may be provided for output on one, some, or all the representations. According to some examples, when the system receives updated annotations on at least one of the representations, the annotations on the other representations may be updated to correspond to the updated annotations.

The display 118 alone or in combination with computing device 112 may allow for toggling between one or more viewing modes in response to user inputs. For example, a user may be able to toggle between different intravascular data, images, etc. recorded during each of the pullbacks. In some examples, the user may be able to toggle between different representations, such as a longitudinal representation, a cross-sectional representation, a three-dimensional representation, intravascular images, color images, black and white images, live images, or the like.

In some examples, the display 118, alone or in combination with computing device 112, may present one or more menus as output to the physician, and the physician may provide input in response by selecting an item from the one or more menus. For example, the menu may allow a user to show or hide various features. As another example, there may be a menu for selecting blood vessel features to display.

The output may include one or more representations of the vessel. For example, the representations may include images data, longitudinal representations, three-dimensional representations, live representations, or the like. The image data may include, for example, pre-contrast, high dose contrast, and low dose contrast XA images obtained and/or generated by the imaging system 120. In some examples, the output may include cross-sectional images obtained by the device 104 during a pullback. In some examples, the output extraluminal images may include a vessel map generated based on high dose XA and live low dose XA images obtained during delivery of a device. The low dose XA may include an indication of the location of the device 104, an indication of the treatment zone originally identified on the high dose XA, or the like. In some examples, the output may include a longitudinal representation of the vessel, a graphical representation of the vessel data, a three-dimensional representation of the vessel and/or vessel data, or the like. The representations of the vessel may provide various viewing angles and section views. In some examples, EEL positions, diameters thereof, or other EEL based parameters may be output for display relative to angular measurements, detected calcium arcs, plaque burden, or the like.

In some examples, one or more visual representations of the images may include an indication of a lesion location, lesion severity, lesion length, or the like. Additionally or alternatively, the indication of the lesion may be color coded, where each color represents the severity, length, or other measurement related to the lesion.

In some examples, the output may include candidate treatment zones. The candidate treatment zone may be, for example, a candidate stent landing zone. For example, the output may include an indication corresponding to a candidate proximal landing zone for a stent and a candidate distal landing zone for a stent. The candidate treatment zone may be determined based on the determined plaque burden, lesion locations, lesion length, or the like. The indications may be provided on any of the vessel representations, e.g., the three-dimensional representation, the longitudinal representation, the graphical representation, image data such as the external images, etc.

According to some examples, the display 118 and/or computing device 112 may be configured to receive one or more inputs corresponding to a selection on one or more representations. For example, an input may be received corresponding to a selection of an image frame on the longitudinal representation. In response, the other representations provided for output may be updated to display a corresponding indication or image frame. For example, the extraluminal image may be updated to have an indication along the vessel corresponding to the location of the image frame selected in the longitudinal representation, a circumferential indication may be provided on a three-dimensional representation corresponding to the location of the image frame selected in the longitudinal representation, the cross-sectional image frame may be updated to correspond to the image frame selected in the longitudinal representation, etc. In some examples, the vessel data associated with the selected location may be updated and provided for display.

### Dynamic Visualization

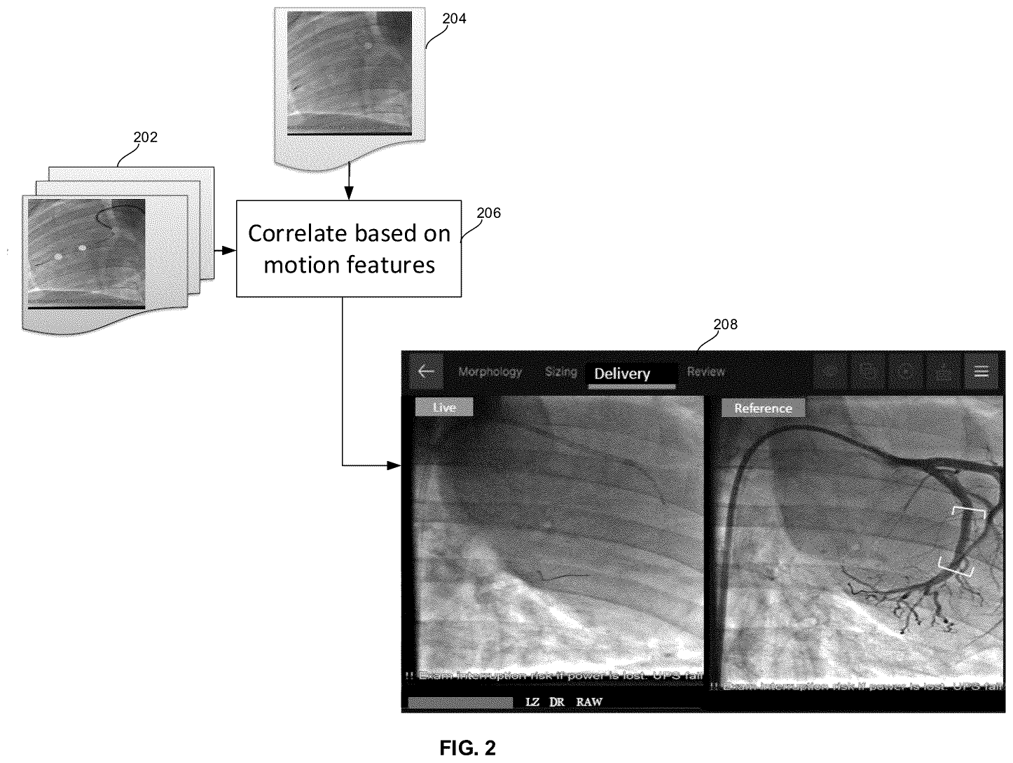

FIG. 2 illustrates an example method of dynamically visualizing the delivery of a device within a blood vessel, which can be used within the system of FIG. 1. Dynamic visualization may be, for example, the correlation, or co-registration, of low dose XAs with one or more high dose XAs. In some examples, dynamic visualization may include the correlation of live low dose XAs, e.g., fluoroscopy images, with a high dose XAs, e.g., cineangiography images. The high and low dose images may be correlated based on motion features detected in, or derivable from, the high and low dose images.

Dynamic visualization may occur in real-time, as the low dose XAs are being captured. The dynamic visualization may allow for a physician to track the position of the device being delivered in the vessel in real time on one or both the low dose XA and a high dose XA. For example, dynamic visualization may allow for a physician to track the position of the device with respect to the vessel tree by providing an indication of the position of the device on the high dose XA, e.g., a vessel map. In some examples, dynamic visualization may allow for a treatment zone identified on a high dose XA to be superimposed on the live low dose XAs such that a physician can track the position of the device relative to the treatment zone.

To dynamically visualize the delivery of a device, first extraluminal images 202 and second extraluminal images 204 may be obtained. The first extraluminal images 202 may be high dose XAs and the second extraluminal images 204 may be low dose XAs. According to some examples, third extraluminal images may be obtained. The third extraluminal images may include one or more XAs obtained prior to the injection of contrast. An XA obtained prior to the injection of contrast may be a pre-contrast image. In some examples, the third extraluminal images may be generated, or captured, at the time the first or second first extraluminal images 202, 204 are generated. For example, a sequence of XA images may be captured prior to contrast being injected. The sequence of XAs may continue to be captured after the contrast is injected. The XAs captured prior to the contrast being injected may be pre-contrast XAs and XAs captured after contrast is injected may be post-contrast XAs. Post-contrast XAs may include low and/or high dose XAs.

The first extraluminal images 202, e.g., high dose XAs, may be used to generate a vessel map. Pre-contrast XAs may be used to identify the working vessel. The second extraluminal images 204, e.g., low dose XAs, may be used to detect and track the device.

The first and second extraluminal images 202, 204 may be correlated based on motion features. The motion features may include, for example, the guide catheter tip, distal endpoint of the working vessel, i-th sample point of the working vessel, optical flow at i-th sample point of the working vessel, translation of the working vessel, optical flow at guide catheter tip, optical flow at distal endpoint of the working vessel, the length of the wire tip, the wire tip at a regional location, the optical flow at the wire tip in the regional location, the proximal endpoint of the wire tip, the distal endpoint of the wire tip, the optical flow at the proximal endpoint of the wire tip, the optical flow at the distal endpoint of the wire tip, or the like. According to some examples, some motion features may be able to be detected in one of or both the first and second extraluminal images 202, 204. For example, the guide catheter tip, distal endpoint of the working vessel, i-th sample point of the working vessel, optical flow at i-th sample point of the working vessel, translation of the working vessel, optical flow at guide catheter tip, and optical flow at distal endpoint of the working vessel may be detected in the first extraluminal images 202. In some examples, the guide catheter tip, distal endpoint of the working vessel, optical flow at guide catheter tip, optical flow at distal endpoint of the working vessel, the length of the wire tip, the wire tip at a regional location, the optical flow at the wire tip in the regional location, the proximal endpoint of the wire tip, the distal endpoint of the wire tip, the optical flow at the proximal endpoint of the wire tip, and the optical flow at the distal endpoint of the wire tip may be detected in the second extraluminal images 204.

The motion features may be used to match the motion phase between the first and second extraluminal images 202, 204. For example, the motion features may include global vessel motion, vessel region of interest motion, and vessel pixel motion. The global vessel motion may be a two-dimensional translation vector which represents the two-dimensional translation at the t-th frame with respect to the first frame. The vessel region of interest motion may be represented by the wire tip region motion. The vessel pixel motion may generally correspond to “n” number of sampled points on the working vessel. Absolute spatial information, e.g., x and y coordinates, and relative spatial information, e.g., optical flow may be determined for the global vessel motion, vessel region of interest motion, and/or vessel pixel motion.

According to some examples, the motion features detected from the first and second extraluminal images 202, 204 may be used to spatially and temporally correlate the motion phase of the first and second extraluminal images 202, 204. The correlation of the first and second extraluminal images 202, 204 may allow for the dynamic visualization of a device within the vessel using a minimum amount of contrast agent. Typically to visualize the location of the device within the vessel, contrast must be injected to be able to identify the contours of the vessel. Accordingly, to consistently view the device during the delivery of the device within the vessel, contrast may have to be continuously injected. Large amounts of contrast may result in negative side effects. Therefore, by correlating a vessel map generated based on the first extraluminal images 202 with the live low dose XA images, e.g., the second extraluminal images 204, the amount of contrast required to track the position of the device during delivery of said device may be minimized.

### Working Vessel Identification

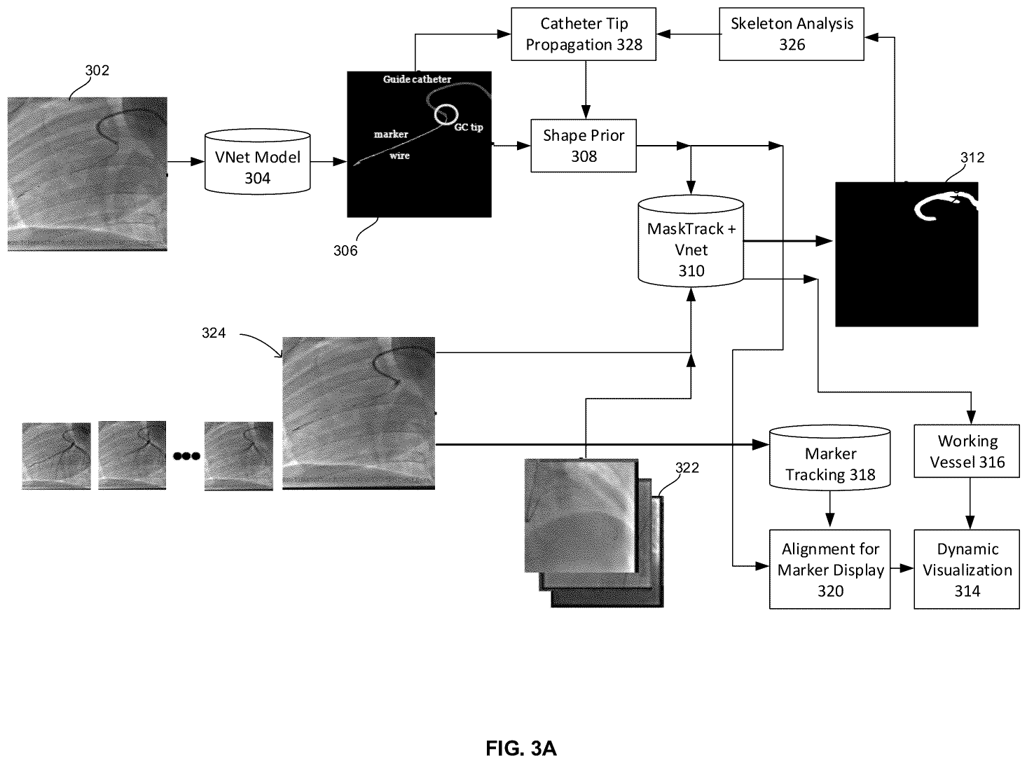

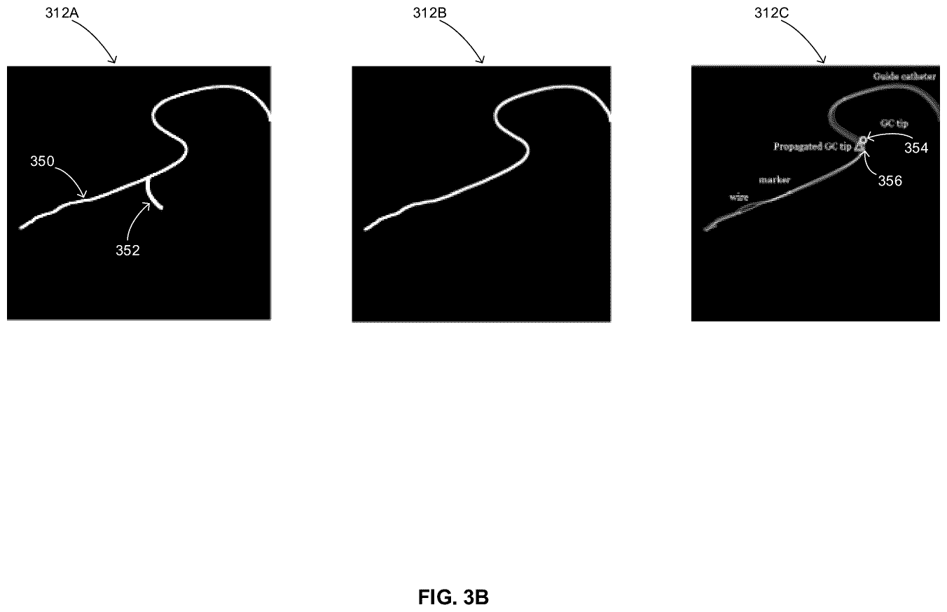

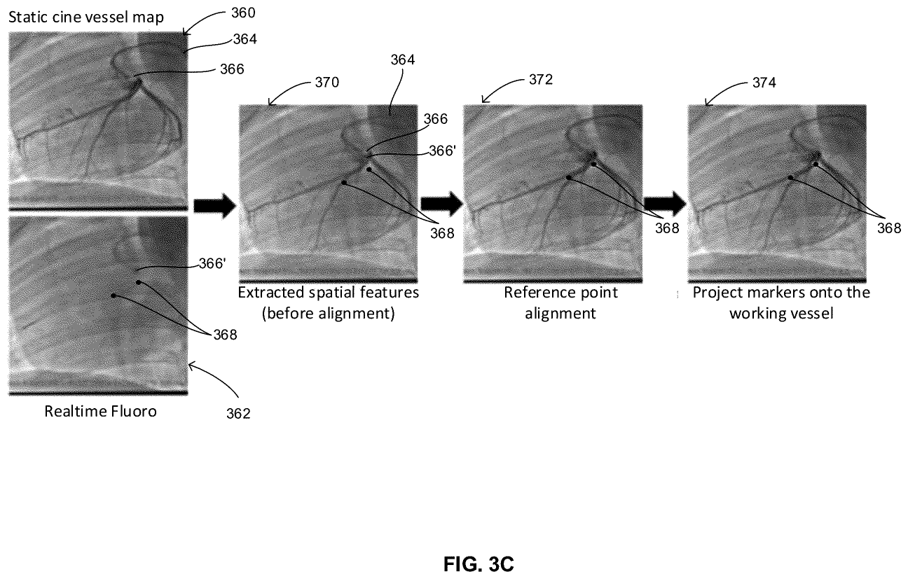

FIG. 3A illustrates an example flow chart for identifying the working vessel and aligning the guide catheter tip for displaying device markers. The following operations do not have to be performed in the precise order described below. Rather, various operations can be handled in a different order or simultaneously, and operations may be added or omitted.

To identify the working vessel, an extraluminal image frame 302 may be received. In some examples, the image frame 302 may be a pre-contrast XA. The pre-contrast image frame 302 may be captured prior to the delivery of the device. For example, the image frame 302 may be captured after the guide wire and/or guide catheter is inserted but before the device is delivered.

The guide catheter and/or guide wire within the image frame 302 may be segmented using AI. The AI may include a deep learning model, such as VNet model 304. The VNet model 304 may be trained to predict a plurality of classes, such as guide catheter, guide wire, device marker, and background. The VNet model 304 may receive, as input, the first image frame 302 and may output multi-label segmentation.

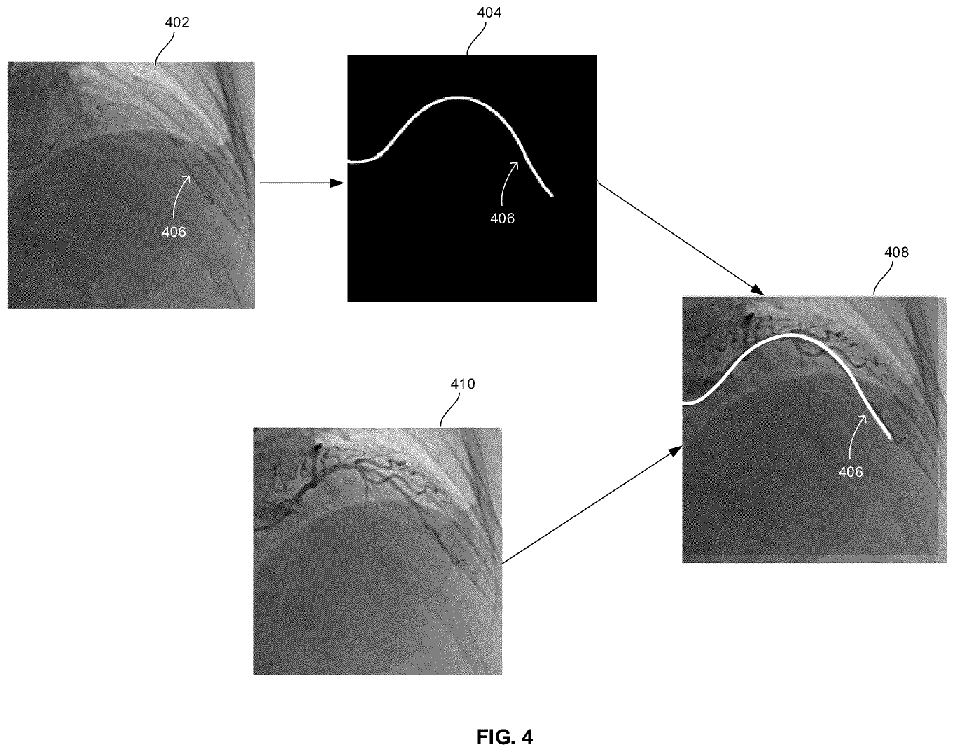

The multi-label segmentation output by the VNet model may be converted into a multi-label mask 306. The multi-label mask 306 may illustrate the working vessel region as a shape prior to the subsequent process. For example, as shown in FIG. 4, the pre-contrast frame 402 may be used to create a multi-label mask or binary mask 404. The binary mask 404 created using the pre-contrast frame 402 may be used as a shape prior for the post-contrast frame. Post-contrast frames may be extraluminal image frames captured after contrast has been injected such that the extraluminal image frames include at least some contrast. According to some examples, after contrast is injected and extraluminal images of the vessel are captured, the contrast may highlight the working vessel as well as the side branches. The contrast may, in some examples, make it more challenging to detect the guide wire in the extraluminal images. Therefore, using the shape prior of the guide wire and/or guide catheter obtained from the pre-contrast frame, e.g., frame 402 in FIG. 4 or frame 302 in FIG. 3A, may allow for the working vessels in post-contrast extraluminal images to be segmented.

As shown in FIG. 4, a pre-contrast XA frame 402 may include a guide wire 406 and/or a guide catheter. The pre-contrast XA frame 402 may be processed such that a binary mask 404 is generated. The binary mask 404 may be of the guide wire 406 in the in the pre-contrast XA frame 402. According to some examples, the binary mask 404 may be used to identify guide wire in the working vessel in the post-contrast frame 410. For example, the binary mask 404 of the guide wire 406 may be overlaid, superimposed, or combined with the post-contrast frame, as shown in frame 408. The shape prior of the guide wire 406, also referred to as the working wire, may be used to segment the working vessel in post-contrast images.

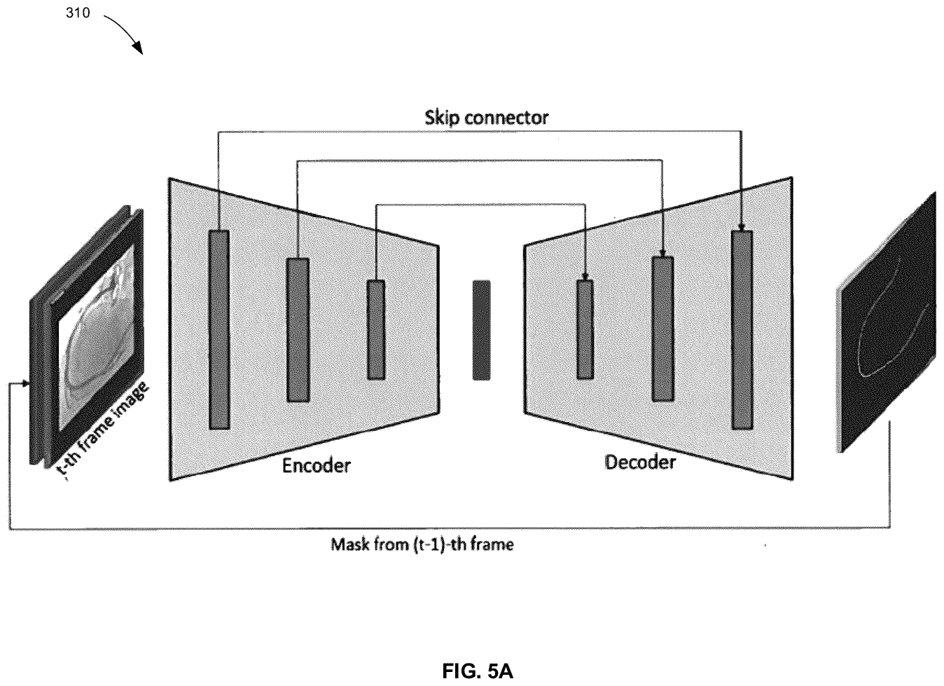

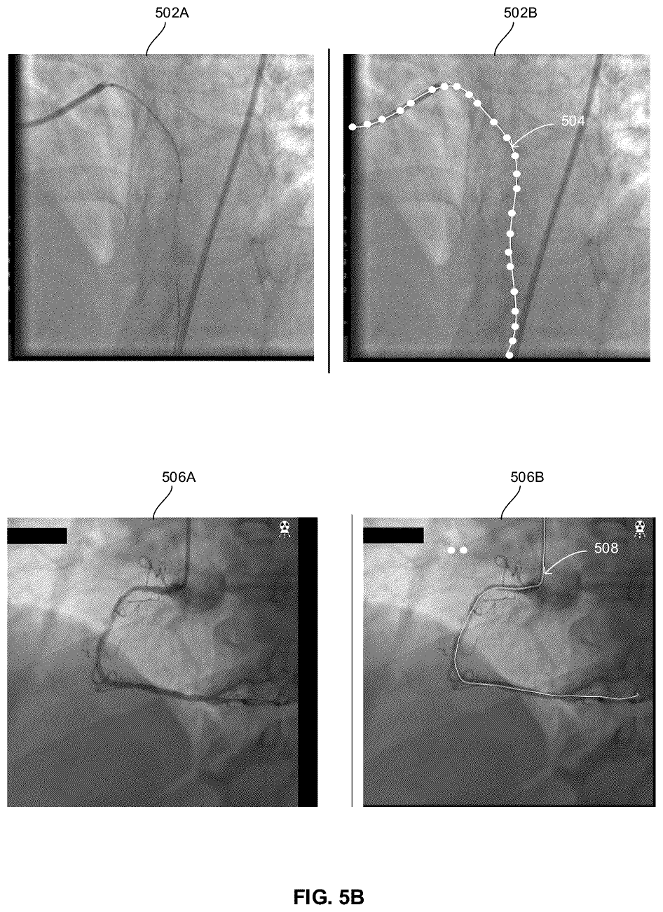

Referring back to FIG. 3A, the shape prior 308 may be provided as input to a deep learning model 310. The deep learning model 310 may be, for example, a MaskTrack+VNet model. The MaskTrack+Vnet model may be trained to identify the working vessel. The MaskTrack+Vnet model 310 may be a spatial temporal model. According to some examples, the MaskTrack+Vnet model 310 may be a spatial-temporal encoder decoder segmentation network, as illustrated in FIG. 5A. The MaskTrack+Vnet model 310 may be trained to predict the working vessel.

The MaskTrack+Vnet model 310 may be trained with a plurality of annotations, as illustrated in FIG. 5B. A pre-contrast XA 502A may be annotated 504, as shown in frame 502B. The annotations 504 for training the MaskTrack+Vnet model 310 may be a set of line strips that follow the trajectory of the guide catheter and guide wire in extraluminal images before contrast is injected. The annotations 504 may, in some examples, correspond to the working vessel trajectory. For extraluminal images captured after contrast is injected, the path following the working vessel line may be labeled. For example, post-contrast XA 506A may be annotated 508, as shown in frame 506B. Annotation 508 may correspond to the work vessel.

When executing the MaskTrack+Vnet model 310, the input into the MaskTrack+Vnet model 310 may be an image frame and prediction information from one or more previous frames. For example, if the current frame is frame “t”, the input into the MaskTrack+Vnet model 310 may be frame “t” and the prediction information from previous frames “t−n”, “t−n+1”, . . . “t−1”, when “n” is greater than 1. According to some examples, the shape prior 308 of the previous frame may be an additional input into the MaskTrack+Vnet model 310. The shape prior propagation may correspond to the “MaskTrack” of the MaskTrack+Vnet model 310. The “VNet” of the MaskTrack+Vnet model 310 is one type of encoder-decoder segmentation network. However, other segmentation networks may be used as part of the shape prior propagation workflow.

Using the shape prior 308 may allow for the MaskTrack+Vnet model 310 may learn, or predict, the anatomical context of the working vessel with respect to the whole vessel tree. According to some examples, the MaskTrack+Vnet model 310, e.g., the spatial-temporal segmentation model, may not be limited by the previous frame. Rather, in some examples, the shape prior 308 may be formulated as previous consecutive frames based on the data, or images, to be processed. The MaskTrack+Vnet model 310 may be executed with a plurality of subsequent frames 324 being used as input in addition to first frame 302. The subsequent frames 324 may be frames captured after the first frame 302 was captured. In some examples, the subsequent frames 324 may include contrast. For example, the subsequent frames 324 may be any combination of high or low dose XA, only low dose XA, or only high dose XA. Based on all the frames, e.g., first frame 302 and the subsequent frames 324, the output of the MaskTrack+Vnet model 310 may be a vessel map 316 of the working vessel.

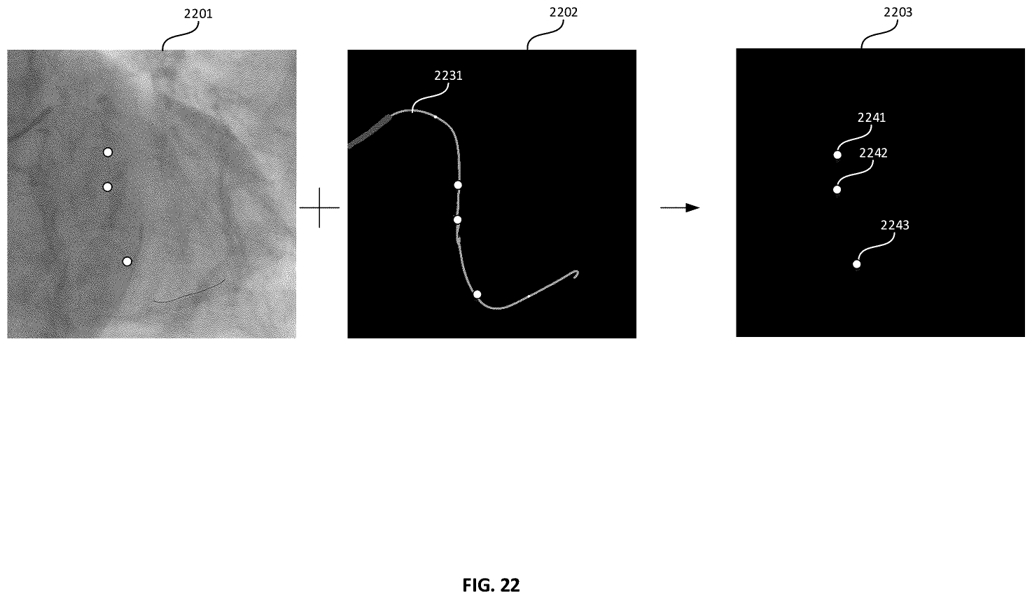

FIG. 22 illustrates a method of determining device marker candidates from the extraluminal image and the shape prior. Device marker candidates may be radio opaque markers captured in the extraluminal image. The device marker candidates may be associated with the markers of an intravascular device or the subject S's anatomy. The marker candidates may be later confirmed to be associated with intravascular devices if certain criteria are met, such as following along the path of the working vessel, moving at a speed in line with the pullback of the intravascular device, within range of the estimated position of the intravascular device, etc. The device marker candidates may be confirmed as device markers by the user or by a trained neural network.

Device marker candidate points, 2241, 2242, and 2243, shown in frame 2203 may be extrapolated from the low-dose XA image 2201. The portion of the working vessel in an extraluminal image, such as pre-contrast high-dose XA frame 2202, may be used to guide the extrapolation of device marker candidate points 2241, 2242 and 2243. In such a situation, the working vessel may serve as a shape prior. To determine the initial shape prior, a pretrained neural network may be used. Various methods, such as rigid-body registration and elastic registration, could be applied to the initial shape prior to alter its shape and increase its diversity. To detect device marker candidates, another neural network may be used. In some examples, the neural network may be a Vnet convolutional neural network. In another example, a MaskTrack+Vnet model, similar to the MaskTrack+Vnet model 310 described in FIG. 3A, may be used to detect device marker candidates. According to aspects of the disclosure, the neural network may be trained based on annotated marker positions of an intraluminal device detected on the extraluminal images. According to aspects of the disclosure, extraluminal images may be high-dose and low-dose XAs. The high dose and low dose XAs may be used for training data in a transfer learning strategy. For the transfer learning strategy, the high-dose and low-dose XA's may be separated.

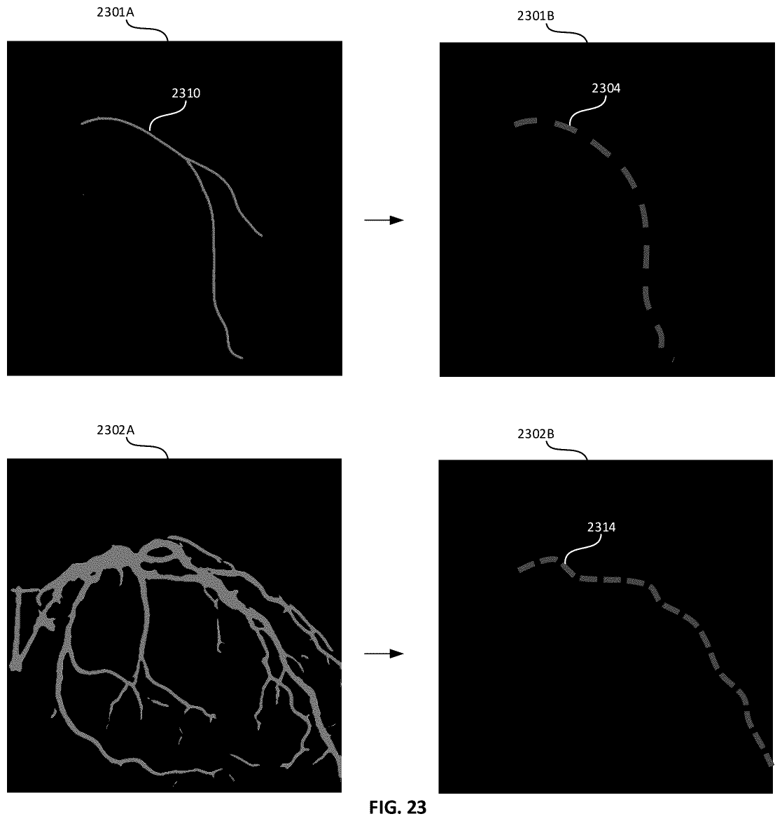

FIG. 23 illustrates two examples to clean up the shape prior. In the first set of frames, frames 2301A and 2301B relate to a direct annotation for a working vessel 2304 from a binary mask frame 2301A. In the second set of frames, frames 2302A and 2302B relate to an inference using a pretrained SVS-Net model for a second working vessel 2314. The illustrated masks may be derived from extraluminal images using the pretrained models as described herein.

The shape prior used by a MaskTrack+Vnet model may require additional annotation. According to some examples, the shape prior for a high-dose extraluminal image may be obtained by directly annotating the device from the same image. In some examples, the shape prior for a low-dose extraluminal image may be obtained by directly annotating the intraluminal device in a pre-contrast, high-dose XA during the same PCI procedure where the low-dose XA was obtained. In other examples where there are no pre-contrast XAs, the shape prior may be directly annotated onto the working vessel in a post-contrast high-dose XA. In some examples, annotation of the working vessel in post-contrast XAs may involve another pretrained neural network, such as an SVS-Net neural network, shown in mask frame 2302A. In the example of FIG. 23, vessel tree mask frame 2302A is extrapolated from a post-contrast, high-dose XA. In some examples, the post-contrast high-dose XA or its vessel tree mask 2302A may be referenced to the low-dose XA containing device markers to refine the mask for the working vessel, which will be used as the working vessel in the shape prior.

The trained model may be applied to low-dose XAs from the treatment stage of a PCI to detect candidate markers. In some examples, the probability map output from the neural network model may be thresholded, and candidate marker positions may be extracted by finding center points of each connected component in the thresholded image.

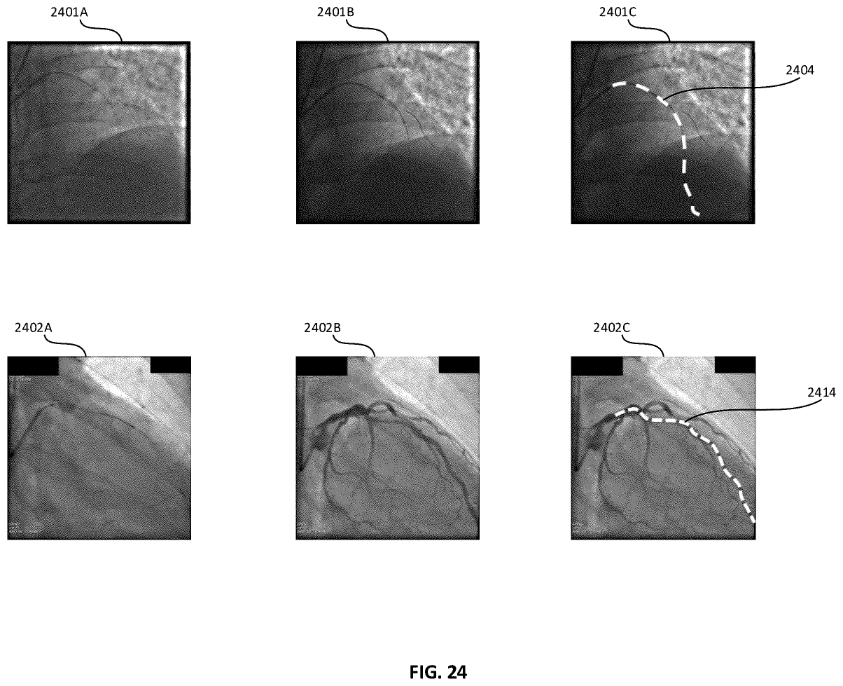

FIG. 24 illustrates the annotation of the shape prior for low-dose XA training data acquired from PCI in different conditions. Frames 2401A, 2401B, and 2401C illustrate an example with pre-contrast high-dose XA. Frames 2402A, 2402B, and 2402C illustrate an example with only post-contrast high-dose XA. Frames 2401A and 2402A depict low dose XA images. Frames 2401B and 2402B depict high-dose XA images. Frames 2401C and 2402C depict the annotated working vessel 2404 and 2414, respectively, depicted on the high dose frames. The annotated working vessels 2404 and 2414 are to be used as the shape prior.

Referring back to FIG. 3, the guide catheter and wire tip may be segmented from the subsequent frames 324. According to some examples, the guide catheter and wire tip may be segmented from the subsequent frames using the MaskTrack+Vnet model 310. In a typical percutaneous coronary intervention (PCI) workflow, the subsequent images frames 324 are not recorded. The number of recorded subsequent frames 324, such as low dose XAs, is typically less than the number of high-dose XAs taken during the diagnostic phase. To address the reduced number of low dose XAs typically captured, the system may augment the high dose XAs to generate augmented low dose XAs 322 such that the augmented low dose XAs 322 can be used as input into the training process for the MaskTrack+Vnet model 310 in the example of guide catheter and wire tip segmentation.

According to some examples, at least two factors may be applied to the contrast and signal-noise-ration (“SNR”) to transform high dose XAs into augmented low dose XAs 322 that can be used to train the MaskTrack+Vnet model 310, or a similar model, to segment the guide catheter and wire tip. The high dose XAs captured before delivery of a device or during a PCI workflow, may be processed to extract the neighborhood of target objects, such as the guide catheter, wire tip, or the like. In some examples, the high dose XAs may be processed using dilation to extract the neighborhood of target objects. The intensity contrast of the target objects in the high dose XAs may be determined. In some examples, a signal-noise-ration (“SNR”) may be determined based on the high dose XA and low dose subsequent frames 324. According to some examples, the augmented low dose XAs 322 may be generated using generative adversarial network (“GAN”) type models, or other generative type models.

The MaskTrack+Vnet model 310 may be trained using the augmented low dose XAs 322 as well as captured low dose XAs, such as subsequent frames 324, as input. The MaskTrack+Vnet model 310 may be trained to automatically detect the guide catheter and wire tip. The MaskTrack+Vnet model 310 may be trained to predict a segmentation of the guide catheter and wire tip. For example, when executed, the MaskTrack+Vnet model 310 may receive subsequent frames 324 and provide frame 312 as output, where frame 312 may be the segmented guide catheter and/or the wire tip.

In some examples, frame 312 may be a skeleton frame. The skeleton frame 312 may be analyzed 326 for reference point propagation. For example, as shown in FIG. 3B, skeleton frame 312A may contain one or more side branches 352, other than the working vessel 350. According to some examples, to keep the shape prior substantially similar to the working vessel 350, as illustrated in frame 312B, skeleton analysis 326 may be performed. Skeleton analysis 326 may include, for example, skeleton path search, side path removal, etc. In some examples, the output from MaskTrack+Vnet 310 is a working vessel map that is obtained after injecting contrast into the vessel. The guide catheter tip may be invisible due to the occlusion by contrast X-ray attenuation. The shape prior from frame 306 showing the multi-labels can be aligned with the skeleton analysis 326 result using a geometric transformation. After the alignment, as illustrated in frame 312C in FIG. 3B, the guide catheter tip in frame 306 (indicated by the circle 354) may be projected to the nearest neighboring skeleton point as the propagated guide catheter tip (indicated by the triangle 356) in the contrast injected XA.

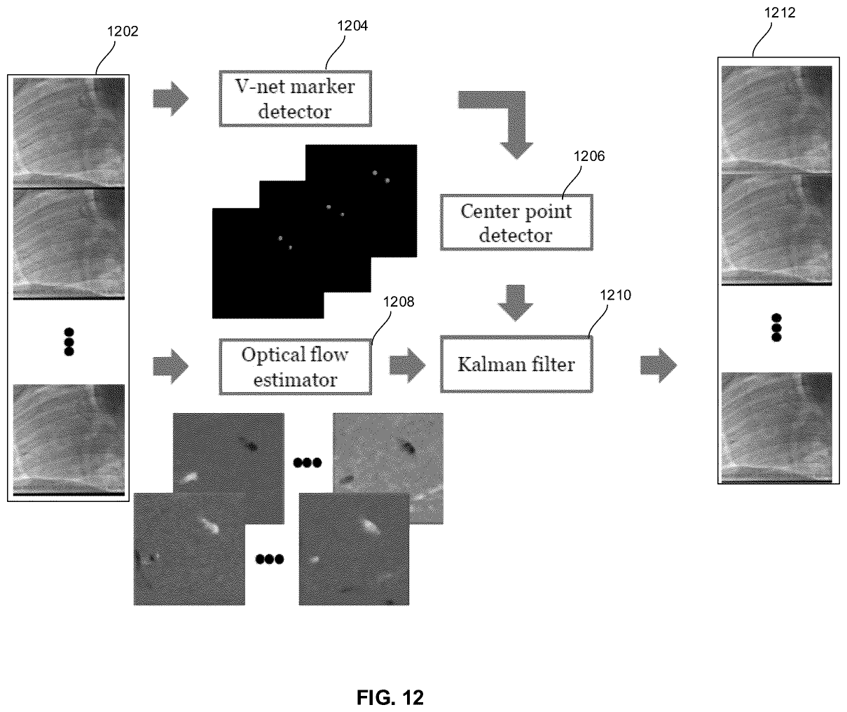

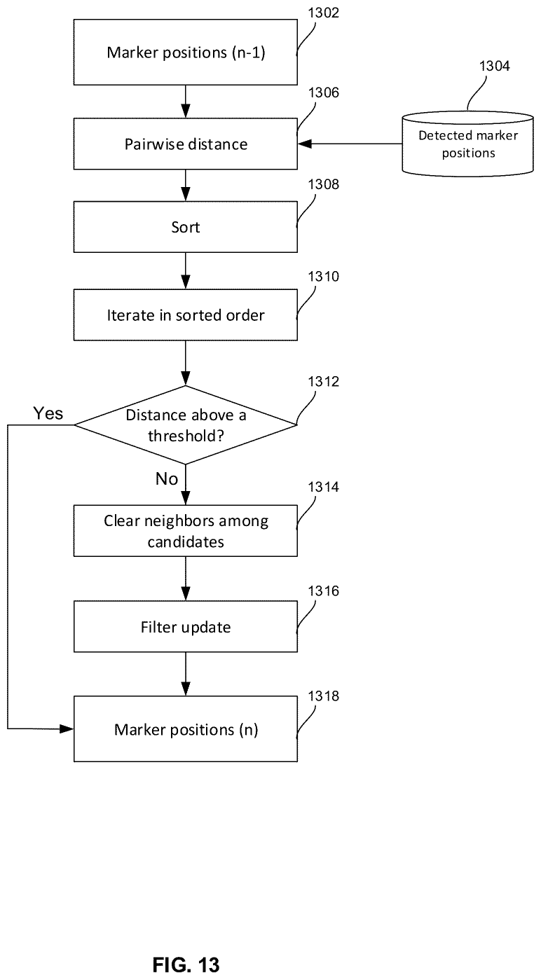

According to some examples, the frames 324 may be used to detect and track 318 the position of the marker on the device being delivered within the vessel. The markers on the device may be detected and tracked 318 using a AI model, a convolution neural network, or the like. Details pertaining to how the marker is detected and tracked is discussed in more detail with respect to FIGS. 12 and 13.

The working vessel map 316 along with the guide catheter tip location may be obtained using MaskTrack+Vnet 310 for the high dose XA, as illustrated in frame 360 in FIG. 3C. For example, the guide catheter tip location 366 may be automatically identified by MaskTrack+Vnet 310 along where a curve going through the main vessel 364. With a similar approach, the guide catheter and its tip location 366′ can be identified for the live low dose XAs, as illustrated in frame 362 in FIG. 3C. The device markers 368 in the live XAs, e.g., frame 362, may be detected and tracked. In some examples, the imaging posture between vessel map and live low dose XAs may be slightly different. In such an example, the image context between them may not be aligned, as illustrated in frame 370. To address this issue, the guide catheter tip 366, 366′ in both the vessel map 360 and the live XAs 362 can be aligned by translating one image frame to another, so that the image context can be properly fused together, as illustrated in frame 372. The markers 368 can then be displayed on the working vessel using nearest neighboring points or other geometric projection for marker display 320, as illustrated in frame 374.

In some examples, the region of interest may be determined using detected device marker candidates along a working vessel. A candidate region of interest (cROI) may denote a box that contains one or more device marker candidate's center points as detected by the neural network model. According to some examples, for each device marker candidate detected within a frame of low-dose XA, a cROI box may be determined with its center point set to the detected device marker candidate's center point. To reduce the number of cROI boxes without losing track of the device markers, various methods, such as a non-maximum suppression step (NMS) may be applied. In another example, the cROI boxes may be determined from the working vessel. In some examples, the working vessel may be determined in a pre-contrast, high-dose XA and overlaid on live feed of the low-dose XA.

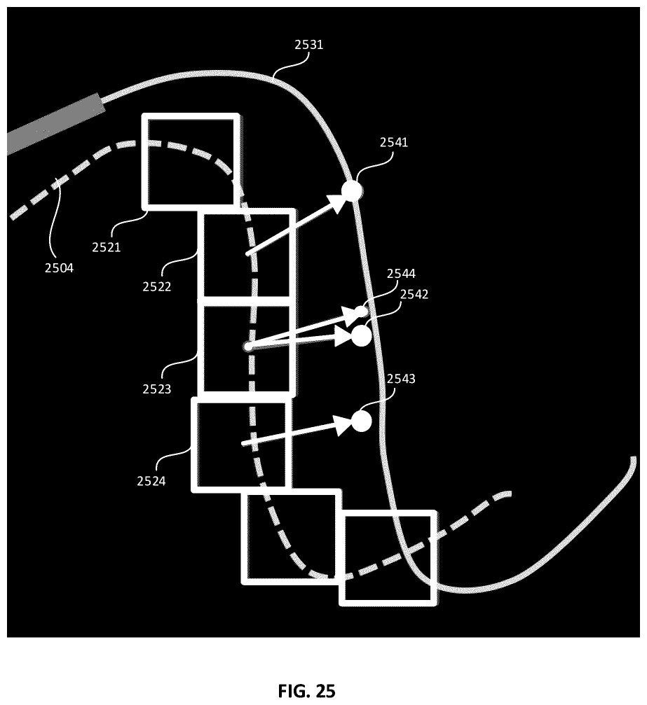

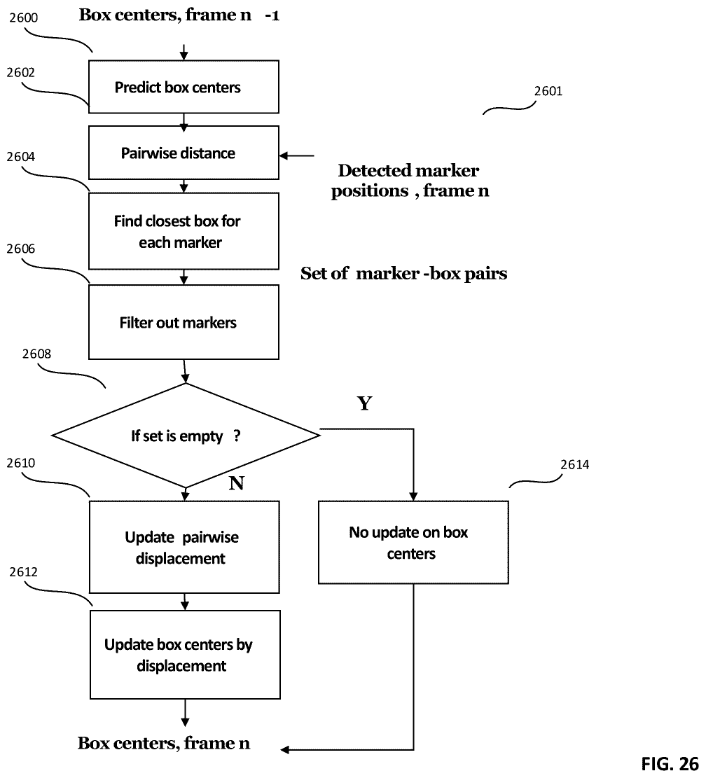

FIG. 25 illustrates a method of determining regions of interest based on detected device marker candidates. The method may consist of matching detected device marker candidates with the nearest cROI marked along the working vessel. This method of pairing the detected device marker candidates with cROI acts as a confirmatory step to ensure the device markers that are tracked fall along the working vessel. In FIG. 25, the working vessel 2504 defined by a shape prior may be represented by a number of spatially contiguous boxes, including boxes 2521, 2522, 2523 and 2524. The boxes may be the same size. In some examples, the boxes may be varying sizes to fit along the length of the working vessel 2504. In some examples, the size and number of boxes may be input by a user or automatically selected by the AI algorithm. The boxes may be associated with device marker candidates, such as 2541, 2542, and 2543, detected along the path 2531 of the intravascular device. The device marker candidates may be associated with nearest of the number of contiguous boxes. For example, device marker candidates, 2541, 2542, 2543 may be associated with boxes 2522, 2523, and 2524, respectively. In some examples, boxes such as box 2521 may be kept as the device is constantly moving along the path 2531 over time of the treatment phase. At a different time of the treatment phase, the device marker may fall into different regions of interest. The method may keep a constant number of regions of interest and update them according to each frame of the low dose XA image, as described below in FIG. 26. Alternatively, in some examples, boxes without device marker candidates detected within them may not be qualified to be regions of interest, such as box 2521.

The device marker candidate points may be the center of their associated cROIs. Initial working vessel trace points are determined from the high dose XA image's working vessel detection 2504. Given the working vessel detection 2504 from the high dose XA image used as the shape prior, a breadth first search (BFS) algorithm may be used to sort trace points along its path. Associated cROIs with minimum overlap, nearest to each detected marker in a new frame of low dose XA image and along this working vessel trace may be selected. In some examples, the device marker candidate may not align with the center of the nearest box. In this case the AI model may estimate a closest center point to the device marker candidate along the path of the vessel as the center of the box. For example, device marker candidate 2542 is nearest to box 2523. Because device marker candidate does not fall at the center of box 2523, the model may create center point 2544 nearest to the device marker candidate 2542. The Kalman filter in FIG. 26 addresses the issue that device marker candidates does not align with the center of the nearest box. In the end of the algorithm of block 2612, the centers of the regions of interest are close to the device marker candidates. In some examples, a cROI that contains device markers detected from the neural network model may be determined as a region of interest. In another example, a cROI box that contains a centroid within a predetermined distance to the device markers detected from the neural network model may be determined as a region of interest. The device markers may fall outside the box as long as the device marker is within a given distance to the box centroid.

In another example, a cROI or box may be determined as a region of interest, if it passes a persistency criterion. In some examples, a persistency criterion may be a check for the presence of a region of interest for more than 5 frames of low-dose XAs among the past and present 9 consecutive frames. In some examples, a cROI may be determined as a region of interest if it is distal to the guide catheter tip. In the situation where contrast is injected during the treatment phase, the pixel value distributions within a given region of interest may be compared with those from the same region of interest in past frames. In some examples, frames with contrast injection may be detected across the low-dose XA series.

One major limitation of prior methods is the need of a device-specific template to automate device marker detection and tracking. For advanced treatment techniques, such as kissing balloon, that require delivery and coordination of multiple devices, each device may need to be registered separately before use, complicating the procedure's overall workflow. This fully automated method improves real-time visualization of device markers in low-dose XAs, by detecting regions of interest containing device marker candidates within each frame of the live feed of low-dose Xas. This method is fast and robust to variation in the treatment workflow, thereby expanding the usability of device marker dynamic visualization to help physicians in complex clinical situations. This method may also be extended to other applications requiring device marker visualization, such as electrophysiology (EP), pulmonary and peripheral vascular angiography applications, or involving other imaging modalities such as MRI or IVUS in image guidance.

FIG. 26 illustrates a flow diagram of an example workflow of tracking regions of interest in two subsequent frames of low-dose Xas. According to some examples, each region of interest may be represented by the corresponding box center points as depicted in FIG. 25. The boxes may have a fixed width parameter and a fixed height parameter. The following operations do not have to be performed in the precise order described below. Rather, various operations can be handled in a different order or simultaneously, and operations may be added or omitted.

In block 2600, a model may estimate box center points of candidate regions of interest, based on extraluminal images. In some examples, a Kalman filter may be used to estimate these box center points. According to some examples, to reduce false positives, after the prediction step of each Kalman filter iteration, the predicted marker position may be compared to the detected marker positions 2601. In the prediction step, according to some examples, a loss function between pixel values within each box and within an adjacent box of the same size, from two subsequent frames of low dose XA images may be computed. In some examples, the loss function may be a 2-D cross correlation. In another example, the loss may be some other similarity or dissimilarity criteria used for image registration. The displacement between these two boxes with the minimized loss function, either the dissimilarity criterion or the negative of the similarity criterion, may be used to predict the motion of the box. According to another example, the displacement may be computed using an optical flow to predict the motion of the box. In some examples, the Lucas-Kanade (LK) method may be used to compute the optical flow.

In block 2602, the model may determine pairwise distances between the predicted box center points and detected device marker center points in the current frame. The model detects device marker candidates' positions on the extraluminal images.

In block 2604, the model may identify and pair each newly detected device marker center point to its nearest box. Among all the pairs, the corresponding distance may then be compared with the box width and height to determine how close a device marker is relative to the paired nearest box.

In block 2606, the model may filter out pairs with device markers outside, or with a distance larger than a predetermined threshold to its nearest box, or pairs with device markers inside the guide catheter. In the step, the model reduces the number of false positives detected by the model by removing any markers that are far from the boxes. The false positives may be a result of foreign matter or the anatomy of subject S.

In block 2608, a determination is made regarding whether the pairs with markers close to the boxes require updating or adjustments on the box center points. In some examples the detected markers may not be the natural center point of the nearest box. In some examples, multiple markers detected within a box, wherein only one marker can be the natural center point of the nearest box. This is allowed as the method may only average all displacements into one constant displacement, see block 2610 of FIG. 26, to translate all trace points, in some instances, by the same amount. Therefore, a one-to-one correspondence is not required. If no pairs exist after filtering, or no pairs need to be recentered, the model may move to block 2614. If pairs require adjustments to update the boxes center point, the model may proceed to block 2610.

In block 2614, no pairs remain to be adjusted, no position update will be made to the boxes in the current frame. In block 2610, if adjustments need to be made to the box center points, an update step of the Kalman filter may be performed. In the update step, each detected marker's position may be used to update the Kalman prediction 2600 of its nearest box center position, and displacements between each markers' nearest box center points before and after the Kalman update may be averaged across all remaining pairs.

In block 2612, the model may update the box center points based on the determined average displacement.

Referring to FIG. 3, working vessel 316 may be provided for output as a vessel map when dynamically visualizing 314 the delivery of the device in the vessel. The detected and tracked markers 318, once aligned 320, may be provided for output. For example, the markers may be provided for output on the vessel map to provide an indication of the location of the device in real time, without having to inject additional contrast. According to some examples, a treatment zone, initially identified on the vessel map, may be provided for display on the live low dose XA image such that the location of the device on the low dose XA can be seen relative to the treatment zone. In some examples, the marker may be provided for output on the vessel map to provide an indication of the location of the device in real time with respect to the treatment zone.

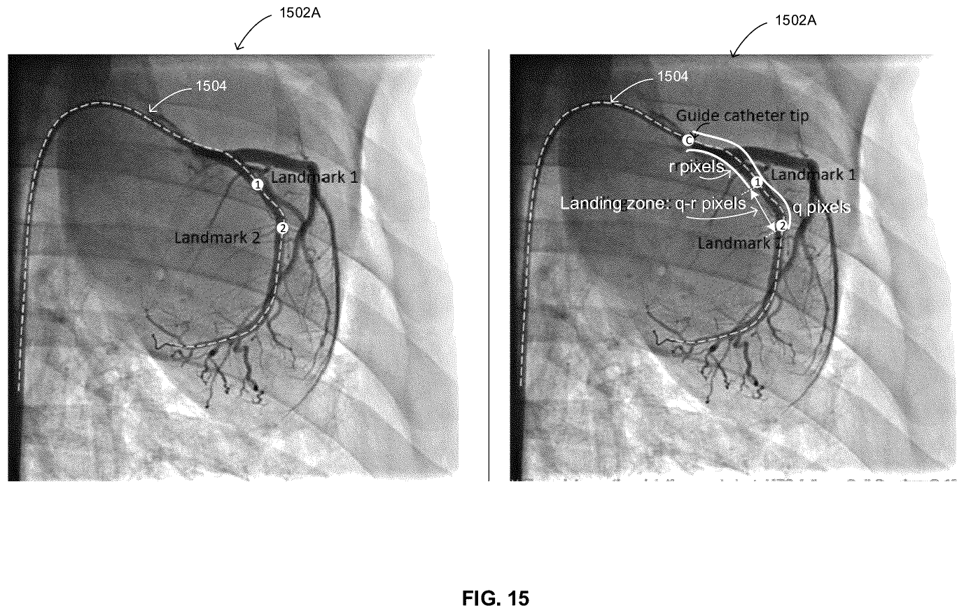

Referring to FIG. 15, one or more landmarks along the working vessel may be identified. For example, the working vessel 1504 may be identified in frame 1502A. Two landmarks, landmark 1 and landmark 2, may be identified. The landmarks 1, 2 may be automatically identified and/or identified based on a received input. For example, the system may receive an input via the computing device, touch screen, etc. corresponding to the selection of landmarks. The landmarks 1, 2 may correspond to a working zone or a device landing zone. The landing zone may correspond to coordinates relative to the catheter tip “C”. In some examples, the distance between the landmarks 1, 2 and the catheter tip “C” may be determined in each high dose XA. For example, the distance between the catheter tip C and landmark 1 may be “r pixels” and the distance between catheter tip C and landmark 2 may be “q pixels.” Pixels along the working vessel trajectory within distance range, e.g., the distance between the landmarks, may correspond to the determined landing zone. As illustrated in FIG. 15, the pixels in the landing zone may be determined as “q pixels” minus “r pixels.” The landing zone may, in some examples, be referred to as a treatment zone. According to some examples, the vessels may be invisible in low dose XAs. In such an example, when the guide catheter tip location “C” is available, combining the above pixel distance information may provide a treatment zone on the output. Providing a treatment zone on the output, e.g., on the display, may assist a user in the delivery of a device.

### Multi-Scale Motion Features

XAs taken during PCI typically present mixed motion of a patient's heartbeat and breath. The motion makes it challenging to correlate live XA images with a previously captured XA image. Further, there is typically an inherent projection property that the vessel shown in the high and low dose XAs is a two-dimensional projected geometry, rather than a three-dimensional geometry. Due to such, accurately identifying physical pixel correspondences of the high and low dose XAs may be challenging and impractical. However, different pixels may present different motion patterns and the motion of different pixels can show similar patterns. The multi-scale motions features may, therefore, be used to correlate the high and low dose XAs. By using multi-scale motion features, the process for correlating high and low dose XAs may be completed without using external sensors, such as an EKF, time stamps, or time and computationally intensive methods.

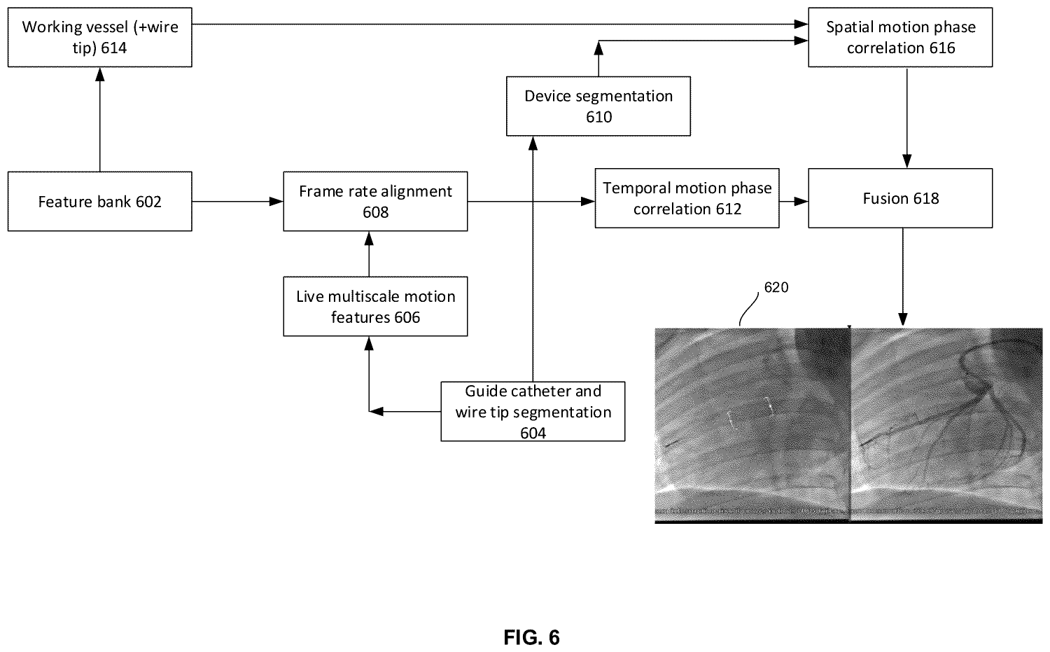

FIG. 6 illustrates an example method of correlating live XA images, such as low dose XAs or fluoroscopic images, with a previously captured XA, such as a high-dose XA, based on motion features. The following operations do not have to be performed in the precise order described below. Rather, various operations can be handled in a different order or simultaneously, and operations may be added or omitted.

To correlate the motion phase of a high dose XA and low dose XAs, image features may be identified that correspond to the mixed motion. A multiscale motion feature bank 602 may be generated. The multiscale motion feature bank may provide motion information from various perspectives and may be used for the subsequent phase matching process. The multiscale motion features may include global vessel motion, vessel region of interest motion, vessel pixel motion, or the like.

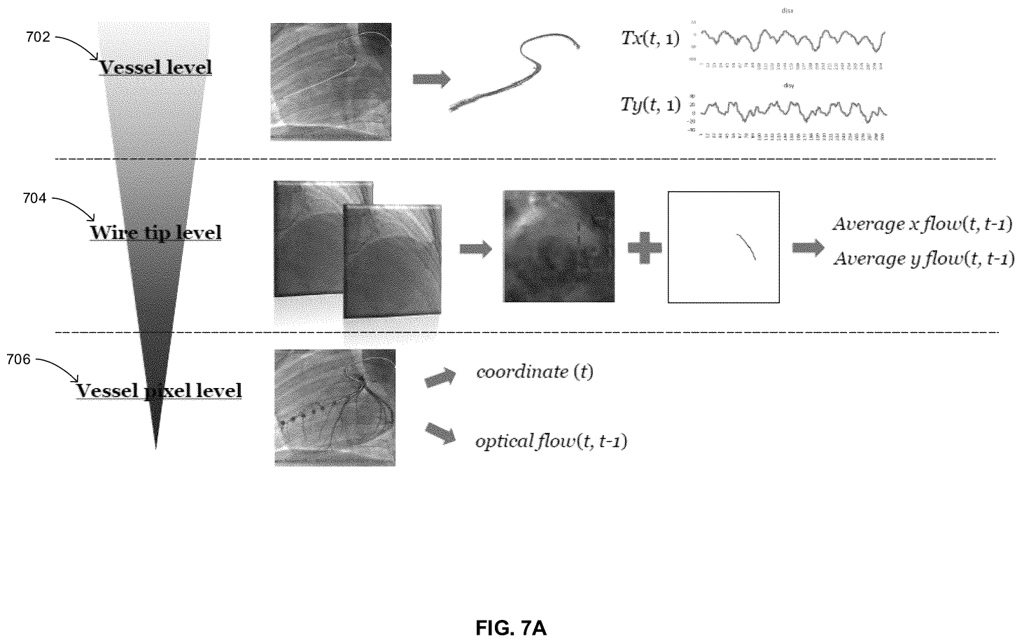

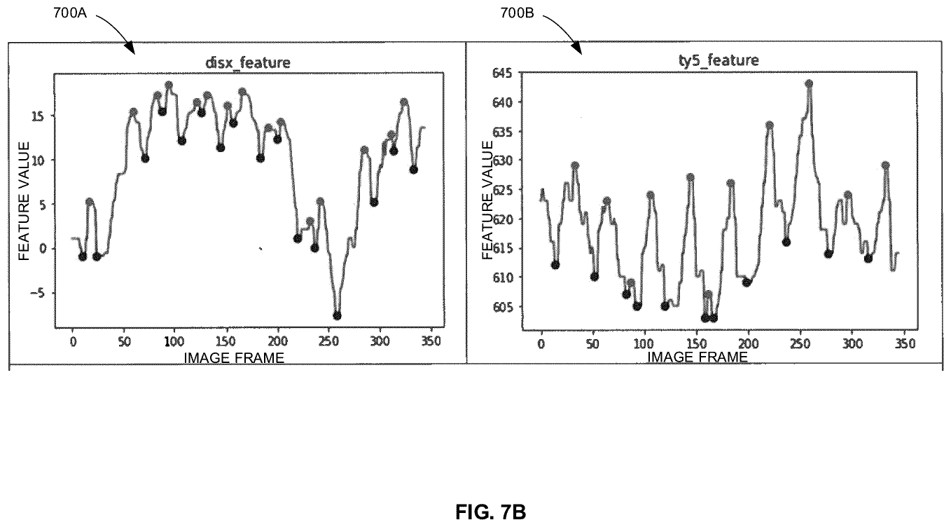

FIG. 7 illustrates global vessel motion 702, vessel region of interest motion 702, and vessel pixel motion 706. The vessel level motion 702 may correspond to a two-dimensional translation vector. The two dimensional translation vector may be represented as: Tx(t,1) and Ty(t,1). The two-dimensional translation vector may be the two-dimensional translation at the t-th frame with respect to the first frame.

The vessel region of interest motion 704 may correspond to the wire tip level motion. The wire tip level motion may include, for example, absolute spatial information and relative spatial information. The absolute spatial information may correspond to coordinates, such as x and y coordinate. Relative spatial information may correspond to the optical flow between two adjacent frames.

The vessel pixel motion 706 may correspond to “n” number of sampled points along the working vessel. According to some examples, the sampled points may be between the guide catheter tip to the distal endpoint of the vessel. The sample points may be identified, or detected, using the VNet model 304 and/or the MaskTrack+VNet model 310. According to some examples, absolute spatial information and relative spatial information may be determined for the vessel level pixel motion.

Referring back to FIG. 6, the segmented guide catheter and wire tip from the low dose XAs obtained during PCI may be used to derive multiscale motion features. For example, the segmented wire tip may be used to label wire tip level motion features 704. In some examples, the working vessel may be sampled between the wire tip and the guide catheter tip to detect pixel level features 704. For example, for each pixel, the motion feature type may be derived in period form. The pixel level 704 motion features may be derived, or determined, using the high or low dose XAs. The type of motion features detected may be based on whether the features are detected from a high or low dose XA.

The motion features may include, for example, the guide catheter tip, distal endpoint of the working vessel, i-th sample point of the working vessel, optical flow at i-th sample point of the working vessel, translation of the working vessel, optical flow at guide catheter tip, optical flow at distal endpoint of the working vessel, the length of the wire tip, the wire tip at a regional location, the optical flow at the wire tip in the regional location, the proximal endpoint of the wire tip, the distal endpoint of the wire tip, the optical flow at the proximal endpoint of the wire tip, the optical flow at the distal endpoint of the wire tip, or the like. According to some examples, some motion features may be able to be detected in one of or both the high dose and low dose XAs. For example, the guide catheter tip, distal endpoint of the working vessel, i-th sample point of the working vessel, optical flow at i-th sample point of the working vessel, translation of the working vessel, optical flow at guide catheter tip, and optical flow at distal endpoint of the working vessel may be detected in the high dose XAs. In some examples, the guide catheter tip, distal endpoint of the working vessel, optical flow at guide catheter tip, optical flow at distal endpoint of the working vessel, the length of the wire tip, the wire tip at a regional location, the optical flow at the wire tip in the regional location, the proximal endpoint of the wire tip, the distal endpoint of the wire tip, the optical flow at the proximal endpoint of the wire tip, and the optical flow at the distal endpoint of the wire tip may be detected in low dose XAs.

The motion features may form a periodic pattern when graphed. For example, as shown in FIG. 7B, graph 700A is a representation of the x translation of global vessel motion and graph 700B is a representation of the vessel pixel motion y translation of a sample point along the working vessel. The horizontal axis for each graph corresponds to the frame number and the vertical axis corresponds to the feature value. As illustrated, the motions features in graphs 700A, 700B have a substantially periodic pattern.

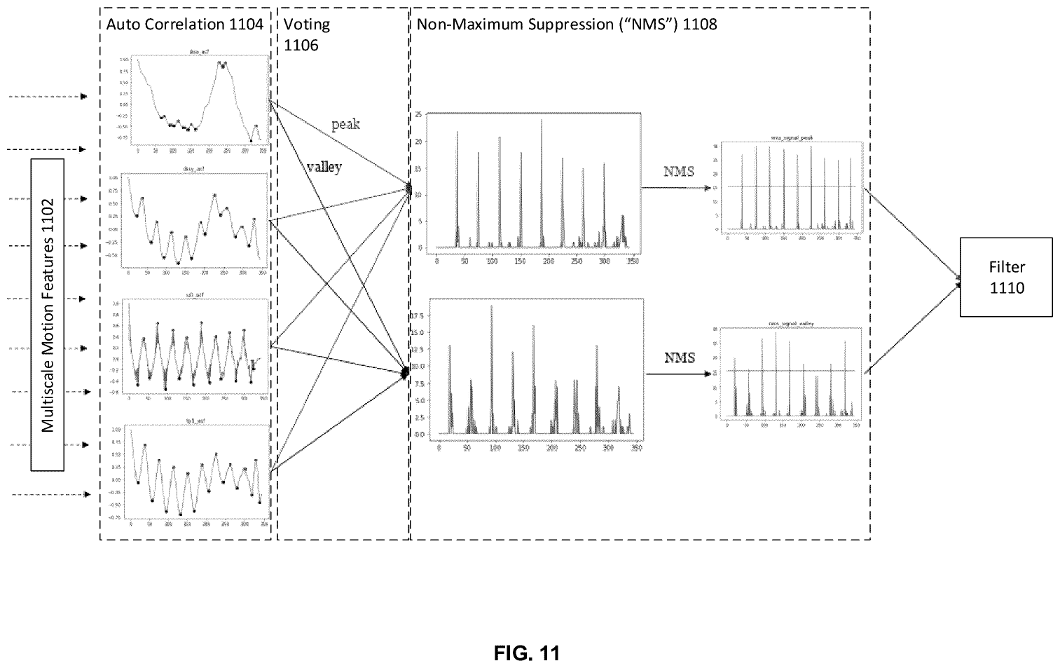

As illustrated in FIG. 11, a heartbeat period of a patient may be determined based on the motion features. An auto-correlation function (“ACF”) may be applied to the multiscale motion features 1102. For example, the motion features 1102 may be raw motion features that are converted to ACFs. The autocorrelated motion features may, in some examples, be graphed such that peaks and valleys are identified. For example, the signals associated with the motion features may be processed, such as by finding the derivative of the signals. According to some examples, the peaks and valleys of the motion features may be identified using the first or second order derivative of the signals. The distance between adjacent peaks may be determined. In some examples, the distance between adjacent valleys may be determined. The distances between peaks and valleys may be voted 1106 in binned spaces. According to some examples, the motion features can present certain noises in the temporal axis. For example, peak #1 in feature #1 may be at the 10^{th} frame, while peak #1 in feature #2 may be at the 11^{th} frame. However, the features are likely point to correlated and/or associated with the same heartbeat moment. As a result, the temporal space can be binned into a coarser resolution. For example, the 10^{th} and 11^{th} frames can be merged as one bin, and two votes could be placed into this bin. By such, the votes for a heartbeat moment can be concentrated and enhanced.

After voting 1106, a NMS 1108 may be applied. The NMS 1108 may refine the heartbeat period in both the peak and valley domains. In some examples, the voting results may still present multiple peaks around a heartbeat moment, depending on the binning size. The NMS may be performed to suppress noisy votes and preserve the maximal vote in a local temporal neighborhood. As a result, the heartbeat moment and cycle can be extracted more reliably.

A filter 1110, such as a median filter or other statistical filter, may be used to identify the heartbeat period. The heartbeat period may be used when correlating the motion phases of the low and high dose XAs.

### Spatial-Temporal Motion Phase Matching

Referring back to FIG. 6, the live multiscale motion features may be used to temporally correlate the motion phases of the high and low dose XAs. The motion feature bank 602, live multiscale motion features 606, and frame rate alignment 608 may be used to temporally correlate the high and low dose XAs 612. Temporally correlating the high and low dose XAs may include, for example, identifying a corresponding high dose XA for each low dose XA. The corresponding high dose XA may have substantially similar temporal consistency as the low dose XA. According to some examples, the high dose XAs may be generated, or captured prior to the delivery of the device. The temporal consistency may be based on, for example, when during the heart cycle the low and high dose XAs were captured. According to some examples, temporal consistency in the low and high dose XAs may be determined by comparing the cardiac motion in the corresponding images. Cardiac motion may, over time, repeat itself such that the cardiac motion observed in the low dose XA sequence may appear similar to the cardiac motion observed in the high dose XA sequence. The similarity between the cardiac motion in the low and high dose XA sequence may be determined, or quantified, using representative motion features. For example, the temporal evolutions of the representative motion features may be correlated and used to determine the cardiac motion. For example, cardiac motion in the low and high dose XAs, in some examples, may be determined based on the position of a guide wire tip or the position of the guide catheter tip. In another example, cardiac motion may be determined based on the displacement of the guide wire tip or guide catheter tip over one sampling time interval of the angiography imaging system, or by any other physical mechanism of the like, or their combinations. In some examples, if the frame rates during acquisition of high and low dose XA are different, the motion features may be used to align the frame rate 608.

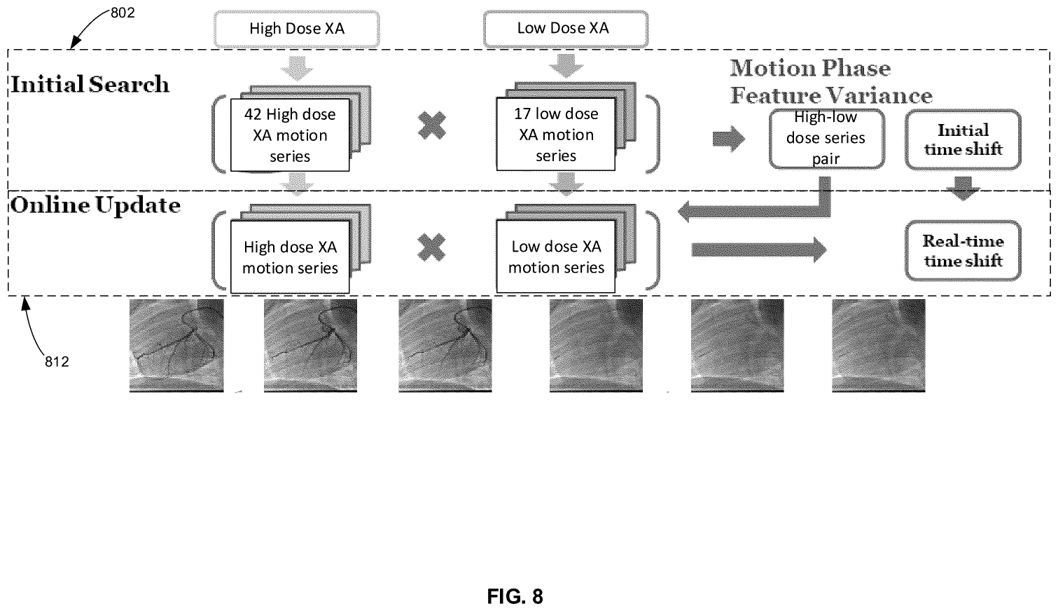

FIG. 8 illustrates an example method for temporally correlating the motion phases of the high and low dose XAs. To temporally correlate the high and low dose XAs, an initial search is performed. In some examples, after the initial search 802 is performed, an online update 812 is performed. According to some examples, the initial search 802 may identify correlation results that are used for subsequent online updates 812.

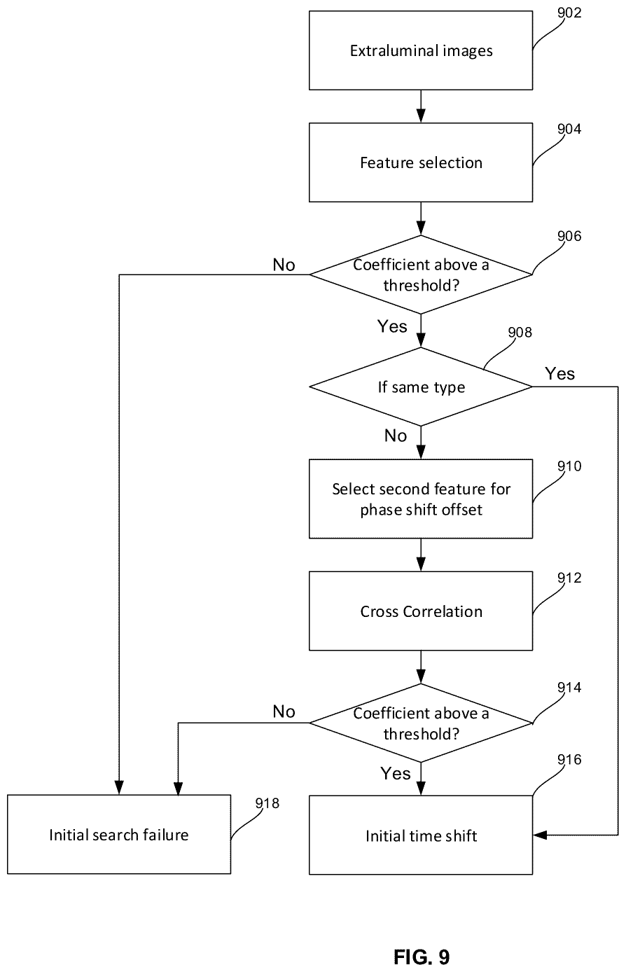

FIG. 9 illustrates an example method for performing the initial search. The initial search may be performed to determine the initial time shift. The following operations do not have to be performed in the precise order described below. Rather, various operations can be handled in a different order or simultaneously, and operations may be added or omitted.

In block 902, extraluminal images, such as low and high dose XAs, are obtained. Motion features may be derived from the high and low dose XAs, respectively.

In block 904, the motion features may be selected from the high and low dose XAs. According to some examples, multiple motion features may be selected from both the high and low dose XAs. In some examples, the motion features may be resampled at a common frame rate. The frame rate may, in some examples, be used to estimate a time interval between incoming low dose XAs during the subsequent online update 812.

A correlation coefficient may be determined for the motion features in the high and low dose XAs. The correlation coefficient may be determined using a normalized cross correlation (“NCC”). The NCC may be determined, for example, using the following equation:

$$r ( X , Y ) = \frac{{\sum }_{k} ( {X}_{k} - \overline{X} ) ( {Y}_{k} - \overline{Y} )}{\sqrt{{\sum }_{k} {( {X}_{k} - \overline{X} )}^{2} {\sum }_{k} {( {Y}_{k} - \overline{Y} )}^{2}}}$$

In the above equation, X and Y are the high-dose and low-dose XA motion features, Xₖ and Yₖ are the k-th samples of these motion features, and X̅, Y̅ are the average values of these motion features, in their corresponding time windows.

For a given motion feature pair, samples of the low dose XA feature falling into a fixed time window may be identified. The window may be compared with all time windows of the same length from the corresponding high dose XA feature. Between each time window from the high dose XA feature and the time window from the low dose XA feature, a time shift may be identified. The time shift may, in some examples, be identified as the order of each time window of the high dose XA feature relative to the full time window of the same high dose XA feature. In some examples, the order of a time window of the high dose XA feature may be determined by the order of its last feature sample relative its full time window. The comparison may be performed in terms of the correlation coefficient in the NCC equation. The process may be performed for one or more combinations of motion features from high and low dose XAs to determine one pair of high and low dose XA features having a maximum correlation coefficient. According to some examples, the process may be performed for one, some, most, or all combinations of motion features from high and low dose XAs to determine one pair of high and low dose XA features having a maximum correlation coefficient. In some examples, the optimal time shift may be determined as the time shift between time windows of high and low dose XA features having the maximum correlation coefficient.

The NCC may be used to identify motion features in pairs of low and high dose XAs that have the maximum cross-correlation. For example, in block 906, the correlation coefficient may be compared to a threshold. If the correlation coefficient is below the threshold, the initial search may be indicated as a failure 918. If the correlation coefficient is above a threshold, the initial search may continue. According to some examples, the threshold may be between about 0.6-0.8.

In block 908, the motion features used to determine the correlation coefficient may be compared. If the motion features in the low dose and high dose XA are the same type of motion feature, the initial search may be completed, and an initial time shift 916 may be determined. If the motion features in the high and low dose XAs used to determine the correlation coefficients are different types of motion features, the initial search may proceed to block 910.

In block 910, a second motion feature of the high dose XA motion features may be selected. The selected second motion feature of the high dose XA motion features may be of the same type as the selected low dose XA motion feature. The selected first and second features of the high dose XA may be used to determine a phase shift offset caused by the mismatch in the motion feature types. Low dose XAs typically include less information as compared to high dose XAs. To account for this, the initial search 802 expands the usable feature pairs such that the success rate of the initial phase correlation is increased. The estimated time shift may, in some examples, include additional drift in examples where the high dose and low dose XA features are different. The additional drift may bias the motion phase correlation result. To account for the bias, potential drifts may be estimated, using a second NCC, between the selected high dose XA features and then offset from all time shifts estimated from the initial search 802 and online update 812.

In block 912, cross correlation, or correlation coefficient, for the selected high dose XA motion features may be determined. The cross correlation may be determined using the second NCC. The second NCC may be, in some examples, the same as the NCC equation provided above or may be a different NCC. In addition to the NCC, other metrics, such as various distance-based metrics may be used to temporally correlate the high and low dose XA, or estimate the phase shift offset between different types of motion features as in blocks 904 and 912.

In block 914, the correlation coefficient determined using the second NCC, e.g., a second correlation coefficient, may be compared to a threshold. If the second correlation coefficient is below the threshold, the initial search may be indicated as a failure 918. In such examples, a second search may be performed including a subset of features from block 904. In some examples, the second search may only use the same type of high and low dose XA features, thereby skipping blocks 910-914. If the second correlation coefficient meets or exceeds the threshold, the initial time shift 916 may be determined. At each of block 904 and 912, a time shift that maximizes the corresponding NCC may be obtained. The initial time shift may then be determined as the difference between these two time shifts, e.g., the time shift at block 904 and the time shift at block 912. According to some examples, the time shift determined from the second NCC may be used as an offset and subtracted from the time shift determined from the first NCC. In some examples, the initial time shift accepted in block 908 may be a case where the time shift from block 912 is 0. The online update 812, illustrated in FIG. 8, may iteratively filter the initial time shift.

According to some examples, the high-dose XA motion features may have a finite sample size. One heart cycle from the initial search may be determined and high-dose XAs from within this cycle may be used and/or included as the map for the dynamic visualization. The starting time of the heart cycle may be determined as the initial time shift. In some examples, the length of the cycle may be determined as the heartbeat period, as described with respect to FIG. 11.

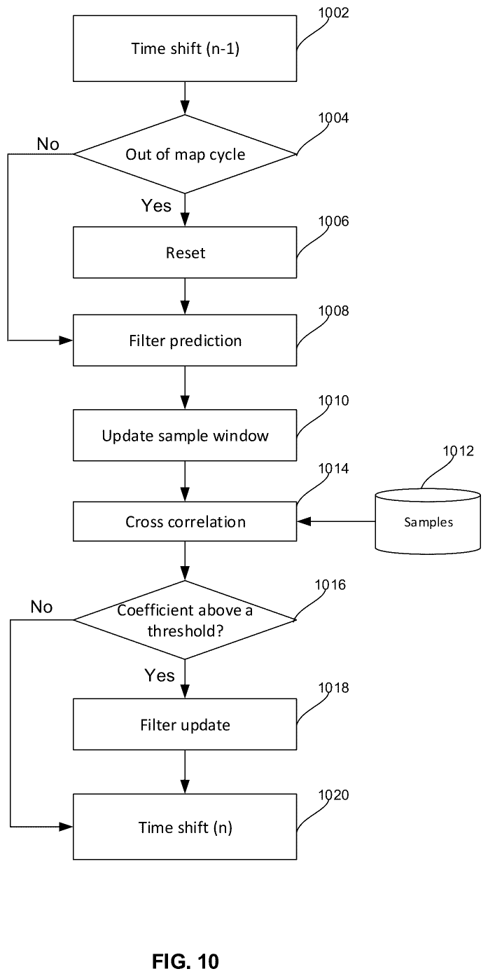

FIG. 10 illustrates an example method for performing the online update. The online update may be performed to determine the time shift. The following operations do not have to be performed in the precise order described below. Rather, various operations can be handled in a different order or simultaneously, and operations may be added or omitted.

In block 1002, the initial time shift is received. The initial time shift may be represented as “n−1”. The online update may be performed iteratively. For example, the online update may take the time shift obtained at time n−1 and estimate the time shift at time n. The “n” may denote the n-th low-dose XA motion feature sample during PCI. The initial time shift may, in some examples, correspond to n=1.

According to some examples, the maximum NCC between the low dose and high dose XA features may decrease over time, which may lead to errors in matching low and high dose XAs. In some examples, the ongoing process of low dose XA acquisition may result in the number of high dose XA available for correlation to be depleted if previous high dose XAs are not reused. To account for this, when correlating the high and low dose XAs temporally, a single heart cycle of the high dose XAs frames may be used. The low dose XAs from the same phase of distinct heart cycles may then be correlated with the same high dose XAs taken from a single heart cycle.

In block 1004, a determination may be made regarding whether the new low-dose XA is out of map cycle. For example, for each new low-dose XA sampled during PCI, if the time shift, e.g., “n−1”, is larger than the initial time shift by more than one heartbeat period, the low-dose XA may be considered out of the map cycle. If the new low dose XA is out of map cycle, then the online update may proceed to block 1006. If the new low dose XA is not out of map cycle, then the initial time shift may be input to block 1008 for filter predict.

In block 1006, the time shift may be reset by offsetting the heartbeat period from time shift “n−1”. In some examples, the time shift may be reset using other criteria. This time shift may then be input to block 1008 for filter predict.

In block 1008, a filter may be used to predict a new time shift. The filter may be, in some examples, a linear Kalman filter. The new time shift may be proposed to correlate a new low dose XA frame with the next available high dose XA frame in time order. The new low dose XA frame may be, for example, the most recently obtained low dose XA frame.

In block 1010, the sample window may be updated. In some examples, the sample window may be updated by removing the earliest low dose XA feature sample from and adding the most recent low dose XA feature sample to the last sample window. The optimal time shift may be the one that maximizes NCC and this is detailed in 1014. Time shift correction is detailed in 1018.

In block 1014, cross correlation is used to refine the time shift based on the updated sample window. For example, the time shift may be refined by moving the time window of the low dose XA motion feature until a maximum NCC is identified.

The cross correlation may generate a correlation coefficient. In block 1016, the correlation coefficient may be compared to a threshold. If the correlation coefficient is below the threshold, the time shift may be determined 1020, without updating the filter. If the correlation coefficient meets or exceeds the threshold, the filter may be updated 1018.

In block 1018, the filter may be updated when the correlation coefficient meets or exceeds the threshold. The filter may be, for example, the linear Kalman filter. According to some examples, the time shift may be updated using the following Kalman filter equation: \hat{x}ₙ=\hat{x}′ₙ+Kₙ(zₙ−\hat{x}′ₙ), where \hat{x}′ₙ is the predicted new time shift (block 1008), zₙ is the time shift optimized by the NCC (block 1014) and Ky is the Kalman gain balancing the prediction and NCC measurement. The filter update 1018 may be used to reduce the prediction uncertainty.

In block 1020, the determined time shift may be provided after the filter has been updated.

Referring back to FIG. 6, the live multiscale motions features may be used to correlate the motion phases of the high and low dose XAs spatially. The motion feature bank 602, identified working vessel 614, live multiscale motion features 606, and device segmentation 610 may be used to spatially correlate the high and low dose XAs. The spatial correlation may be used in addition to, as an alternative of, or as a refinement of the temporal correlation. For example, a spatial fitness between spatial landmarks of the low dose XAs and the working vessel of the high dose XAs may be evaluated and used to refine the temporal correlation of the high and low dose XAs.

Intrinsic motion states of the heart vessel in high and low dose XAs may also be captured using AI-based approaches. In some examples, these intrinsic motion states may be captured directly from XAs by using a convolutional neural network to extract deep features. These deep features may be used in addition to, as an alternative of or as refinement of the multiscale motion features to correlate the high and low dose XA images using temporal, spatial based metrics, or their combination.

According to some examples, for each low dose XA, given a correlated high dose XA, a loss function may be determined. The loss function may be, in some examples, a distance transform value. If the correlation between the high and low dose XAs is substantially accurate, the spatial loss may be a low value, as compared to a spatial loss value when the correlation between the high and low dose XAs is not accurate. A low loss value may correspond to the correlation of spatial landmarks and the working vessel. If the spatial mismatch is noticeable, e.g., the loss value is high, the correlation between the high and low dose XAs may be determined to be inaccurate.

In some examples, the system may automatically provide a correction when the correlation between the high and low dose XAs is inaccurate. For example, the system may provide for output correlated frames from the most recent accurate correlation. In some examples, the system may not show the current frame due to the inaccurate correlation. In another example, the system may automatically perform spatial registration to identify a correlated phase frame.

According to some examples, one or more landmarks along the working vessel may be identified. The landmarks may be automatically identified and/or identified based on a received input. For example, the system may receive an input via the computing device, touch screen, etc. corresponding to the selection of landmarks. The landmarks may correspond to a working zone or a device landing zone. The landing zone may correspond to coordinates relative to the catheter tip. In some examples, the distance between the landmarks and the catheter tip may be determined in each high dose XA. Pixels along the working vessel trajectory within distance range, e.g., the distance between the landmarks, may correspond to the determined landing zone.

The spatial correlation and/or temporal correlation of the high and low dose XAs may be fused 618 to provide dynamic visualization 620 of the delivery of a device within the vessel. For example, the system may provide for output the spatially and/or temporally correlated low and high dose XAs. The output may include any of a vessel map, live tracking of the location of the device, an indication of a treatment zone, an indication of the position of the device with respect to the treatment zone, or the like. In some examples, the output may include a plurality of images, videos, video stills, or the like. For example, two or more images and/or videos may be output relative to each other. One image may be a vessel map while another image may be a live image tracking the location of the device. The output may, in some examples, allow for a physician to dynamically visualize the delivery of a device within a vessel using less contrast. As dynamic visualization correlates high and low dose XAs, dynamic visualization may use less contrast as compared to having to continuously inject contrast to be able to visualize the vessel.

### Marker Detection and Tracking

Device marker detection and tracking may allow for the device to be dynamically visualized during the treatment stage of PCI. The device may be represented by its distinct radiopaque markers. The detection and tracking of the markers may be performed by iteratively estimating the position of the markers and the motion state in the low dose XAs. Iteratively estimating the marker position allows for detection and tracking to be performed accurately and quickly. Moreover, the process can recover from errors quickly.

According to some examples, the device may be a balloon device. However, the device may be any device, such as an imaging probe, pressure wire, stent delivery device, a vessel prep device, such as an atherectomy device or lithotripsy device, or the like. Therefore, the example provided herein with respect to a balloon device is just one example and is not intended to be limiting. The balloon device provided in this example may be replaced with any device having radiopaque markers or markers that are visible in XAs.

FIG. 12 is an example method for detecting and tracking markers on a device. Low dose XAs 1202 may be captured. In some examples, the low dose XAs 1012 may be captured live, such as during the delivery of the device.

To predict the position of the marker in the respective low dose XA, the motion of the marker in the most recent sampling period may be estimated using an optical flow estimate 1208. Optical flow assumes the local intensity of moving objects in an image to remain approximately constant for at least a short time duration. According to some examples, the optical flow equation may be: I(x,t)≠I(x+δx, t+δt), where δx is the displacement of local image region at location x from time t to t+δt. The aforementioned equation may be applied to each pair of low dose XAs sampled at an interval of δt. By applying the optical flow equation for each detected marker's position, δx may be determined and used as the motion of the marker. According to some examples, δx may be determined from the optical flow equation using the iterative Lucas-Kanade (ILK) method. In some examples, δx may contain a horizontal motion component u and a vertical motion component v. The motion estimates may be used to predict the position of the marker in the most recent low dose XA. According to some examples, a filter, such as a linear Kalman filter 1210, may be used to predict the position of the marker based on the motion estimate.

To update the position of the marker in the respective low dose XA, a neural network, such as a V-Net convolution neural network, may be used to detect marker candidates from the same low dose XA. The V-Net marker detector 1204 may be trained using annotated XAs. The XAs may be annotated to identify marker positions of devices within the XA. According to some examples, the V-Net marker detector 1204 may be trained based on annotated marker positions of an intraluminal imaging probe, such as an OCT probe, in XAs. Additionally or alternatively, the marker detector 1204 may be trained based on annotated marker positions of another device, such as a balloon delivery device, in XAs. Training the marker detector 1204 based on marker positions of any type of device, such as an OCT probe, may allow for the marker detector 1204 to detect markers from a different type of device, such as a balloon delivery device. According to some examples, the high and low dose XAs in the training data may be separated to adopt a transfer learning strategy. The marker detector 1205 may be trained to predict a probability map measuring the membership of each pixel to the marker class. According to some examples, the V-Net model may predict each position on the low dose XA image's probability of being from the marker. The “membership” may correspond to whether a position on the XA image is from the marker or not. The higher the probability value (over a scale of 0-1), the more likely the position on the XA is to be from the marker.

According to some examples, when executing the marker detector 1204, the marker detector 1204 may receive low dose XAs 1202 as input. The marker detector 1204 may output a probability map measuring the membership of each pixel to the marker class. According to some examples, the output of the marker detector 1204 may be processed to integrate the model inference into the Kalman filter 1210.

The processing may include detecting center points 1206. The center points may represent the detected markers. For example, the probability map output by the marker detector 1204 may be thresholded to segment candidate marker regions. Center points of each candidate region may be extracted. The extracted center points of each region may correspond to a detected marker.

FIG. 13 illustrates an example method for marker tracking with a V-Net model inference. The following operations do not have to be performed in the precise order described below. Rather, various operations can be handled in a different order or simultaneously, and operations may be added or omitted. FIG. 13 can be understood as an alternative method to the method described in connection with FIG. 26. Specifically, FIG. 13 describes detection and tracking method aimed at obtaining an accurate location of device markers, whereas FIG. 26 describes a detection and tracking method aimed at increasing the local image contrast between device markers and the background.

According to some examples, to reduce false positives, after the prediction step of each Kalman filter iteration, the predicted marker position 1302 may be compared to the detected marker positions 1304 determined by the marker detector 1204. The comparisons may be assigned a unique candidate marker position. According to some examples, to determine the assignment of the marker position, a pairwise distance 1306 may be evaluated based on the set of predicted and detected marker positions 1302, 1304. The pairwise distance may be, in some examples, an Euclidean pairwise distance.

The predicted position may then be sorted 1308 in ascending order based on their distances to the nearest detected positions. A filter update, such as a Kalman filter update, may be iteratively performed in the sorted order 1310. The filter update is performed on each predicted position with the nearest detected position. To avoid conflict, assigned detected marker positions and those in a surrounding region may not be considered for new assignments.

If the distance between a predicted marker position and its nearest detected marker position is greater than a threshold distance 1312, no further updates to the position may be performed 1318. If the distance between a predicted marker position and its nearest detected marker position is less than the threshold distance 1312, the neighboring, or nearby, positions may be cleared 1314. The filter may be updated 1316 based on the cleared neighbors. The marker position 1318 may then be determined.

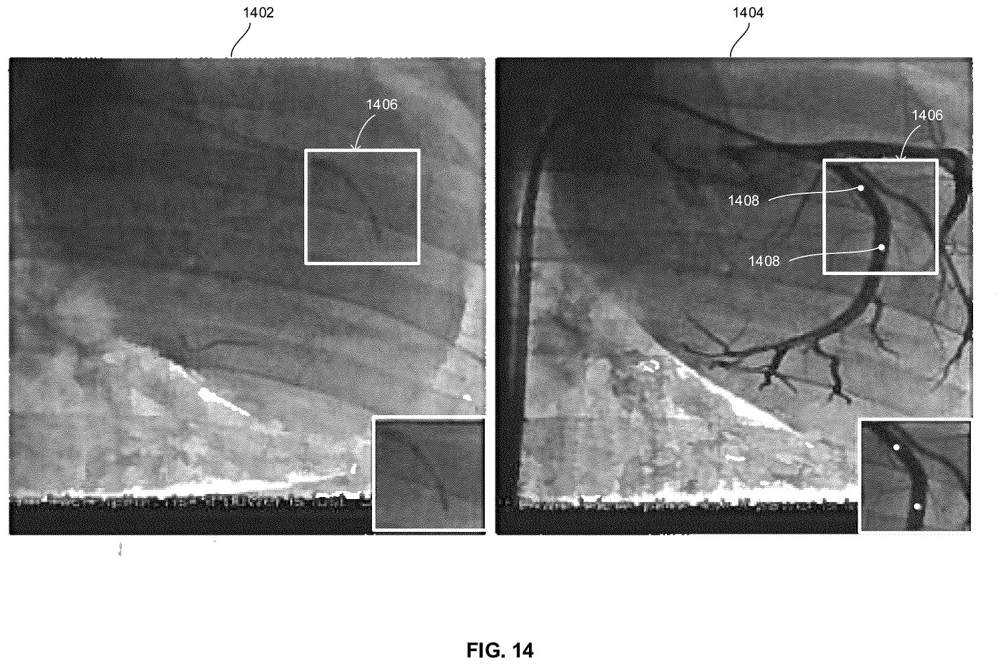

FIG. 14 illustrates the results of marker detection and tracking. Frame 1402 illustrates a real-time low dose XA and frame 1404 illustrates a selected high dose XA. As described above and herein, the high dose XA may be used to generate a vessel map and/or to identify the working vessel. The real-time low dose XA and high dose XA may be correlated using methods described above and herein to provide dynamic visualization of the device during delivery of the device within the vessel. The markers 1408 on the device may be identified in either or both the low and high dose XAs 1402, 1404 in real-time. According to some examples, an indication of the markers 1408 may be provided for output in one or both of the low and high dose XAs 1402, 1404.

According to some examples, a bounding box 1406 may be provided for output. The bounding box 1406 may be sized based on the type of device. The bounding box 1406 may define the region of the device. The region of the device may be determined based on the detected marker positions. The markers 1408 detected in the real-time low dose XA 1402 may be superimposed on the high dose XA 1404 to provide for dynamic visualization of the location of the device within the working vessel.

### Example Methods

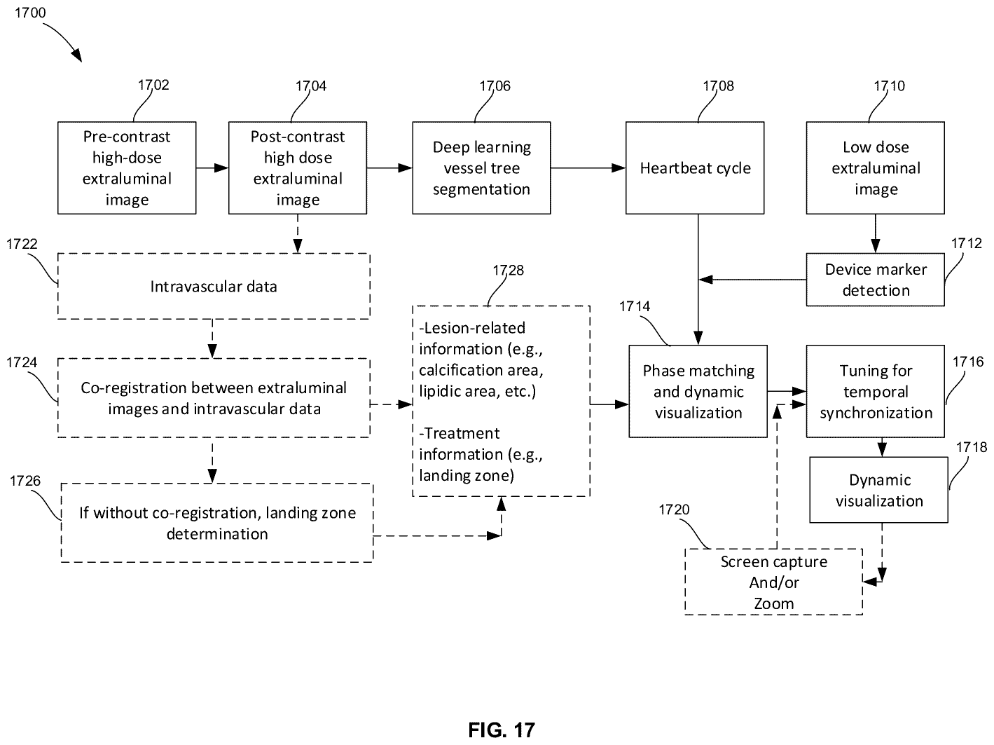

FIG. 17 illustrates an example flow chart for dynamically visualizing the delivery of a device within a vessel. The following operations do not have to be performed in the precise order described below. Rather, various operations can be handled in a different order or simultaneously, and operations may be added or omitted.

The workflow 1700 may be, for example, used as part of an extraluminal image driven PCI. The extraluminal images may be angiography images. The workflow 1700 may begin by obtaining pre-contrast high dose extraluminal image(s) 1702 and post-contrast high dose extraluminal image(s) 1704. Pre-contrast high dose extraluminal images may be, for example, extraluminal images obtained, or captured, prior to contrast being injected into the vessel. Post-contrast high dose extraluminal images may be, for example, extraluminal images obtained, or captured, after contrast is injected into the vessel. Post contrast high dose extraluminal images may be captured when the contrast is in the vessel being imaged.

According to some examples, the workflow 1700 may include obtaining intravascular data 1722. Intravascular data 1722 may be obtained using an intravascular device, such as an OCT probe, IVUS catheter, micro-OCT probe, NIRS sensor, OFDI, pressure wire, flow meter, or the like. The intravascular data 1722 may include, for example, intravascular images, pressure measurements, flow measurements, etc.

The intravascular data 1722 may be co-registered 1724 with the extraluminal images, such as pre- and/or post-contrast high dose extraluminal images 1702, 1704.

According to some examples, if intravascular data 1722 is not co-registered with extraluminal images, the landing zone may be determined 1726. According to some examples, the landing zone 1726 may be determined by a user, such as a physician. In some examples, an occlusion or other disease-related features may be identified in the post-contrast angiogram. The occlusion or disease-related feature may, in some examples, be automatically identified based on vessel data captured by an intraluminal and/or extraluminal device. For example, an occlusion or disease-related feature may be identified using a constrained contrast flow in the post-contrast extraluminal image. In some examples, the system may receive an input via the GUI corresponding to the identification of the occlusion or disease related feature. The identified occlusion or other disease-related feature may be when determining a landing zone.

The co-registered extraluminal images and intravascular data 1724 and/or the landing zone determination 1726 may be used to determine information 1728 related to lesions, treatments, etc. The information related to the lesions may include, for example, a calcification area, lipidic area, plaque length, plaque location, percent stenosis, etc. The information related to a treatment may include, for example, identifying one or more candidate landing zones for a stent, balloon, vessel prep device, or the like. The information 1728 may, in some examples, include plaque burden, fractional flow reserve (“FFR”) measurements at one or more locations along the vessel, calcium angles, EEL detections, calcium detections, proximal frames, distal frames, EEL-based metrics, stent decisions, scores, recommendations for debulking, recommendations for subsequent procedures, or the like.

A deep learning model 1706 may be applied to the post-contrast high dose extraluminal images to obtain the vessel tree morphology. The deep learning model may be, for example, an SVS-Net convolution neural network. The deep learning model may be used to segment the vessel tree from the high dose extraluminal images. The vessel tree may include, for example, the working vessel, side branches, and any other vessel of the like. The deep learning model 1706 may be trained using annotated extraluminal images. In some examples, the annotations may be done automatically by using another pre-trained neutral network. The pre-trained neural network may be, for example, the same SVS-Net neural network configured for a different software or hardware.

The deep learning model 1706 may be trained to predict a probability map measuring the membership of each pixel to the vessel tree class. Membership may, for example, correspond to whether a position on the extraluminal image is from the vessel tree or not. For example, the deep learning model may predict the probability of each position on the high dose extraluminal image as being from the vessel tree. According to some examples, a greater probability value indicates that the position on the high dose extraluminal image is more likely to be from the vessel tree. In contrast, a lower probability value may indicate that it is less likely for the position on the high dose extraluminal image to be from the vessel tree. The scale of probability values may be, for example, zero to one (0-1). In such an example, a greater probability value may be a value closer to one (1) whereas a lower probability value may be a value closer to zero (0).

According to some examples, the scale can be from 0-100, 12-22, etc. Additionally or alternatively, a lower probability value may indicate that that the position is more likely to be from the vessel tree and a higher probably value may indicate that the position is less likely to be from the vessel tree. Accordingly, a scale of 0-1 with a higher probability indicating that the position is more likely and a lower probability indicating that the position is less likely is just one example and is not intended to be limiting.

The vessel tree may be filtered. For example, the vessel tree may be filtered by prior knowledge of the vessel morphology. The vessel morphology may be probed from pre-contrast extraluminal images and propagated to subsequent post-contrast extraluminal images. In some examples, a deep learning model, such as the MaskTrack+Vnet model 310, may be used to propagate the vessel information.

The output of the deep learning model 1706 may be, for example, a segmented vessel tree. The segmented vessel tree may exclude the regions from the guide catheter. In some examples, the segmented vessel tree may include the regions from the intraluminal imaging catheter. The regions from the intraluminal imaging catheter may be detected from the pre-contrast extraluminal images. The regions may include, for example, the portion of the image frame that include the imaging catheter but not the guide catheter. According to some examples, the output of the deep learning model 1706, e.g., the segmented vessel tree, may be used for dynamic visualization.

The heartbeat cycle 1708 may be determined. The high dose extraluminal sequence obtained prior to the insertion of device and/or prior to PCI may be cached for fast access during the treatment phase. The treatment phase may include, for example, the delivery of a device in the vessel, such as a stent, balloon, or the like. The sequence of extraluminal images may cover at least one cardiac cycle. The length of the cardiac cycle may be determined using heartbeat estimation. An example of heartbeat estimation is described with respect to FIG. 11.

According to some examples, heartbeat estimation may use a multi-scale feature bank, such as multiscale motion features 1102. The multi-scale feature bank may include motion features at the working vessel level 702, wire tip level 704, vessel pixel level 706, or any other levels manifesting the heartbeat. In some examples, the motion features may be absolute and relative motion of the working vessel, guide catheter tip, distal wire tip, intermediate points along the working vessel, or the like. The absolute and relative motion features may correspond to, for example, the coordinates and the optical flow of a point in the high dose extraluminal image and/or low dose extraluminal images.

An ACF, such as the ACF 1104 described in FIG. 11, may be applied to multi-scale features to estimate the heartbeat. In some examples, peaks and valleys from the ACF of the motion features may be identified. Separations in the peaks and valleys may be quantized and filtered, such as by filter 1110. According to some examples, filtering of the peaks and valleys may be implemented using a NMS, such as NMS 1108. Additional or alternative filters, such as a median filter or other statistical filter, may be used to identify the heartbeat period.

As part of the workflow 1700, low dose extraluminal images 1710 may be obtained. Low dose extraluminal images 1710 may be captured during the delivery of an intravascular device. The low dose extraluminal images 1710 may be, for example, fluoroscopic images. According to some examples, the low dose extraluminal images 1710 may substantially correspond to frames 324 in FIG. 3A and/or frames 1202 in FIG. 12.

The low dose extraluminal images 1710 may be used to detect the device marker 1712. The device may be, for example, a balloon device, an imaging probe, a pressure wire, a stent delivery device, a vessel prep device, such as an atherectomy device or lithotripsy device, or the like. The device may include radiopaque markers or markers that are visible in extraluminal images.

The markers on the device may be detected 1712 using artificial intelligence, such as a AI model, a convolution neural network, or the like. For example, a V-Net convolutional neural network, the MaskTrack+Vnet model, or the like may be used to detect the marker candidates from the same low dose extraluminal images. According to some examples, the V-Net marker detector may be trained based on annotated marker positions of an intraluminal device, such and an intraluminal imaging probe or balloon delivery device, in extraluminal images. The high and low dose extraluminal images in the training data may be separated to adopt a transfer learning strategy.

The marker detector, e.g., the V-Net model, may be trained to predict a probability map. According to some examples, the marker detector may be trained to predict the probability of the position on the low dose extraluminal image is a marker. In some examples, the probability map may be thresholded to segment the device markers. Thresholding may include, for example, segmenting the pixels inside the device markers based on the image's probability map. For example, the marker detector may predict a map of each pixel's probability of being inside the device marker. The device marker center point coordinates may, in some examples, be determined during post-processing of the image based on the thresholded probability map. For example, the device marker center coordinates may be determined by identifying connected regions and detecting the center points of each region. The marker positions may be propagated from previous frames using the motion estimates of the markers. In some examples, the motion may be determined from optical flow. According to some examples, the propagated device marker positions from previous frames may be updated by the neural network-detected marker positions in the current frame. For example, a linear Kalman filter may be used to update the device marker positions. Details pertaining to how the marker is detected and tracked are discussed in more detail with respect to FIGS. 12 and 13.

According to some examples, the detected markers 1712 may be enhanced. For example, the detected markers may be enhanced to increase the visualization of the markers on the low and/or high dose extraluminal images. Enhancing the markers may increase the visualization of the markers by differentiating the markers in terms of contrast, color, etc. as compared to surrounding image features. This may make it easier for the user to identify the markers and focus on a given area of the high and/or low dose extraluminal images provided for output as part of the dynamic visualization.

The detected markers may be enhanced using local image processing. In some examples, the local image processing may be through local contrast stretching. Various intensity transformation-based methods may be applied to stretch local image contrast.

In examples where intravascular data 1722 is obtained and information 1728 is determined, the information 1728, heartbeat cycle 1708, and detected device markers 1712 may be phase matched to provide dynamic visualization 1714.

Phase matching may include, for example, matching the phase of the high dose extraluminal images 1702, 1704 and the low dose extraluminal images 1710. The phase matching may be done using motion features. For example, the high and low dose extraluminal images may be temporally phase matched without the use of EKG or other physiological signals that measure heartbeat or breath patterns of a patient or time stamps. Rather, the temporal phase matching may be based on image motion features. According to some examples, the high and low dose extraluminal images may be spatially phase matched. Details pertaining to how the high and low dose extraluminal images are phased matched are discussed in more detail with respect to FIGS. 8 and 9.

Dynamic visualization may include, for example, providing as output a road map image relative to the low dose extraluminal image. The road map image may be, for example, a vessel map. The detected markers may, in some examples, be provided for output on the vessel map and/or the low dose extraluminal image to provide an indication of the location of the device in real time, without having to inject additional contrast. According to some examples, the information 1728 may be provided for output on or relative to the vessel map and/or low dose extraluminal image. For example, a treatment zone, initially identified on the vessel map, may be provided for display on the low dose extraluminal image such that the location of the device on the low dose extraluminal image can be seen relative to the treatment zone. In some examples, the marker may be provided for output on the vessel map to provide an indication of the location of the device in real time with respect to the treatment zone.

According to some examples, the temporal phase matching may have to be tuned. Tuning the phase matching may include, for example, adjusting, selecting, or matching another high dose extraluminal image with the low dose extraluminal image. Tuning the temporal synchronization 1716 may account for errors that may occur during automatic temporal phase matching. For example, if the patient has an irregular heartbeat, goes into an irregular rhythm during the procedure, or the like, tuning for temporal synchronization 1716 may be used to account for these differences.

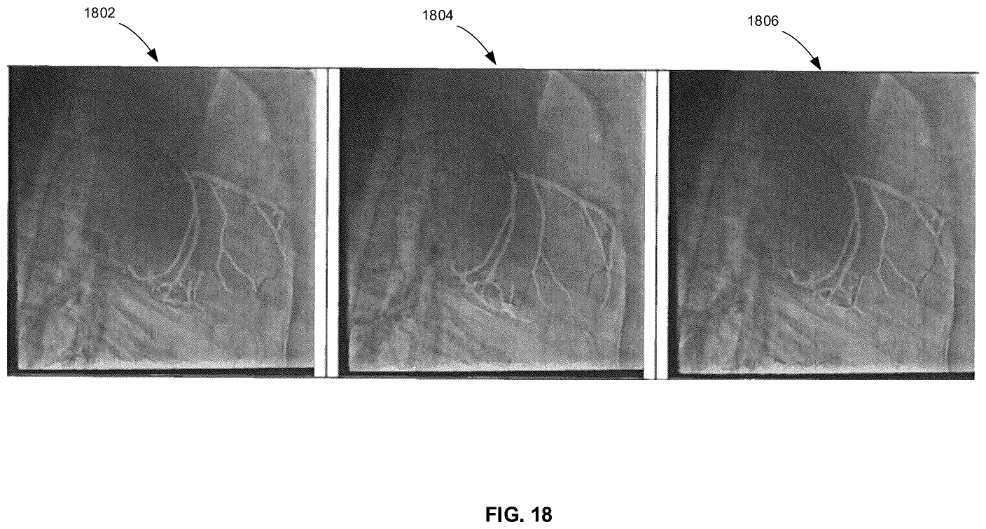

FIG. 18 illustrates an example of tuning the temporal synchronization of the high and low dose extraluminal images. Frame 1802 illustrates the segmented vessel tree provided by the deep learning model 1706 as an overlay on the low dose extraluminal image 1710. The segmented vessel tree in frame 1802 was automatically temporally matched 1714 with the low dose extraluminal image.

In frame 1804, the temporal synchronization of the vessel tree and the low dose extraluminal image was tuned. For example, the selected vessel tree may be two frames ahead of the vessel tree from the phase matching 1714. In frame 1806, the selected vessel tree may be five frames ahead of the vessel tree from phase matching 1714.

According to some examples, the tuning may be done by the user, such as a physician. The physician may select a vessel tree from a heartbeat cycle, a specific point in the heartbeat cycle, etc. In some examples, a vessel tree may be chosen as a static image such that the vessel tree provided for output does not change regardless of the portion of the heartbeat cycle. In such an example, the low dose extraluminal image, e.g., the live fluoroscopic image, may align with the static vessel tree during at least a portion of the heartbeat cycle. In some examples, the tuning 1716 may allow for the static vessel tree to be adjusted or changed. According to some examples, the tuning 1716 may be done using various user control interfaces, such as a knob control, keyboard, joystick, touch screen, or the like.

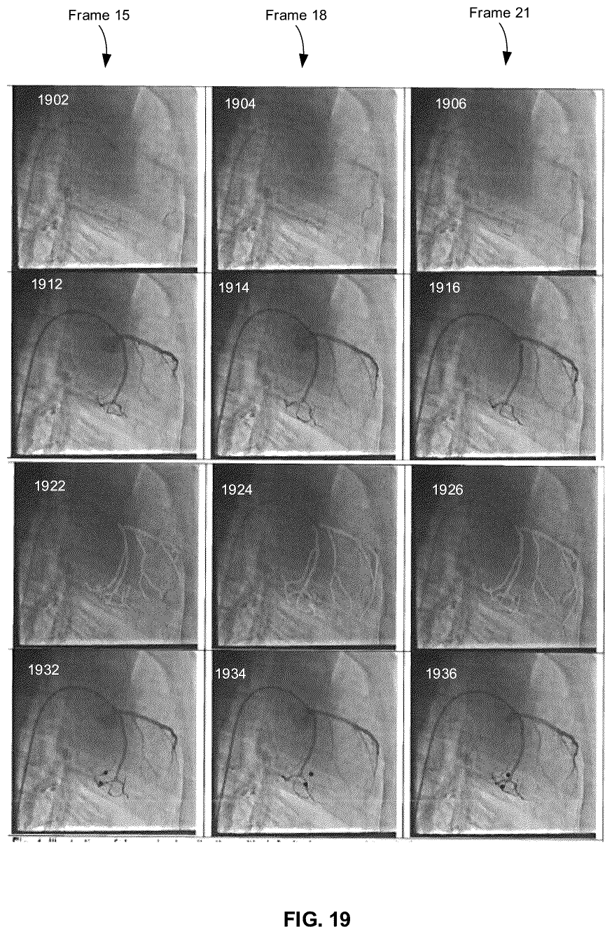

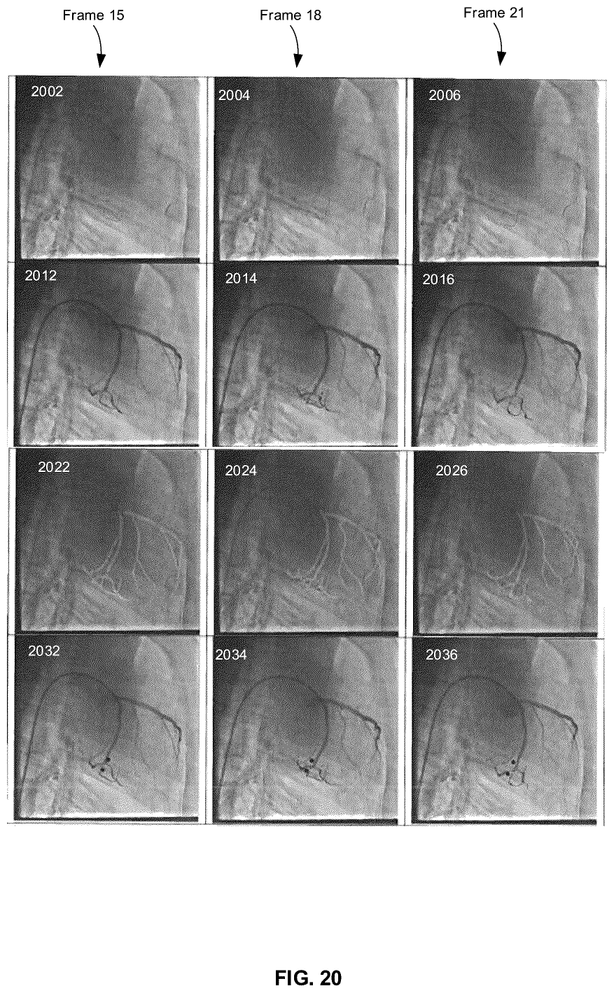

FIGS. 19 and 20 illustrate another example of tuning the temporal synchronization of the high and low dose extraluminal images. FIGS. 19 and 20 illustrate three frames of the low dose extraluminal images in different cardiac phases of the same cycle and the corresponding dynamic visualization elements with phase matching 1714 and default phase matching followed by tuning 1716. As shown in the image frames, the high dose and low dose extraluminal image sequences exhibit significant differences, which may be due to rewiring. In such an example, the initial phase matching 1714 may not adequately synchronize the cardiac phase in the sequences of high and low dose extraluminal images. With subsequent tuning 1716 of the high dose extraluminal images to adjust the initial high dose extraluminal image of the sequence, the high and low dose extraluminal image sequences may be more accurately synchronized.

Referring to FIG. 19, as an example, image frame 1902 illustrates a low dose extraluminal frame of the sequence of frames, e.g., frame 15, image frame 1904 illustrate another low dose extraluminal frame of the sequence of frames, e.g., frame 18, and image frame 1906 illustrate yet another low dose extraluminal frame of the sequence of frames, e.g., frame 21. Image frame 1912 illustrates frame 15 phase matched with a high dose extraluminal frame using phase matching 1714, image frame 1914 illustrates frame 18 phase matched with a high dose extraluminal frame using phase matching 1714, and frame 1916 illustrates frame 21 phase matched with a high dose extraluminal frame using phase matching 1714. Image frame 1922 illustrates an overlay of the vessel tree segmented from the high dose extraluminal images overlaid on frame 15, image frame 1924 illustrates an overlay of the vessel tree segmented from the high dose extraluminal images overlaid on frame 18, and image frame 1926 illustrates an overlay of the vessel tree segmented from the high dose extraluminal images overlaid on frame 21. Image frame 1932 illustrates an overlay of device markers detected from frame 15 on the high dose extraluminal image, image frame 1934 illustrates an overlay of device markers detected from frame 18 on the high dose extraluminal image, and image frame 1936 illustrates an overlay of device markers detected from frame 21 on the high dose extraluminal image. In this example, using phase matching 1714, the alignment of visual landmarks between the high dose extraluminal images 1702, 1704, and the low dose extraluminal images 1710 results in a poor temporal synchronization.

To improve the temporal synchronization of the example of FIG. 19, tuning 1716 may be performed. FIG. 20 illustrates an example of how tuning 1716 can improve the temporal synchronization. For example, the frames of the low dose extraluminal frames may be matched with a high dose extraluminal image in substantially the same cardiac phase. Substantially the same cardiac phase may, in some examples, correspond to the same cardiac motion. The low dose extraluminal images may be overlaid on the auto-phase matched high dose images, as illustrated in FIG. 19. However, instead of accepting the auto phase-matched overlay, for the low dose extraluminal images in the live feed, a second high dose extraluminal image may be selected and overlaid on it, as illustrated in FIG. 20. In some examples, an additional offset may be added to the frame index of each auto phase-matched high dose extraluminal image frame to specific the frame index of the second high dose extraluminal image that was used for the overlay. The offset may be cached and updated. The offset may be updated automatically and/or via user input.

In FIG. 20, image frame 2002 illustrates a low dose extraluminal frame of the sequence of frames, e.g., frame 15, image frame 2004 illustrate another low dose extraluminal frame of the sequence of frames e.g., frame 18, and image frame 2006 illustrates yet another low dose extraluminal frame of the sequence of frames e.g., frame 21. Image frame 2012 illustrates frame 15 phase matched with a high dose extraluminal frame using phase matching 1714 with subsequent tuning 1716, image frame 2014 illustrates frame 18 phase matched with a high dose extraluminal frame using phase matching 1714 with subsequent tuning 1716, and frame 2016 illustrates frame 21 phase matched with a high dose extraluminal frame using phase matching 1714 with subsequent tuning 1716. Image frame 2022 illustrates an overlay of the vessel tree segmented from the high dose extraluminal images overlaid on frame 15, image frame 2024 illustrates an overlay of the vessel tree segmented from the high dose extraluminal images overlaid on frame 18, and image frame 2026 illustrates an overlay of the vessel tree segmented from the high dose extraluminal images overlaid on frame 21. Image frame 2032 illustrates an overlay of device markers detected from frame 15 on the high dose extraluminal image, image frame 2034 illustrates an overlay of device markers detected from frame 18 on the high dose extraluminal image, and image frame 2036 illustrates an overlay of device markers detected from frame 21 on the high dose extraluminal image.

Referring back to FIG. 17, after tuning the temporal synchronization 1716, the dynamic visualization may be updated 1718 provided for output. The dynamic visualization may provide an output including any of a vessel map, live tracking of the location of the device, an indication of a treatment zone, an indication of the position of the device with respect to the treatment zone, or the like. In some examples, the output may include a plurality of images, videos, video stills, or the like. For example, two or more images and/or videos may be output relative to each other. One image may be a vessel map while another image may be a live image tracking the location of the device. The output may, in some examples, allow for a physician to dynamically visualize the delivery of a device within a vessel using less contrast. According to some examples, such as when intravascular data 1722 is obtained, the intravascular data may be co-registered and provided for output relative to the vessel map and/or low dose extraluminal image. In some examples, one or more additional representations of the intravascular data 1722 may be provided for output, such as graphs, an axial representation of the vessel, or the like. The graphs may be, for example, graphical representations of the FFR, EEL, pressure measurements, or the like. The axial representation may be a symmetrical representation of the vessel. The axial representation may be symmetrical about the longest axis, such as the longitudinal axis.

According to some examples, the output may be screen-captured and/or zoomed in 1720. The output may be automatically screen captured when the device reaches a region of interest. For example, the detected markers 1712 may be used to determine the location of the device relative to a region of interest. The region of interest may be, for example, a treatment device landing zone, a balloon device zone, a vessel prep device zone, or a lesion related zone. In some examples, the region of interest may be a selected portion of the vessel, a vessel line, or the like.

When the detected marker is within a threshold distance of the region of interest, the system may automatically screen-capture the output. The threshold distance may be determined based on a number of pixels between an outer boundary of the region of interest and the detected marker of the device. In some examples, the threshold distance may be determined based on a spatial distance, such as Euclidean distance or geodesic distance, between the detected markers and the region of interest.

According to some examples, the user, such as the physician, may provide an input to screen-capture the output. The screen-capture may be provided for output. According to some examples, the user may confirm the location of the device based on the screen-capture without having to inject additional contrast agents.

The dynamic visualization and/or the screen-capture may be manually or automatically zoomed in to display a high-resolution view of a region of interest. The region of interest may, in some examples, correspond to the location of the device, a treatment zone, or the like. According to some examples, the output of the dynamic visualization and/or the output of the screen-capture may automatically zoom in on the location of the device. In one example, the output of the dynamic visualization may automatically zoom in to focus on the device as the device approaches the treatment zone. The spatial distance between the detected markers and the treatment zone may be used to determine whether the device is approaching the treatment zone and/or has reached the treatment zone. In some examples, a number of pixels between the outer boundary of the treatment zone and the detected marker may be used to determine whether the device is approaching the treatment zone and/or has reached the treatment zone.

In some examples, a selected portion of the output of the dynamic visualization and or the output of the screen-capture, such as a region of interest, may be enhanced using local contrast stretching. Local contrast between the device markers and background may be stretched within the detected and tracked regions of interest. By selectively enhancing the local contrast between markers and surrounding regions, the model reserved computational resources as left computational resources are expended compared to enhancing the entire working vessel.

According to some examples, the pixel values within a region of interest may be populated and sorted into an approximate representation, such as a data graph or a histogram. In some examples, a threshold t on the intensity may be set by a user or by the AI model. According to some examples, the threshold t may be the pixel value corresponding to the 1.5-th percentile in the data representation. The choice of 1.5-th percentile as the threshold t is not fixed. Rather, threshold t may be varied to ensure that it stays above pixel values of the device marker. In some examples, pixels with intensity values lower than threshold t may be normalized within the interval [0, t]. A median filter may be applied to the intensity-stretched region of interest to smooth out noise exhibiting similar pixel values to that of the device markers. According to some examples, the filter size may be set to 3×3.

To ensure consistency in the background pixel value distributions inside and outside a given region of interest, the local contrast stretch may be further localized to the vicinity of the device markers. According to some examples, for each region of interest, the pixel values before and after the local contrast stretch may be cached, and the difference may be applied in a spatial-dependent manner as follows:

$$I = ( {I}^{′} - {I}_{0} ) \times W + {I}_{0} ,$$

where I₀ and I′ are the pixel values from the same region of interest before and after the local contrast stretch, and W is a spatial weighting function. According to some examples, W may be a Gaussian kernel centered at the device marker's center point in reference to the region of interest. In case multiple markers are present within a region of interest, W may be a mixture of Gaussian kernels each centered at one marker's center point. According to some examples, W may be normalized to [0, 1] to selectively apply the pixel-level intensity changes within the region of interest.

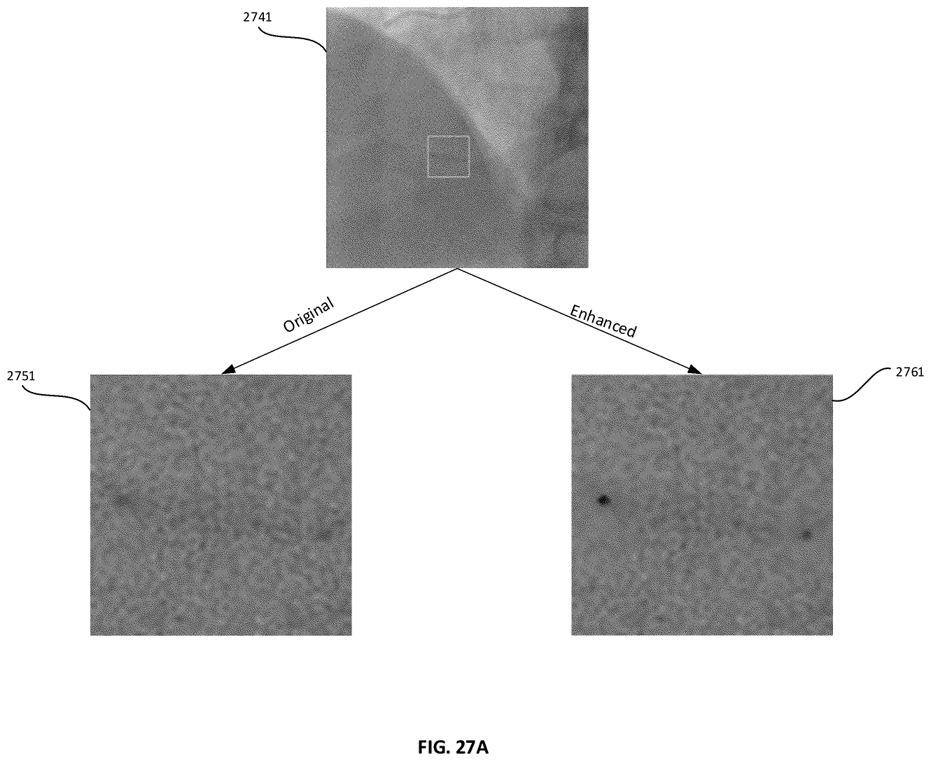

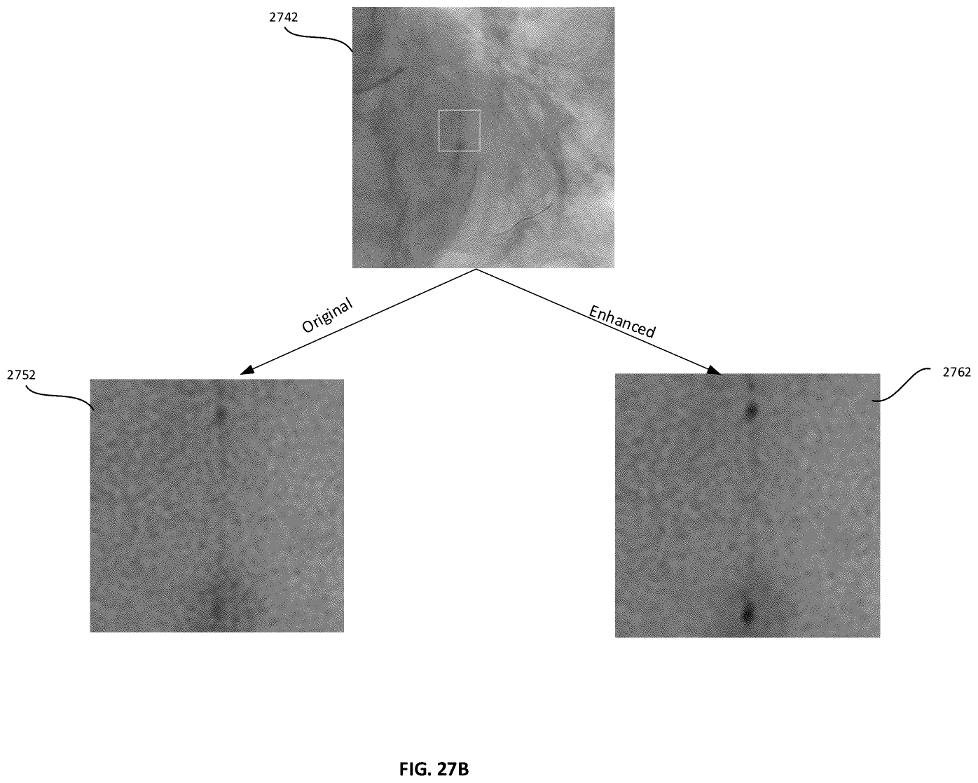

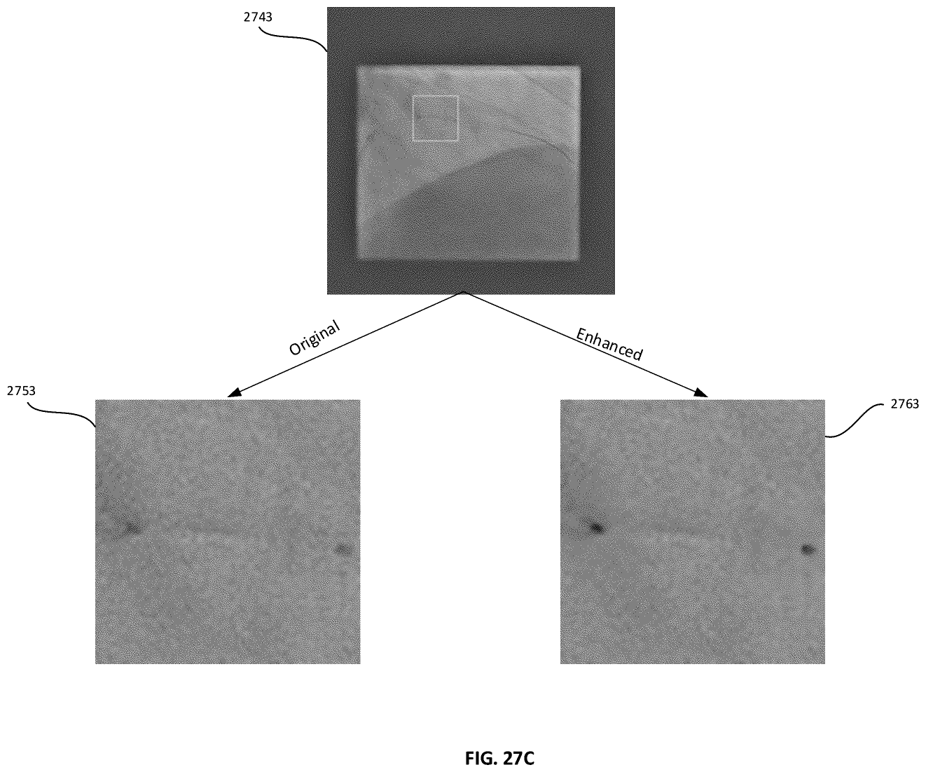

FIGS. 27A-27C illustrate the comparisons between a regular zoom and the local contrast stretch of a region of interest. Frames 2741, 2742, and 2743 are entire XA images showing the full field of view of the working vessel. In each of the frames 2741, 2742, and 2743, the region of interest is bounded by a box. In these examples, each region of interest contains two device markers. The frames are low dose XAs acquired similar to what would be captured in a clinical setting. Each frame shows a different type of working vessel. Frames 2751, 2752, and 2753 are zoomed in images of the respective regions of interest with no enhancement. Frames 2761, 2762, and 2763 are enhanced stretched images of the respective regions of interest. The enhanced frames 2761, 2762, and 2763 are magnified images of the regions of interest, enhanced using the methods described above. In the enhanced frames 2761, 2762, and 2763 the device markers are more clearly visible, as they appear darker and sharper than the corresponding frames 2751, 2752, and 2753.

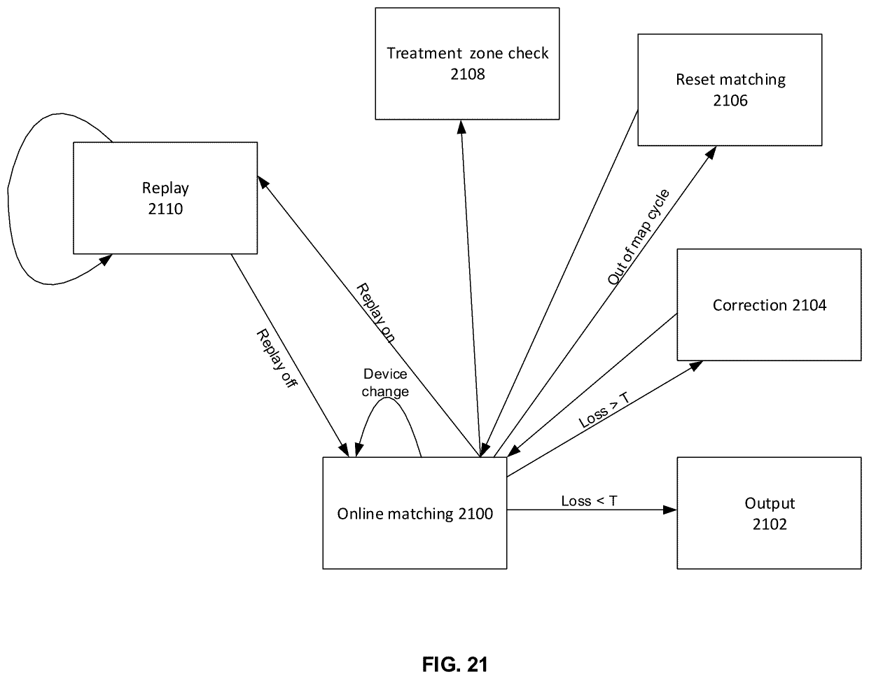

FIG. 21 illustrates an example real-time state transition. Motion stabilization, or motion compensation, may be used for online matching 2100. Online matching 2100 may, in some examples, correspond to the phase matching 1714 described in relation to FIG. 17. According to some examples, online matching 2100 may correspond to phase matching used during the live feed of the low dose extraluminal image sequence. A loss may be determined and used to evaluate errors in the matching process. The loss may be determined based on a distance transform from spatial landmarks in the low dose extraluminal images to the working vessel map in the motion stabilized and motion compensated high dose extraluminal image. The loss may be compared to a threshold “T”. In some examples, if the loss is less than the threshold, the dynamic visualization may be provided for output whereas if the loss is greater than the threshold additional correction 2104 may be needed before providing the dynamic visualization for output. The correction 2104 may include, for example, tuning 1716, as described with respect to FIGS. 17, 20, and 21. The correction 2104 may be automatically initiated and/or user initiated. For example, user initiated correction 2104 may occur based on visual feedback not captured by the determined loss. In some examples, visual feedback for the correction 2104 may be based on the closeness of spatial landmarks in the low dose extraluminal images to the working vessel map from the motion stabilized and motion compensated high dose image. The spatial landmarks may be, for example, the guide catheter tip, wire tip, balloon body, etc.

The high dose extraluminal images in the selected sequence may be phase matched, using phase match 1714 and/or tuning 1716, to the low dose extraluminal images based on their temporal order. When the selected high dose extraluminal images are used once for phase matching, the system may loop back to the first high dose extraluminal image to reset phase matching 2106. Phase matching of incoming low dose extraluminal images, e.g., live fluoroscopic images, may be repeated on the same sequence of high dose extraluminal images.

According to some examples, the system may perform a treatment zone check 2108. Treatment zone check 2108 may determine whether the detected device markers 1712 are close to a predefined treatment zone, such as a stent landing zone or a balloon landing zone. In some examples, the treatment zone check 2108 may be based on a spatial distance, such as a Euclidean distance, geodesic distance, pixel distance, or the like. If the determined distance is within a threshold distance of the treatment zone, an output may be provided. In some examples, the output may be a notification or alert. The notification may be a visual, audible, or haptic notification. The notification may indicate the closeness of the device to the treatment zone.

According to some examples, the system may detect when the low dose extraluminal images are not updated. The low dose extraluminal images may not be updated due to an interruption in image acquisition. In examples where the low dose extraluminal images are not updated, the system may enter replay mode 2110. Replay mode 2110 may include a replay period. The replay period may be, for example, the number of frames between the current low dose extraluminal image frame and the nearest previous low dose extraluminal image frame it repeats. The nearest previous low dose extraluminal image may be, for example, the most recent previously captured low dose extraluminal image frame. In some examples, the nearest previous low dose extraluminal image may be the nearest low dose extraluminal image frame based on distance, such as based on the image capture position. According to some examples, the elements of the dynamic visualization, e.g., treatment zones, device locations, etc., may be provided for output during replay mode 2110.

The system may compare the current low dose extraluminal frame with previous low dose extraluminal image frames in a given sequence. When the current low dose extraluminal image frame corresponds to at least one of the low dose extraluminal image frames in the sequence, the system may remain in replay mode 2110. The system may exit replay mode 2110 when a low dose extraluminal image frame is different from the previous low dose extraluminal frames in the sequence. In such an example, once the system exits replay mode 2110, the system may return to online matching 2100.

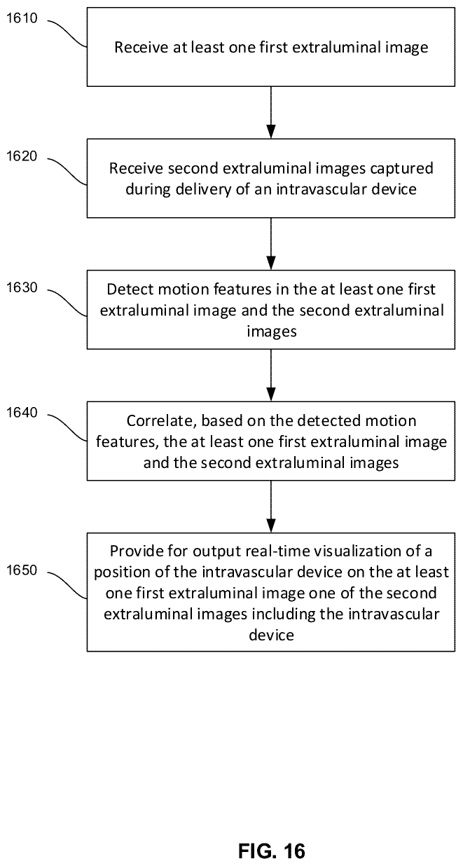

FIG. 16 illustrates an example method for dynamically visualizing the delivery of a device within a vessel. The following operations do not have to be performed in the precise order described below. Rather, various operations can be handled in a different order or simultaneously, and operations may be added or omitted.

In block 1610, at least one first extraluminal image may be received. The at least one first extraluminal image may be, for example, a high dose contrast XA. According to some examples, a vessel map may be generated based on the first extraluminal image.

According to some examples, the vessel map may include an indication of the working vessel. The working vessel may be automatically detected using AI trained to automatically detect the working vessel. Training the AI may comprise annotating pre-contrast extraluminal images as line strips following a trajectory of at least one of a guide wire or a guide catheter, labeling a path of the working vessel in post-contrast extraluminal images, providing the annotated pre-contrast extraluminal images and labeled post-contrast extra luminal images as training data to the AI model, and training the AI model to predict a vessel trajectory. According to some examples, the AI may include a AI model trained to automatically detect the working vessel.

The AI model may be executed to predict, or identify, the working vessel trajectory. For example, at least one pre-contrast extraluminal image may be received as input into the AI model. The AI model, based on the at least one pre-contrast extraluminal image, may detect a guide wire of the intravascular device. The AI model, based on the detected guide wire, may propagate wire information. The AI model may automatically predict, based on the propagated wire information, a working vessel trajectory.

In block 1620, second extraluminal images captured during delivery of an intravascular device may be received. The second extraluminal images may be, for example, low dose contrast XAs. The intravascular device may be, for example, a stent delivery device, a balloon device, an intravascular imaging probe, or a pressure wire.

The at least one first extraluminal image may be captured, or generated, at a different time than the second extraluminal images. For example, the at least one first extraluminal image may be generated during the diagnosis stage, the procedure planning stage, or the like whereas the second extraluminal images may be captured during the procedure, e.g., the delivery of the device within the vessel.

In block 1630, motion features in the at least one first extraluminal image and the second extraluminal images may be detected. The motion features may include at least one of a guide catheter tip, a distal endpoint of a working vessel, an optical flow at the guide catheter tip, or an optical flow at the distal endpoint of the working vessel. According to some examples, detecting the motion features may further comprise automatically detecting, by executing a AI model, the working vessel. In some examples, a heartbeat period of a patient may be determined based on the detected motion features.

In block 1640, the at least one first extraluminal image and the second extraluminal images may be correlated based on the detected motion features. Correlating the at least one first extraluminal image and the second extraluminal image may comprise determining vessel level motion, wire tip level motion, and vessel pixel level motion. Determining vessel level motion may comprise determining a two-dimensional translation vector. The two-dimensional translation vector may correspond to two-dimensional translation at an n-th frame with respect to a first frame. Determining the wire tip level motion may comprise determining absolute spatial information and relative spatial information. Absolute spatial information may correspond to coordinates and relative spatial information may correspond to optical flow between adjacent image frames. Determining vessel pixel level motion may comprise determining absolute spatial information and relative spatial information.

According to some examples, a spatial-temporal phase match between the at least one first extraluminal image and at least one of the second extraluminal images may be determined based on the detected motion features. For example, the detected motion features may be resampled at a common frame rate. A maximum correlation coefficient for pairs of motion features in the at least one first extraluminal image and the second extraluminal images may be determined. A time shift may be determined based on the maximum correlation coefficient. In some examples, a drift between the detected motion features in the at least one first extraluminal image may be determined. The time shift may be adjusted based on the determined drift. According to some examples, the time shift may be iteratively filtered. The time shift may be updated to a corrected time shift based on the iteratively filtered time shift.

According to some examples, the spatial-temporal phase match may be tuned. For example, another first extraluminal image different than the at least one of the first extraluminal images may be identified. The spatial-temporal phase match may be tuned based on the other first extraluminal image. The other first extraluminal image may be, for example, another first extraluminal images from the sequence of first extraluminal images. In such an example, tuning the spatial-temporal phase match may include shifting the sequence of the first extraluminal images based on the identified another first extraluminal images. The real-time visualization may be updated based on the other first extraluminal image.

In block 1650, a real-time visualization of a location of the intravascular device may be provided for output on the at least one first extraluminal image one of the second extraluminal images including the intravascular device.

According to some examples, the intravascular device may be detected in the second extraluminal images. Detecting the intravascular device may include, for example, detecting one or more markers of the intravascular device. The intravascular device and/or the markers of the intravascular device may be detected by executing a AI model. The AI model may be trained by providing, as input, a co-registration dataset comprising a plurality of intraluminal images and extraluminal images. The intraluminal and extraluminal images may be annotated images. The annotated images may include annotations identifying one or more intravascular device markers. The AI model may be trained to predict a position of the intravascular device.

According to some examples, detecting the intravascular device may include detecting an optical flow of the intravascular device. The position of the intravascular device in a first frame of the second intraluminal images may be determined based on the optical flow. The position of the intravascular device in a subsequent frame may be determined based on the optical flow. In some examples, the position of the intravascular device in the subsequent frame may be further based on the position of the intravascular device in the first frame.

In some examples, detecting the intravascular device may include detecting an optical flow of the intravascular device. The position of the intravascular device in a first frame of the second extraluminal images may be determined by executing a AI model based on at least a first frame of the second extraluminal images. The position of the intravascular device in a subsequent frame of the extraluminal images may be predicted based on the detected optical flow and the position of the intravascular device in a previous frame of the second extraluminal images. The position of the intravascular device in the subsequent frame of the second extraluminal images may be updated by executing the AI model based on at least a subsequent frame of the second extraluminal images and the predicted position of the intravascular device in a subsequent frame of the second extraluminal images.

In some examples, the output may include an indication of a treatment zone on at least one of the second extraluminal images or the at least one first extraluminal image. The treatment zone may be, for example, a stent landing zone or a balloon landing zone.

According to some examples, the system may provide, as output, a notification. The notification may be provided when the intravascular device is inserted prior to, approaching, within, or past a treatment zone.

According to some examples, a screen capture of the real-time visualization of the position of the intravascular device may be captured. The screen capture may, in some examples, be automatically captured when the intravascular device is within a threshold distance of a region of interest. The region of interest may be a treatment zone. The treatment zone may be at least one of a treatment device landing zone, a balloon device zone, a vessel prep device zone, or a lesion related zone. According to some examples, the region of interest may be automatically determined based on intravascular data captured by one or more extraluminal and/or intraluminal devices. For example, a treatment zone, such as a stent landing zone, may be automatically determined based on one or more EEL values, calcium values, plaque burden, vessel diameter values, or the like. In some examples, the region of interest may be received as an input. For example, the system may receive an input corresponding to a selection of a region of interest. The input may be, for example, a user, such as a physician, selecting locations on or along the vessel via the user interface.

Determining the threshold distance may include at least one of determining a number of pixels between an outer boundary of the region of interest and at least one detected marker on the intravascular device or determining a spatial distance between the at least one detected marker on the intravascular device and the region of interest. The spatial distance may be a Euclidean distance, a geodesic distance, etc.

According to some examples, a zoom feature may be implemented to enlarge a portion of the real-time visualization. The portion of the real-time visualization may be automatically zoomed when the intravascular device is within a threshold distance of a region of interest. The portion of the real-time visualization may correspond to the region of interest. The region of interest may be, for example, a treatment zone or a location of the intravascular device. The treatment zone may be at least one of a treatment device landing zone, a balloon device zone, a vessel prep device zone, or a lesion related zone. Determining the threshold distance may include at least one of determining a number of pixels between an outer boundary of the region of interest and at least one detected marker on the intravascular device or determining a spatial distance between the at least one detected marker on the intravascular device and the region of interest. The spatial distance may be a Euclidean distance or a geodesic distance.

The systems and methods described above and herein provide for motion stabilized treatment visualization and PCI navigation, e.g., dynamic visualization. The dynamic visualization may be implemented at different zoom-in scales, allowing for physicians to view as much or as little detail as necessary. In addition, the dynamic visualization may also be implemented at different instances during a procedure. In some examples, the system may receive an input corresponding to a request for dynamic visualization for a threshold period of time, e.g., a few seconds. The dynamic visualization may, in some examples, be used as a replacement to injecting additional contrast in order. For example, the dynamic visualization may correspond to a virtual puff that simulates and, therefore, replaces a puff that injects small amount of contrast to visualize vessels. In such virtual puff scenarios, the system would include at least some, if not all, processing steps as described above and herein to detect landmarks in low dose XAs, calculate motion features, and perform phase matching. The vessels, lesions, devices, landing zones and relevant information from the phase-matched high dose XAs are then displayed in low dose XA views. The system can activate the virtual puff in response to an input. For example, the system may include inputs that are activated by the physician, such as a button, a computer mouse, a joystick, or a virtual injector. In some examples, the virtual puff can also be triggered by following standard puff steps/devices, but without the injection or use real contrast. The period of visualization can be estimated based on look up table approaches. For example, if a predetermined amount of contrast (e.g., X ml of contrast) can last a threshold period of time (e.g., Y seconds), the visualization system can be configured to provide dynamic information for the threshold period of time (e.g., Y seconds) and then fade out. Further, the output provided by the dynamic visualization allows the intravascular device to be detected, tracked, and projected on a vessel map. This allows for physicians to be able to clearly visualize the vessel morphology while delivering an intravascular device while taking only low dose XAs. Moreover, dynamic visualization allows for the efficient and almost fully automatic fusion of information of low and high dose XAs.

The aspects, features, and examples of the disclosure are to be considered illustrative in all respects and are not intended to limit the disclosure, the scope of which is defined only by the claims. Other examples, modifications, and usages will be apparent to those skilled in the art without departing from the spirit and scope of the claimed invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise. As used herein, the term “about” refers to a ±10% variation from the nominal value. All numerical values and ranges disclosed herein are deemed to include “about” before each value.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

1. A system, comprising:
one or more processors, the one or more processors configured to:
receive at least one first extraluminal image;
receive second extraluminal images captured during delivery of an intravascular device;
detect motion features in the at least one first extraluminal image and the second extraluminal images;
correlate, based the detected motion features, the at least one first extraluminal image and the second extraluminal images; and
provide for output real-time visualization of a position of the intravascular device on the at least one first extraluminal image or one of the second extraluminal images including the intravascular device.

2. The system of claim 1, wherein the at least one first extraluminal image is a high dose contrast x-ray angiogram.

3. The system of claim 1, wherein the second extraluminal images are low dose contrast x-ray angiograms.

4. The system of claim 1, wherein the intravascular device is at least one of a stent delivery device, a balloon device, an intravascular imaging probe, a vessel prep device, or a pressure wire.

5. The system of claim 1, wherein the one or more processors are further configured to generate, based on the at least one first extraluminal image, a vessel map.

6. The system of claim 1, wherein the one or more processors are further configured to automatically detect, using an artificial intelligence (AI) model, a working vessel.

7. The system of claim 6, wherein when training the AI model to automatically detect the working vessel, the one or more processors are further configured to:
annotate pre-contrast extraluminal images as line strips following a trajectory of at least one of a guide wire or a guide catheter;
label a path of the working vessel in post-contrast extraluminal images;
provide the annotated pre-contrast extraluminal images and labeled post-contrast extraluminal images as training data to the AI model; and
train the AI model to predict a working vessel trajectory.

8. The system of claim 6, wherein the one or more processors are further configured to:
receive, as input into the AI model, at least one pre-contrast extraluminal image;
train, by augmenting high-dose extraluminal images into low-dose extraluminal images as input, the AI model to segment at least one of a guide catheter, guide wire, stent marker, or balloon marker on the low-dose extraluminal images;
detect, by executing the AI model and based on the at least one pre-contrast extraluminal image, a guide wire of the intravascular device;
propagate, on a frame by frame basis by executing the AI model and based on the detected guide wire, wire information; and
automatically predict, by executing the AI model based on the propagated wire information, a working vessel trajectory.

9. A system, comprising,
one or more processors, the one or more processors configured to:
receive at least one first extraluminal image;
receive second extraluminal images captured during delivery of an intravascular device;
detect motion features in the at least one first extraluminal image and the second extraluminal images, wherein the motion features include at least one of a guide catheter tip, a distal endpoint of a working vessel, an optical flow at the guide catheter tip, or an optical flow at the distal endpoint of the working vessel;
correlate, based the detected motion features, the at least one first extraluminal image and the second extraluminal images; and
provide for output real-time visualization of a position of the intravascular device on the at least one first extraluminal image or one of the second extraluminal images including the intravascular device.

10. The system of claim 9, wherein when detecting the motion features the one or more processors are further configured to automatically detect, by executing a AI model, the working vessel.

11. The system of claim 9, wherein when correlating the at least one first extraluminal image and the second extraluminal images the one or more processors are further configured to:
determine vessel level motion;
determine wire tip level motion; and
determine vessel pixel level motion.

12. The system of claim 11, wherein when determining the vessel level motion the one or more processors are further configured to determine a two-dimensional translation vector.

13. The system of claim 12, wherein the two-dimensional translation vector corresponds to two-dimensional translation at an n-th frame with respect to a first frame.

14. The system of claim 12, wherein when determining the wire tip level motion the one or more processors are further configured to determine absolute spatial information and relative spatial information.

15. The system of claim 14, wherein the absolute spatial information corresponds to coordinates and the relative spatial information corresponds to optical flow between adjacent image frames.

16. The system of claim 11, wherein when determining vessel pixel level motion the one or more processors are further configured to determine absolute spatial information and relative spatial information for a plurality of points on a working vessel.

17. The system of claim 14, wherein the absolute spatial information corresponds to coordinates and the relative spatial information corresponds to optical flow between adjacent image frames.

18. A system, comprising:
one or more processors, the one or more processors configured to:
receive at least one first extraluminal image;
receive second extraluminal images captured during delivery of an intravascular device;
detect motion features in the at least one first extraluminal image and the second extraluminal images;
determine, based on the detected motion features, a heartbeat period of a patient; and
provide for output real-time visualization of a position of the intravascular device on the at least one first extraluminal image or one of the second extraluminal images including the intravascular device.

19. The system of claim 18, wherein the one or more processors are further configured to determine, based on the detected motion features, a spatial-temporal phase match between the at least one first extraluminal image and at least one of the second extraluminal images.

20. The system of claim 19, wherein the one or more processors are further configured to:
resample the detected motion features at a common frame rate;
determine a maximum correlation coefficient for pairs of motion features in the at least one first extraluminal image and the second extraluminal images; and
determine, based on the maximum correlation coefficient, a time shift.

21. The system of claim 20, wherein the one or more processors are further configured to:
determine a drift between detected motion features in the at least one first extraluminal image; and
adjust, based on the determined drift, the time shift.

22. The system of claim 20, wherein the one or more processors are further configured to:
iteratively predict the time shift; and
update, based on the iteratively predicted time shift, the time shift to a corrected time shift.

23. The system of claim 20, wherein the one or more processors are further configured to tune the spatial-temporal phase match.

24. The system of claim 23, wherein when tuning the spatial-temporal phase match the one or more processors are further configured to:
identify another first extraluminal image different than the at least one of the first extraluminal images; and
tune, based on the other first extraluminal image, the spatial-temporal phase match.

25. The system of claim 23, wherein the one or more processors are further configured to update based on the tuning, the real-time visualization to include the other first extraluminal image.

26. The system of claim 21, wherein the one or more processors are further configured to detect the intravascular device.

27. The system of claim 26, wherein when detecting the intravascular device in the second extraluminal images the one or more processors are further configured to execute a AI model.

28. The system of claim 27, wherein the one or more processors are further configured to train the AI model, wherein when training the AI model the one or more processors are further configured to:
provide as input to the AI model a co-registration dataset comprising a plurality of intraluminal images and extraluminal images, wherein the plurality of intraluminal and extraluminal images are annotated images; and
train the AI model to predict a position of the intravascular device.

29. The system of claim 28, wherein the annotated images include annotations identifying one or more intravascular device markers.

30. The system of claim 26, wherein the one or more processors are further configured to:
detect an optical flow of the intravascular device;
determine, based on the detected optical flow, a position of the intravascular device in a first frame of the second extraluminal images; and
predict, based on the detected optical flow, the position of the intravascular device in a subsequent frame of the second extraluminal images.

31. A system, comprising:
one or more processors, the one or more processors configured to:
receive at least one first extraluminal image;
receive second extraluminal images captured during delivery of an intravascular device;
detect motion features in the at least one first extraluminal image and the second extraluminal images;
correlate, based the detected motion features, the at least one first extraluminal image and the second extraluminal images;
provide for output real-time visualization of a position of the intravascular device on the at least one first extraluminal image or one of the second extraluminal images including the intravascular device; and
provide for output a treatment zone on at least one of the second extraluminal images or the at least one first extraluminal image.

32. The system of claim 31, wherein the treatment zone is at least one of a treatment device landing zone, a balloon device zone, a vessel prep device zone, or a lesion related zone.

33. The system of claim 32, wherein:
the lesion related zone is at least one of calcification frames, lipid frames, or dissected frames, and
the lesion related zone is identified from another imaging modality and co-registered, by the one or processors, to the first extraluminal image.

34. The system of claim 33, wherein the other imaging modality is an intravascular imaging modality.

35. The system of claim 31, wherein the one or more processors are further configured to receive annotations of the at least one first extraluminal image or the second extraluminal images.

36. The system of claim 35, wherein when receiving the annotations the one or more processors are further configured to:
receive one or more inputs from a user corresponding to the annotations; or
automatically determine, based on vessel data, the annotations.

37. The system of claim 35, wherein the annotations include one or more of a plaque burden, fractional flow reserve (“FFR”) measurements at one or more locations along a vessel, calcium angles, EEL detections, calcium detections, proximal frames, distal frames, EEL-based metrics, stent decisions, scores, recommendations for debulking, recommendations for subsequent procedures, stent placement zone, treatment device landing zone, balloon device zone, vessel prep device zone, or lesion related zone.

38. The system of claim 35, wherein the one or more processors are further configured to update, based on the received annotations, a second one of the at least one first extraluminal image or the second extraluminal images.

39. A system, comprising:
one or more processors, the one or more processors configured to:
receive at least one first extraluminal image;
receive second extraluminal images captured during delivery of an intravascular device;
detect motion features in the at least one first extraluminal image and the second extraluminal images;
correlate, based the detected motion features, the at least one first extraluminal image and the second extraluminal images;
provide for output real-time visualization of a position of the intravascular device on the at least one first extraluminal image or one of the second extraluminal images including the intravascular device; and
automatically capture a screen capture of the real-time visualization of the position of the intravascular device.

40. The system of claim 39, wherein the screen capture is automatically captured when the intravascular device is within a threshold distance of a region of interest.

41. The system of claim 40, wherein the region of interest is a treatment zone.

42. The system of claim 41, wherein the treatment zone is at least one of a treatment device landing zone, a balloon device zone, a vessel prep device zone, or a lesion related zone.

43. The system of claim 40, wherein determining the threshold distance comprises at least one of:
determining a number of pixels between an outer boundary of the region of interest and at least one detected marker on the intravascular device, or
determining a spatial distance between the at least one detected marker on the intravascular device and the region of interest.

44. The system of claim 43, wherein the spatial distance is a Euclidean distance or a geodesic distance.

45. The system of claim 39, wherein the one or more processors are further configured to automatically zoom a portion of the real-time visualization.

46. The system of claim 45, wherein the portion of the real-time visualization is automatically zoomed when the intravascular device is within a threshold distance of a region of interest.

47. The system of claim 46, wherein the portion of the real-time visualization corresponds to the region of interest.

48. The system of claim 47, wherein the region of interest is a treatment zone or a location of the intravascular device.

49. The system of claim 48, wherein the treatment zone is at least one of a treatment device landing zone, a balloon device zone, a vessel prep device zone, or a lesion related zone.

50. The system of claim 46, wherein determining the threshold distance comprises at least one of:
determining a number of pixels between an outer boundary of the region of interest and at least one detected marker on the intravascular device, or
determining a spatial distance between the at least one detected marker on the intravascular device and the region of interest.

51. The system of claim 50, wherein the spatial distance is a Euclidean distance or a geodesic distance.

52. The system of claim 46, wherein the automatically zooming comprises:
sorting pixel values of the real-time visualization by their intensity;
normalizing pixel intensity values lower than a predetermined threshold; and
applying a median filter to the normalized pixel intensity values of the real time visualization.

53. A system comprising:
one or more processors, the one or more processors configured to:
receive extraluminal images captured during delivery of an intravascular device, wherein the intravascular device has a radio-opaque marker;
detect a plurality of device marker candidates, wherein at least one of the device marker candidates corresponds to the radio-opaque marker of the intravascular device;
automatically detect, using an artificial intelligence (AI) model, a working vessel using a plurality of virtual boxes and at least one of the plurality of device marker candidates,
wherein at least one of the plurality of virtual boxes contains the at least one of the plurality of device marker candidates; and
select at least one of the plurality of the virtual boxes that includes a region of interest of the working vessel that contains the at least one of the plurality of device markers.

54. The system of claim 53, wherein the one or more processors are further configured to:
predict center points of each of the plurality of virtual boxes,
pair the detected device marker candidates with the nearest of the plurality of boxes;
filter the device marker candidates that are beyond boundaries of the plurality of virtual boxes;
update the center points of each of the plurality of virtual boxes using the filtered device marker candidates;
determine displacement of the predicted center points and updated center points; and
repositioning, based on the determined displacement, the plurality of virtual boxes by the determined displacement.

55. The system of claim 54, wherein the updating the center point includes approximating the center point of the virtual box using the device marker candidate.

56. The system of claim 55, wherein more than one device marker candidate is within at least one of the plurality of virtual boxes.

57. The system of claim 53, wherein the AI model is trained using annotations on high dose x-ray angiographs.

58. The system of claim 53, wherein the extraluminal images are live x-ray angiographs or fluoroscopy images.

59. The system of claim 53, wherein the one or more processors are further configured to track the region of interest during a percutaneous coronary intervention procedure.

60. The system of claim 53, wherein the one or more processors are further configured to enhance the region of interest using local contrast stretching by selectively enhancing the local contrast between the detected device marker candidate and surrounding regions.

61. The system of claim 53, wherein the one or more processors are further configured to automatically zoom the region of interest, wherein the automatic zooming comprises:
sorting pixel values of the real-time visualization by their intensity;
normalizing pixel intensity values lower than a predetermined threshold; and
applying a median filter to the normalized pixel intensity values of the real time visualization.
